(19) United States
(12) Patent Application Publication
(10) Pub. No.: US 2002/0065230 A1
(43) Pub. Date: May 30, 2002

(54) Protease inhibitors

(75) Inventors: William Edward Bondinell, Wayne, PA (US); Renee Louise Desjarlais, St. Davids, PA (US); Daniel Frank Veber, Ambler, PA (US); Dennis Shinji Yamashita, King of Prussia, PA (US)

Correspondence Address: GLAXOSMITHKLINE, Corporate Intellectual Property - UW2220, P.O. Box 1539, King of Prussia, PA, 19406-0939, US

(73) Assignee: SmithKline Beecham Corporation

(21) Appl. No.: 10/054,531

(22) Filed: Jan. 22, 2002

Related U.S. Application Data
(63) Continuation of application No. 09/423,423, filed on Nov. 8, 1999, now abandoned.
Application No. 09/423,423 is a 371 of international application No. PCT/US98/08764, filed on Apr. 30, 1998.

(51) Int. Cl. A61K 38/05; C 07D 4 3/02; C 07D 4 1/02

(52) U.S. Cl. 514/19; 546/169; 546/281.1; 548/253; 546/316

(57) ABSTRACT

The present invention provides bis-aminomethylcarbonyl compounds that are inhibitors of cysteine and serine proteases. The compounds are particularly useful for treating diseases in which excess cysteine protease activity has been implicated, including osteoporosis, periodontitis and arthritis.

### FIELD OF THE INVENTION

This invention relates in general to bis-aminomethyl carbonyl protease inhibitors, particularly such inhibitors of cysteine and serine proteases, more particularly compounds which inhibit cysteine proteases, even more particularly compounds which inhibit cysteine proteases of the papain superfamily, yet more particularly compounds which inhibit cysteine proteases of the cathepsin family, most particularly compounds which inhibit cathepsin K. Such compounds are particularly useful for treating diseases in which cysteine proteases are implicated, especially diseases of excessive bone or cartilage loss, e.g., osteoporosis, periodontitis, and arthritis.

### BACKGROUND OF THE INVENTION

Cathepsins are a family of enzymes which are part of the papain superfamily of cysteine proteases. Cathepsins B, H, L, N and S have been described in the literature. Recently, cathepsin K polypeptide and the cDNA encoding such polypeptide were disclosed in U.S. Pat. No. 5,501,969 (called cathepsin O therein). Cathepsin K has been recently expressed, purified, and characterized. Bossard, M. J., et al., (1996) J. Biol. Chem. 271, 12517-12524; Drake, F. H., et al., (1996) J. Biol. Chem. 271, 12511-12516; Bromme, D., et al., (1996) J. Biol. Chem. 271, 2126-2132.

Cathepsin K has been variously denoted as cathepsin O or cathepsin O2 in the literature. The designation cathepsin K is considered to be the more appropriate one.

Cathepsins function in the normal physiological process of protein degradation in animals, including humans, e.g., in the degradation of connective tissue. However, elevated levels of these enzymes in the body can result in pathological conditions leading to disease. Thus, cathepsins have been implicated as causative agents in various disease states, including but not limited to, infections by pneumocystis carinii, trypsanoma cruzi, trypsanoma brucei brmcei, and Crithidia fusiculata; as well as in schistosomiasis, malaria, tumor metastasis, metachromatic leukodystrophy, muscular dystrophy, amytrophy, and the like. See International Publication Number WO 94/04172, published on Mar. 3, 1994, and references cited therein. See also European Patent Application EP 0 603 873 A1, and references cited therein. Two bacterial cysteine proteases from P. gingivallis, called gingipains, have been implicated in the pathogenesis of gingivitis. Potempa, J., et al. (1994) Perspectives in Drug Discovery and Design, 2,445-458.

Cathepsin K is believed to play a causative role in diseases of excessive bone or cartilage loss. Bone is composed of a protein matrix in which spindle- or plate-shaped crystals of hydroxyapatite are incorporated. Type I collagen represents the major structural protein of bone comprising approximately 90% of the protein matrix. The remaining 10% of matrix is composed of a number of non-collagenous proteins, including osteocalcin, proteoglycans, osteopontin, osteonectin, thrombospondin, fibronectin, and bone sialoprotein. Skeletal bone undergoes remodelling at discrete foci throughout life. These foci, or remodelling units, undergo a cycle consisting of a bone resorption phase followed by a phase of bone replacement.

Bone resorption is carried out by osteoclasts, which are multinuclear cells of hematopoietic lineage. The osteoclasts adhere to the bone surface and form a tight sealing zone, followed by extensive membrane ruffling on their apical (i.e., resorbing) surface. This creates an enclosed extracellular compartment on the bone surface that is acidified by proton pumps in the ruffled membrane, and into which the osteoclast secretes proteolytic enzymes. The low pH of the compartment dissolves hydroxyapatite crystals at the bone surface, while the proteolytic enzymes digest the protein matrix. In this way, a resorption lacuna, or pit, is formed. At the end of this phase of the cycle, osteoblasts lay down a new protein matrix that is subsequently mineralized. In several disease states, such as osteoporosis and Paget's disease, the normal balance between bone resorption and formation is disrupted, and there is a net loss of bone at each cycle. Ultimately, this leads to weakening of the bone and may result in increased fracture risk with minimal trauma.

Several published studies have demonstrated that inhibitors of cysteine proteases are effective at inhibiting osteoclast-mediated bone resorption, and indicate an essential role for a cysteine proteases in bone resorption. For example, Delaisse, et al., Biochem. J., 1980, 192, 365, disclose a series of protease inhibitors in a mouse bone organ culture system and suggest that inhibitors of cysteine proteases (e.g., leupeptin, Z-Phe-Ala-CHN2) prevent bone resorption, while serine protease inhibitors were ineffective. Delaisse, et al., Biochem. Biophys. Res. Commun., 1984, 125,441, disclose that E-64 and leupeptin are also effective at preventing bone resorption in vivo, as measured by acute changes in serum calcium in rats on calcium deficient diets. Lemer, et al., J. Bone Min. Res., 1992, 7,433, disclose that cystatin, an endogenous cysteine protease inhibitor, inhibits PTH stimulated bone resorption in mouse calvariae. Other studies, such as by Delaisse, et al., Bone, 1987, 8,305, Hill, et al., J. Cell. Biochem., 1994,56, 118, and Everts, et al., J. Cell. Physiol., 1992, 150, 221, also report a correlation between inhibition of cysteine protease activity and bone resorption. Tezuka, et al., J. Biol. Chem., 1994, 269, 1106, Inaoka, et al., Biochem. Biophys. Res. Commun., 1995, 206, 89 and Shi, et al., FEBS Lett., 1995, 357, 129 disclose that under normal conditions cathepsin K, a cysteine protease, is abundantly expressed in osteoclasts and may be the major cysteine protease present in these cells.

The abundant selective expression of cathepsin K in osteoclasts strongly suggests that this enzyme is essential for bone resorption. Thus, selective inhibition of cathepsin K may provide an effective treatment for diseases of excessive bone loss, including, but not limited to, osteoporosis, gingival diseases such as gingivitis and periodontitis, Paget's disease, hypercalcemia of malignancy, and metabolic bone disease. Cathepsin K levels have also been demonstrated to be elevated in chondroclasts of osteoarhritic synovium. Thus, selective inhibition of cathepsin K may also be useful for treating diseases of excessive cartilage or matrix degradation, including, but not limited to, osteoarthritis and rheumatoid arthritis. Metastatic neoplastic cells also typically express high levels of proteolytic enzymes that degrade the surrounding matrix. Thus, selective inhibition of cathepsin K may also be useful for treating certain neoplastic diseases.

Several cysteine protease inhibitors are known. Palmer, (1995) J. Med. Chem., 38, 3193, disclose certain vinyl sulfones which irreversibly inhibit cysteine proteases, such as the cathepsins B, L, S, O2 and cruzain. Other classes of compounds, such as aldehydes, nitrites, α-ketocarbonyl compounds, halomethyl ketones, diazomethyl ketones, (acyloxy)methyl ketones, ketomethylsulfonium salts and epoxy succinyl compounds have also been reported to inhibit cysteine proteases. See Palmer, id, and references cited therein.

U.S. Pat. No. 4,518,528 discloses peptidyl fluoromethyl ketones as irreversible inhibitors of cysteine protease. Published International Patent Application No. WO 94/04172, and European Patent Application Nos. EP 0 525 420 A1, EP 0 603 873 A1, and EP 0 611 756 A2 describe alkoxymethyl and mercaptomethyl ketones which inhibit the cysteine proteases cathepsins B, H and L. International Patent Application No. PCT/US94/08868 and and European Patent Application No. EP 0 623 592 A1 describe alkoxymethyl and mercaptomethyl ketones which inhibit the cysteine protease IL-1β convertase. Alkoxymethyl and mercaptomethyl ketones have also been described as inhibitors of the serine protease kininogenase (International Patent Application No. PCT/GB91/01479).

Azapeptides which are designed to deliver the azaamino acid to the active site of serine proteases, and which possess a good leaving group, are disclosed by Elmore et al., Biochem. J., 1968, 107, 103, Garker et al., Biochem. J., 1974,139, 555, Gray et al., Tetrahedron, 1977,33, 837, Gupton et al., J. Biol. Chem., 1984,259, 4279, Powers et al., J. Biol. Chem., 1984, 259, 4288, and are known to inhibit serine proteases. In addition, J. Med. Chem., 1992, 35,4279, discloses certain azapeptide esters as cysteine protease inhibitors.

Antipain and leupeptin are described as reversible inhibitors of cysteine protease in McConnell et al., J. Med. Chem., 33, 86; and also have been disclosed as inhibitors of serine protease in Umezawa et al., 45 Meth. Enzymol. 678. E64 and its synthetic analogs are also well-known cysteine protease inhibitors (Barrett, Biochem. J., 201, 189, and Grinde, Biochem. Biophys. Acta, 701, 328).

1,3-diamnido-propanones have been described as analgesic agents in U.S. Pat. Nos. 4,749,792 and 4,638,010.

Thus, a structurally diverse variety of cysteine protease inhibitors have been identified. However, these known inhibitors are not considered suitable for use as therapeutic agents in animals, especially humans, because they suffer from various shortcomings. These shortcomings include lack of selectivity, cytotoxicity, poor solubility, and overly rapid plasma clearance. A need therefore exists for methods of treating diseases caused by pathological levels of cysteine proteases, including cathepsins, especially cathepsin K, and for novel inhibitor compounds useful in such methods.

We have now discovered a novel class of bis-aminomethyl carbonyl compounds which are protease inhibitors, most particularly of cathepsin K.

### SUMMARY OF THE INVENTION

An object of the present invention is to provide bis-aminomethyl carbonyl protease inhibitors, particularly such inhibitors of cysteine and serine proteases, more particularly such compounds which inhibit cysteine proteases, even more particularly such compounds which inhibit cysteine proteases of the papain superfamily, yet more particularly such compounds which inhibit cysteine proteases of the cathepsin family, most particularly such compounds which inhibit cathepsin K, and which are useful for treating diseases which may be therapeutically modified by altering the activity of such proteases.

Accordingly, in the first aspect, this invention provides a compound according to Formula I.

In another aspect, this invention provides a pharmaceutical composition comprising a compound according to Formula I and a pharmaceutically acceptable carrier, diluent or excipient.

In yet another aspect, this invention provides intermediates useful in the preparation of the compounds of Formula I.

In still another aspect, this invention provides a method of treating diseases in which the disease pathology may be therapeutically modified by inhibiting proteases, particularly cysteine and serine proteases, more particularly cysteine proteases, even more particularly cysteine proteases of the papain superfamily, yet more particularly cysteine proteases of the cathepsin family, most particularly cathepsin K.

In a particular aspect, the compounds of this invention are especially useful for treating diseases characterized by bone loss, such as osteoporosis and gingival diseases, such as gingivitis and periodontitis, or by excessive cartilage or matrix degradation, such as osteoarthritis and rheumatoid arthritis.

### DETAILED DESCRIPTION OF THE INVENTION

The present invention provides compounds of Formula I:

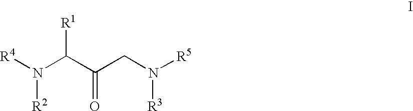

wherein:

R¹, R² and R³ are independently H; C₁₋₆ alkyl, preferably methyl or isobutyl; C₃₋₁₁cycloalkyl; C₂₋₆ alkenyl; C₂₋₆ alkynyl; Ar, preferably phenyl; Het; C₁₋₆ alkyl-Ar, preferably benzyl; C₃₋₁₁cycloalkyl-Ar; C₂₋₆ alkenyl-Ar; C₂₋₆ alkynyl-Ar; C₁₋₆ alkyl-Het, preferably isonicotinyl; C₃₋₁₁cycloalkyl-Het; C₂₋₆ alkenyl-Het; or C₂₋₆ alkynyl-Het;

R⁴ is N—(R⁶)-NHCH(C₁₋₆ alkyl)-CO, preferably N—R⁶-leucinyl-, N—R⁶-norleucinyl-, N—R⁶-norvalinyl-, N—R⁶-isoleucinyl-, N—R⁶-α-allyl-glycinyl-, N—R⁶-α-(cyclopropylmethyl)-glycinyl-, N—R⁶-β-tert-butyl-alaninyl, or N—R⁶-homo-leucinyl-; N,N—R⁶-(C₁₋₆ alkyl)-N(C₁₋₆ alkyl)-CO, preferably N,N—R⁶-methyl-leucinyl-; N—(R⁶)—NHCH(C₂₋₆ alkenyl)-CO—; N—(R⁶)—NHCH(C₂₋₆ alkynyl)-CO—; N—(R⁶)—NHCH(C₁₋₆ alkyl-Ar)—CO—; N—(R⁶)—NHCH(C₂₋₆ alkenylAr)—CO—; N—(R⁶)—NHCH(C₂₋₆ alkynyl-Ar)—CO—; N—(R⁶)—NHCH(C₁₋₆ alkyl-Het)-CO—; N—(R⁶)—NHCH(C₂₋₆ alkenyl-Het)-CO—; N—(R⁶)—NHCH(C₂₋₆ alkynyl-Het)-CO—; ArCO, preferably 3-phenoxy-benzoyl, 4-phenoxy-benzoyl-, or 2-benzyloxy benzoyl-; Ar—C₁₋₆ alkyl-CO, preferably 4-biphenyl acetyl-, 2-(4-biphenyl)-4-methyl-valeryl, 2-(3-biphenyl)-4methyl-valeryl, 1-(3-biphenyl)-but-3-ene-1-carbonyl, 1-(3-biphenyl)-ethyl-2-cyclopropane-1-carbonyl, 1-(3-biphenyl)-3-methyl-but-3-ene-1-carbonyl, 3-(2-pyridyl)-phenyl acetyl, or 3-(3-pyridyl)-phenyl acetyl; Ar-SO₂, preferably 3-phenoxy-phenyl sulfonyl, 4-phenoxy-phenyl sulfonyl, or 3-(4(3-chloro-2-cyano-phenoxy)-phenyl sulfonyl-; Ar—C₁₋₆ alkyl-SO₂; Het-CO; Het-C₁₋₆ alkyl-CO; Het-SO₂, preferably 8-quinoline sulfonyl-; or Het-C₁₋₆ alkyl-SO₂;

R⁵ is N—R⁷-amino acid, preferably N—(R⁷)-NHCH(C₁₋₆ alkyl)-CO, more preferably N—R⁷-leucinyl-, N—R⁷-norleucinyl-, N—R⁷-norvalinyl-,N—R⁷-isoleucinyl-, N—R⁷-α-allyl-glycinyl-, N—R⁷-α-(cyclopropylmethyl)-glycinyl-, N—R⁷-p-tert-butyl-alaninyl-, or N—R⁷-homo-leucinyl-, preferably N—(R⁷)-NHCH(C₂₋₆ alkenyl)-CO—, preferably N—(R⁷)—NHCH(C₂₋₆ alkynyl)-CO—, preferably N—(R⁷)—NHCH(C₁₋₆ Ar)—CO—, more preferably N—(R⁷)-phenylalaninyl-, preferably N—(R⁷)—NHCH(C₂₋₆ alkenylAr)—CO—, preferably N—(R⁷)—NHCH(C₂₋₆ alkynyl-Ar)—CO—, preferably R⁷-γ-t-butyl-glutamyl-, preferably R⁷-glutamyl-, or preferably N,N—R⁷—(C₁-C₆ alkyl)-leucinyl-; C₁₋₆ alkylCO, preferably acetyl-; C₃₋₁₁cycloalkyl-CO; ArCO, preferably benzoyl-, 3-phenoxy-benzoyl, 4-phenoxy-benzoyl-, 2-benzyloxy benzoyl-, 3-benzyloxy benzoyl-, or 4-benzyloxy benzoyl-; Ar—C₁₋₆ alkyl-CO, preferably 2-(4biphenyl)-methyl-valeryl, 2-(3-biphenyl)-4methyl-valeryl, 1-(3-biphenyl)-but-3-ene-1-carbonyl, 1-(3-biphenyl)-ethyl-2-cyclopropane-1-carbonyl, 1-(3-biphenyl)-3-methyl-but-3-ene-1-carbonyl, 1-(3-biphenyl)-but-3-ene-1-carbonyl, 3-(2-pyridyl)-phenyl acetyl, 3-(3-pyridyl)-phenyl acetyl, 4biphenyl acetyl-, or 3-biphenyl acetyl-; Ar—SO₂, preferably 3-biphenyl sulfonyl-, 4-cyano-phenyl sulfonyl, 2-carboxyl-phenyl sulfonyl, 2-carboxymethyl-phenyl sulfonyl-, 4-C-tetrazole-phenyl sulfonyl, 1-naphthalene sulfonyl, 3-phenoxy-phenyl sulfonyl, 4-phenoxy-phenyl sulfonyl, 3-(4-(3-chloro-2-cyanophenoxy)-phenyl sulfonyl-, 4-biphenyl sulfonyl-, or 2-dibenzofuran-sulfonyl; Ar—C₁₋₆ alkyl-SO₂; Het-CO, preferably 8-quinoline carbonyl-, 6-quinoline carbonyl-, 2-pyridine carbonyl, 5-(2-pyridyl)-thiophene carbonyl, N-benzyl-4-piperidinyl carbonyl, or 2-quinoline carbonyl-; Het-CH6 alkyl-CO; Het-SO₂, preferably 2-pyridyl sulfonyl, 1,3-dimethyl-5-chloro-pyrazole-4-sulfonyl, 3,5-dimethyl-isoxazole-4-sulfonyl, benzo-2,1,3-thiadiazole-4-sulfonyl, phenyl-sulfone-5-thiophene-2-sulfonyl-, 2-carboxymethyl thiophene-sulfonyl, 2,5-dichlorothiophene-3-sulfonyl-, or 8-quinoline sulfonyl; C₁₋₆ alkyl; Ar—C₀₋₆ alkyl, preferably phenyl; Het-C₀₋₆ alkyl-;

R⁶ and R⁷ are independently Ar-(C₁₋₆ alkyl)-O—CO, preferably benzyloxycarbonyl; Het-(C₁₋₆ alkyl)-O—CO, preferably 2-pyridyl methyloxycarbonyl, 3-pyridyl methyloxycarbonyl, or 4-pyridyl methyloxycarbonyl; Ar—CO, preferably benzoyl-, 1-naphthoyl-, 2-naphthoyl-, 4-phenoxy-benzoyl-, 3-phenoxy-benzoyl-, 2-phenoxy-benzoyl-, 2-chloro-benzoyl-, 4-fluoro-benzoyl, 3,4-difluoro benzoyl-, 4-trifluoromethyl benzoyl-, 2-chlorobenzoyl-, 4-carboxymethyl-benzoyl-, or 4-carboxyl-benzoyl-; Ar—SO₂; Het-CO, preferably 2-pyridyl carbonyl-, 3-pyridyl carbonyl, 4pyridyl carbonyl-, 2-quinoline carbonyl-, 3-quinoline carbonyl-, 4quinoline carbonyl-, 5-quinoline carbonyl-, 6quinoline carbonyl-, 7-quinoline carbonyl-, 8-quinoline carbonyl-, 1-isoquinoline carbonyl-, 3- isoquinoline carbonyl-, 4- isoquinoline carbonyl-, 5- isoquinoline carbonyl-, 6-isoquinoline carbonyl-, 7- isoquinoline carbonyl-, 8- isoquinoline carbonyl-, 1-benzothiophene carbonyl-, 1-benzofurancarbonyl-, 5-indole-carbonyl-sulfonyl-, N-methyl-prolinyl-, 2-quinoxaline-carbonyl-, 5-(2,3-dihydrobenzofuran-carbonyl-, 2-benzofurancarbonyl-, 2-benzothiophene-carbonyl-, N-morpholino-carbonyl-, N-methyl-piperidine-carbonyl-, or N-pyrazole-carbonyl-; Het-SO₂, preferably 2-pyridyl sulfonyl-, 3-pyridyl sulfonyl, 4-pyridyl sulfonyl, 2-quinoline sulfonyl-, 3-quinoline sulfonyl-, 4-quinoline sulfonyl-, 5-quinoline sulfonyl-, 6-quinoline sulfonyl-, 7-quinoline sulfonyl-, 8-quinoline sulfonyl-, 1- isoquinoline sulfonyl-, 3- isoquinoline sulfonyl-, 4- isoquinoline sulfonyl-, 5-isoquinoline sulfonyl-, 6- isoquinoline sulfonyl-, 7- isoquinoline sulfonyl-, or 8-isoquinoline sulfonyl-; C₁₋₆ alkyl-CO, preferably acetyl; N,N-dimethyl glycinyl-; C₃₋₁₁cycloalkyl-CO, preferably trans-4-propyl-cyclohexyl-carbonyl-, or cyclohexyl-carbonyl-; C₁₋₆ alkyl-SO₂; C₂₋₆ alkenyl-CO; C₂₋₆ alkenyl-SO₂; C₂₋₆ alkynyl-CO; C₂₋₆ alkynyl-SO₂; ArC₁₋₆ alkyl-CO; ArC₁₋₆ alkyl-SO₂; ArC₂₋₆ alkenyl-CO; ArC₂₋₆ alkenyl-SO₂; Ar—C₂₋₆ alkynyl-CO; Ar—C₂₋₆ alkynyl-SO₂; Het-C₁₋₆ alkyl-CO, preferably 4imidazole acetyl-, 2-pyridyl acetyl, 3-pyridyl acetyl, 4pyridyl acetyl-, or N-morpholine acetyl-; Het-C₁₋₆ alkyl-SO₂; Het-C₂₋₆ alkenyl-CO; Het-C₂₋₆ alkenyl-SO₂; Het-C₂₋₆ alkynyl-CO; or Het-C₂₋₆ alkynyl-SO₂; and pharmaceutically acceptable salts, hydrates and solvates thereof.

Compounds of Formula I wherein R¹, R² or R³ is H are preferred.

Even more preferred are compounds of Formula I wherein:

R¹ is H or C₁₋₆ alkyl, preferably methyl;

R² and R³ are H;

R⁴ is N—(R⁶)-NHCH(C₁₋₆ alkyl)-CO, preferably N—R⁶-ieucinyl, more preferably N-(2-pyridyl carbonyl)-leucinyl, N-(8-quinoline carbonyl)-leucinyl, N-(6-quinoline carbonyl)-leucinyl, N-(2-quinoline carbonyl)-leucinyl, N-(4-imidazole acetyl)-leucinyl, N-benzoyl-leucinyl, N-(2-pyridyl sulfonyl)-leucinyl, N-(1-isoquinoline carbonyl)-leucinyl, N-(N-morpholine acetyl)-leucinyl, N-(N-methyl prolinyl)-leucinyl, N-(N, N-dimethyl glycinyl)-leucinyl, N-(8-quinoline sulfonyl)-leucinyl, N-Cbz-leucinyl, N-pentafluorobenzoyl-leucinyl, N-2-naphthoyl-leucinyl, N-1-naphthoyl-leucinyl, N-4-fluorobenzoyl-leucinyl, N-(4-trifluoromethyl benzoyl)-leucinyl N-3,4-difluorobenzoyl-leucinyl, N-3,4-dimethoxybenzoyl-leucinyl, N-(1-benzothiophene-carbonyl)-leucinyl, N-(2-benzothiazole-carbonyl)-leucinyl, N-(5-benzothiophene-carbonyl)-leucinyl, N-(6-benzothiophene-carbonyl)-leucinyl, N-(5-indole-carbonyl)-leucinyl, N-(trans-4-propyl cyclohexyl-carbonyl)-leucinyl, N-(2-quinoxaline-carbonyl)-leucinyl, N-5-(2,3-dihydrobenzofuran)-carbonyl)-leucinyl, N-(2-benzofuran-carbonyl)-leucinyl, N-(N-methyl-2-indole-carbonyl)-leucinyl, N-(2-chloro-benzoyl-carbonyl)-leucinyl, N-(4-phenoxy-phenyl-carbonyl)-leucinyl, N-(3-methoxy-2-quinoline-carbonyl)-leucinyl, N-(2-pyridyl-methyleneoxy-carbonyl)-leucinyl or N-(cyclohexyl-carbonyl)-leucinyl; or preferably N—R⁶-norleucinyl-, more preferably N-Cbz-norleucinyl, N-(2-naphthyl-carbonyl)-norleucinyl, N-(3,4-dimethoxy-benzoyl)-norleucinyl, or N-(5-benzothiophene-carbonyl)-norleucinyl; or preferably N—R⁶-norvalinyl, more preferably N-Cbz-norvalinyl; or preferably N—R⁶-isoleucinyl, more preferably N-Cbz-isoleucinyl; or preferably N—R⁶-α-allyl-glycinyl; more preferably N-Cbz-α-allyl-glycinyl; or N,N—R⁶-(C₁₋₆ alkyl)-N(C₁₋₆ alkyl)-CO, preferably N,N—R⁶-methyl-leucinyl-, more preferably N-Cbz-N-methyl-leucinyl-; or preferably N—R⁶-α-(cyclopropylmethyl)-glycinyl-, more preferably N-Cbz-α-(cyclopropylmethyl)-glycinyl-; or preferably N—R⁶- L-β-tert-butyl-alaninyl, more preferably N-Cbz-L-p-tert-butyl-alaninyl-, or Ar—C₁₋₆ alkyl-CO, preferably 2-(3-biphenyl)-4-methyl-valeryl, or 1-(3-biphenyl)-but-3-ene-1-carbonyl, 1-(3-biphenyl)-ethyl-2-cyclopropane-1-carbonyl;

R⁵ is N—R⁷-norvalinyl-, preferably N-Cbz-norvalinyl-; Ar—C₁₋₆ alkyl-CO, preferably 3-(2-pyridyl)-phenyl acetyl, 3-(3-pyridyl)-phenyl acetyl, 3-(3-biphenyl)-3-methyl-but-3-ene-1-carbonyl, or 2-(3-biphenyl)-but-3-ene-1-carbonyl; or Het-SO₂, preferably 2-pyridyl sulfonyl, 8-quinoline sulfonyl-, 1,3-dimethyl-5-chloro-pyrazole-4-sulfonyl, 3,5-dimethyl-isoxazole 4sulfonyl, benzo-2,1,3-thiadiazole-4- sulfonyl, or 3-biphenyl sulfonyl; or Het-CO, preferably 8-quinolone carbonyl, 5-(2-pyridine)-thiophene-carbonyl, N-benzyl-4-piperidinyl carbonyl, 2-quinoline carbonyl or 2-pyridine-carbonyl; or ArCO, preferably 4-phenoxy-phenyl-carbonyl, or 2-(3-biphenyl)-3-methyl-valeryl; Ar-SO₂, preferably 2-carboxymethyl-phenyl-sulfonyl, 2-carboxyl-phenyl-sulfonyl, 4-C-tetrazole-phenyl-sulfonyl, 1-naphthalene-sulfonyl, or 2-cyano-phenyl-sulfonyl; or Ar—C₀₋₆ alkyl-, preferably phenyl.

Yet more preferred are compounds of Formula I wherein:

R¹ is H or C₁₋₆ alkyl, preferably methyl;

R² and R³ are H;

R⁴ is N—(R⁶)-NHCH(C₁₋₆ alkyl)-CO, preferably N—R⁶-leucinyl, more preferably Cbz-leucinyl, 2-naphthoyl-leucinyl, 4fluorobenzoyl-leucinyl, 3,4-dimethoxybenzoyl-leucinyl, (1-benzothiophene-carbonyl)-leucinyl, (2-quinoxaline-carbonyl)-leucinyl, 5-(2,3-dihydro-benzofuran)-carbonyl)-leucinyl, (2-benzofuran-carbonyl)-leucinyl; or N—R⁶-norleucinyl, more preferably (2-naphthyl-carbonyl)- norleucinyl, (3,4dimethoxy-benzoyl)-norleucinyl, or (5-benzothiophene-carbonyl)-norleucinyl; or Ar—C₁₋₆ alkyl-CO, preferably 2-(3-biphenyl)-4-methyl-valeryl; and

R⁵ is Ar—C₁₋₆ alkyl-CO, preferably 3-(2-pyridyl)-phenyl acetyl; or Het-SO₂, preferably 2-pyridyl sulfonyl.

Compounds of Formula I selected from the following group are particularly preferred embodiments of the present invention:

1-N-(N-(2-pyridyl carbonyl)-leucinyl)-amino-3-N-(2-pyridyl-sulfonyl)-amino-propan-2-one;

1-N-(N-(8-quinoline carbonyl)-leucinyl)-amino-3-N-(2-pyridyl-sulfonyl)-amino-propan-2-one;

1-N-(N-(2-quinoline carbonyl)-leucinyl)-amino-3-N-(2-pyridyl-sulfonyl)-amino-propan-2-one;

1-N-(N-(4-imidazole acetyl)-leucinyl)-amino-3-N-(3-biphenyl sulfonyl)-amino-propan-2-one;

1-N-(N-(2-pyridyl-carbonYl)-leucinyl)-amino-3-N-(8-quinoline carbonyl)-amino-propan-2-one;

1-N-(N-benzoyl-leucinyl)-amino-3-N-(8-quinoline carbonyl)-amnino-propan-2-one;

1-N-(N-(2-pyridyl sulfonyl)-leucinyl)-amino-3-N-(8-quinoline carbonyl)-amino-propan-2-one;

1N-(N-(8-quinoline carbonyl)-leucinyl)-amino-3-N-(8-quinoline carbonyl)-amino-propan-2-one;

1-N-(N-(1-isoquinoline-carbonyl)-leucinyl)-amino-3-N-(8quinoline carbonyl)-amnino-propan-2-one;

1-N-(N-(N-morpholine-acetyl)-leucinyl)-annino-3-N-(8-quinoline carbonyl)-amino-propan-2-one;

1-N-(N-(N-methyl prolinyl)-leucinyl)-amino-3-N-(8-quinoline carbonyl)-amino-propan-2-one;

1-N-(N-(N, N-dimethyl glycinyl)-leucinyl)-amino-3-N-(8-quinoline carbonyl)-amino-propan-2-one;

1-N-(N-(8-quinoline sulfonyl)-leucinyl)-amnino-3-N-(8-quinoline carbonyl)-amino-propan-2-one;

1-N-(N-Cbz-leucinyl)-amino-3-N-(3-(2-pyridyl)-phenyl acetyl)-amino-propan-2-one;

1-N-(N-pentafluorobenzoyl-leucinyl)-amino-3-N-(3-(2-pyridyl)-phenyl acetyl)-amino-propan-2-one;

1-N-(N-2-naphthoyl-leucinyl)-amino-3-N-(3-(2-pyridyl)-phenyl acetyl)-amino-propan-2-one;

1-N-(N-1-naphthoyl-leucinyl)-amino-3-N-(3-(2-pyridyl)-phenyl acetyl)-amino-propan-2-one;

1-N-(N-(2-pyridyl-carbonyl)-leucinyl)-amino-3-N-(3-(2-pyridyl)-phenyl acetyl)-amino-propan-2-one;

1-N-(N-4fluorobenzoyl-leucinyl)-amino-3-N-(3-(2-pyridyl)-phenyl acetyl)-amino-propan-2-one;

1-N-(N-3,4difluorobenzoyl-leucinyl)-amino-3-N-(3-(2-pyridyl)-phenyl acetyl)-amino-propan-2-one;

1-N-(N-3,4-dimethoxybenzoyl-leucinyl)-amino-3-N-(3-(2-pyridyl)-phenyl acetyl)-amino-propan-2-one;

1-N-(N-(1-benzothiophene-carbonyl)-leucinyl)-amino-3-N-(3-(2-pyridyl)-phenyl acetyl)-amino-propan-2-one;

1-N-(N-(5-indole-carbonyl)-leucinyl)-amino-3-N-(3-(2-pyridyl)-phenyl acetyl)-amino-propan-2-one;

1-N-(N-Cbz-isoleucinyl)-amino-3-N-(3-(2-pyridyl)-phenyl acetyl)-amino-propan-2-one;

1-N-(N-Cbz-norvalinyl)-amino-3-N-(3-(2-pyridyl)-phenyl acetyl)-amino-propan-2-one;

1-N-(N-Cbz-α-allyl-glycinyl)-amino-3-N-(3-(2-pyridyl)-phenyl acetyl)-amino-propan-2-one;

1-N-(N-Cbz-norleucinyl)-amino-3-N-(3-(2-pyridyl)-phenyl acetyl)-amiino-propan-2-one;

1-N-(N-Cbz-N-methyl-leucinyl)-amino-3-N-(3-(2-pyridyl)-phenyl acetyl)-amino-propan-2-one;

1-N-(N-Cbz-α-(cyclopropyl)-methyl-glycinyl)-amino-3-N-(3-(2-pyridyl)-phenyl acetyl)-amino-propan-2-one;

1-N-(N-benzyloxycarbonyl-L-O-tert-butylalanine)-amino-3-N-(3-(2-pyridyl)-phenyl acetyl)-amino-propan-2-one;

1-N-(2-(3-biphenyl) methyl-valeryl)-amino-3-N-(2-pyridyi-sulfonyl)-amino-propan-2-one;

1-N-(2-(3-biphenyl)-4-methyl-valeryl)-arino-3-N-(2-carboxymethyl-phenyl-sulfonyl)-amino-propan-2-one;

1-N-(2-(3-biphenyl)-4-methyl-valeryl)-amino-3-N-(4-cyano-phenyl-sulfonyl)-amino-propan-2-one;

1-N-(2-(3-biphenyl)-4-methyl-valeryl)-amino-3-N-(8-quinoline carbonyl)-aminopropan-2-one;

1-N-(2-(3-biphenyl)-4-methyl-valeryl)-amino-3-N-(3-(2-pyridyl)-phenyl acetyl)-amino-propan-2-one;

1-N-(2-(3-biphenyl)-4methyl-valeryl)-amino-3-N-(3-(3-pyridyl)-3-phenyl acetyl)-amino-propan-2-one;

1-N-(2-(3-biphenyl)-4-methyl-valeryl)-amino-3-N-(2-pyridine carbonyl)-amino-propan-2-one;

1-N-(2-(3-biphenyl)-4-methyl-valeryl)-amino-3-N-(5-(2-pyridine)-thiophene-carbonyl)-amino-propan-2-one;

1-N-(2-(3-biphenyl)-4-methyl-valeryl)-amino-3-N-(N-benzyl-4-piperidine-carbonyl)-amino-propan-2-one;

1-N-(2-(3-biphenyl)-4-methyl-valeryl)-amino-3-N-(2-quinoline-carbonyl)-amino-propan-2-one;

1-N-(2-(3-biphenyl)-4-methyl-valeryl)-amino-3-N-(2-carboxyl-phenyi-sulfonyl)-amino-propan-2-one;

1-N-(2-(3-biphenyl)-4-methyl-valeryl)-amino-3-N-(4-C-tetrazole-phenyl-sulfonyl)-amino-propan-2-one;

1-N-(2-(3-biphenyl)-4-methyl-valeryl)-amino-3-N-(3-(2-pyridyl-(phenyl acetyl)-amino-(S)-butan-2-one;

1-N-(2-(3-biphenyl)-3-methyl-valeryl)-1-N-methyl-amino-3-N-(3-(2-pyridyl-(phenyl acetyl)-amino-propan-2-one;

1-N-(N-2-pyridyl carbonyl-leucinyl)-amino-3-N-(4-phenoxy-phenyl carbonyl)-amino-propan-2-one;

1-N-(N-8-quinoline-carbonyl-leucinyl)-amino-3-N-(4-phenoxy-phenyl carbonyl)-amino-propan-2-one;

1-N-(N-2-quinoline-carbonyl-leucinyl)-amino-3-N-(4-phenoxy-phenyl carbonyl)-amino propan-2-one;

1-N-(N-(Cbz-norvalinyl)-amino-3-N-(8-quinoline-sulfonyl)-amino-propan-2-one;

1-N-(8-quinoline-sulfonyl)-amino-3-N-(8-quinoline-sulfonyl)-amino-propan-2-one;

1-N-(2-(3-biphenyl)-3-methyl-valeryl)-amino-3-N-(8-quinoline-sulfonyl)-amino-propan-2-one;

1-N-(2-(3-biphenyl)-3-methyl-valeryl)-amino-3-N-(2-(3-biphenyl)-3-methyl-valeryl)-amino-propan-2-one;

1-N-(N-(Cbz-norvalinyl)-amino-3-N-(N-(Cbz-norvalinyl)-amino-propan-2-one;

1-N-(1-(3-biphenyl)-but-3-ene-1-carbonyl)-amino-3-N-(3-(2-pyridyl)-phenyl acetyl)-amino-propan-2-one;

1-N-(1-(3-biphenyl)-but-3-ene-1-carbonyl)-amino-3-N-(1-(3-biphenyl)-but-3-ene-1-carbonyl)-propan-2-one;

1-N-(1-(3-biphenyl)-ethyl-2-cyclopropane-1-carbonyl)-amino-3-N-(3-(2-pyridyl)-phenyl acetyl)-amino-propan-2-one;

1-N-(2-(3-biphenyl)-3-methyl-but-3-ene-1-carbonyl)-amino-3-N-(3-(2-pyridyl)-phenyl acetyl)-amino-propan-2-one;

1-N-(1-(3-biphenyl)-3-methyl-but-3-ene-1-carbonyl)-amino-3-N-(1-(3-biphenyl)-3-methyl-but-3-ene-1-carbonyl)-amino-propan-2-one;

1-N-(Ntranspropyl cyclohexyl-carbonyl)-leucinyl)-amino-3-N-(3-(2-pyridyl)-phenyl acetyl)-amino-propan-2-one;

1-N-(N-(2-quinoxaline-carbonyl)-leucinyl)-amino-3-N-(3-(2-pyridyl)-phenyl acetyl)-amino-propan-2-one;

1-N-(N-(5-(2,3-dihydro-benzofuran)-carbonyl)-leucinyl)-amino-3-N-(3-(2-pyridyl)-phenyl acetyl)-amino-propan-2-one;

1-N-(N-(N-methyl-2-indole-carbonyl)-leucinyl)-amino-3-N-(3-(2-pyridyl)-phenyl acetyl)-amino-propan-2-one;

1-N-(N-(cyclohexyl-carbonyl)-leucinyl)-amino-3-N-(3-(2-pyridyl)-phenyl acetyl)-amino-propan-2-one;

1-N-(N-(2-chloro-benzoyl)-leucinyl)-amino-3-N-(3-(2-pyridyl)-phenyl acetyl)-amino-propan-2-one;

1-N-(N-(2-benzofuran-carbonyl)-leucinyl)-amino-3-N-(3-(2-pyridyl)-phenyl acetyl)-amino-propan-2-one;

1-N-(N-(3-phenoxy-phenyl-carbonyl)-leucinyl)-amino-3-N-(3-(2-pyridyl)-phenyl acetyl)-amino-propan-2-one;

1-N-(N-(4-phenoxy-phenyl-carbonyl)-leucinyl)-amino-3-N-(3-(2-pyridyl)-phenyl acetyl)-amino-propan-2-one;

1-N-(N-(3-methoxy-2-quinoline-carbonyl)-leucinyl)-amino-3-N-(3-(2-pyridyl)-phenyl acetyl)-amino-propan-2-one;

1-N-(N-Cbz-leucinyl)-amino-3-N-(3-(2-pyridyl-(phenyl acetyl)-amino-(S)-butan-2-one;

1-N-(N-(4fluorobenzoyl)-leucinyl)-amino-3-N-(3-(2-pyridyl-(phenyl acetyl)-amino-(S)-butan-2-one;

1-N-(N-(2-benzotbiophene-carbonyl)-leucinyl)-amino-3-N-(3-(2-pyridyl-(phenyl acetyl)-amino-(S)-butan-2-one;

1-N-(N-(2-pyridyl-methyleneoxy-carbonyl)-leucinyl)-amino-3-N-(1-naphthalene sulfonyl)-amino-propan-2-one;

1-N-(N-(2-pyridyl-methyleneoxy-carbonyl)-leucinyl)-amino-3-N-(1,3-dimethyl-S-chloro-pyrazole-4-sulfonyl)-amino-propan-2-one;

1-N-(N-(2-pyridyl-methyleneoxy-carbonyl)-leucinyl)-amino-3-N-(benzo-2, 1,3-thiadiazole-4-sulfonyl)-amino-propan-2-one;

1-N-(N-(2-pyridyl-methyleneoxy-carbonyl)-leucinyl)-aniino-3-N-(3,5-dimethyl-isoxazole-4-sulfonyl)-amino-propan-2-one;

1-N-(N-(4-trifluoromethyl benzoyl)-leucinyl)-amino-3-N-(3-(2-pyridyl)-phenyl acetyl)-amino-propan-2-one;

1-N-(N-(6-benzthiazole-carbonyl)-leucinyl)-amino-3-N-(3-(2-pyridyl)-phenyl acetyl)-amino-propan-2-one;

1-N-(N-(6-quinoline-carbonyl)-leucinyl)-amino-3-N-(3-(2-pyridyl)-phenyl acetyl)-amino-propan-2-one;

1-N-(N-(4-fluoro-benzoyl)-norleucinyl)-amino-3-N-(3-(2-pyridyl)-phenyl acetyl)-amino-propan-2-one;

1-N-(N-(2-naphthyl-carbonyl)-norieucinyl)-amino-3-N-(3-(2-pyridyl)-phenyl acetyl)-amino-propan-2-one;

1-N-(N-(3,4dimethoxy-benzoyl)-norleucinyl)-amino-3-N-(3-(2-pyridyl)-phenyl acetyl)-amino-propan-2-one;

1-N-(N-(5-benzothiophene-carbonyl)-norleucinyl)-amino-3-N-(3-(2-pyridyl)-phenyl acetyl)-amino-propan-2-one; and

(S)-3-N-(N-Cbz-leucinyl)-amino-1-N-(phenyl)-5-methyl-hexan-2-one.

Compounds of Formula I selected from the following group are most preferred embodiments of the present invention:

1-N-(N-Cbz-leucinyl)-amino-3-N-(3-(2-pyridyl)-phenyl acetyl)-amino-propan-2-one;

1-N-(N-2-naphthoy-leucinyl)-amino-3-N-(3-(2-pyridyl)-phenyl acetyl)-amino-propan-2-one;

1-N-(N-4-fluorobenzoyl-leucinyl)-amino-3-N-(3-(2-pyridyl)-phenyl acetyl)-amino-propan-2-one;

1-N-(N-3,4-dimethoxybenzoyl-leucinyl)-amino-3-N-(3-(2-pyridyl)-phenyl acetyl)-amino-propan-2-one;

1-N-(N-(1-benzothiophene-carbonyl)-leucinyl)-amino-3-N-(3-(2-pyridyl)-phenyl acetyl)-amino-propan-2-one;

1-N-(N-(5-indole-carbonyl)-leucinyl)-amino-3-N-(3-(2-pyridyl)-phenyl acetyl)-amino-propan-2-one;

1-N-(2-(3-biphenyl)-4-methyl-valeryl)-amino-3-N-(2-pyridyl-sulfonyl)-amino-propan-2-one;

1-N-(2-(3-biphenyl)-4methyl-valeryl)-amino-3-N-(3-(2-pyridyl)-phenyl acetyl)-amino-propan-2-one;

1-N-(N-(2-quinoxaline-carbonyl)-leucinyl)-amino-3-N-(3-(2-pyridyl)-phenyl acetyl)-amino-propan-2-one;

1-N-(N-(5-(2,3-dihydro-benzofuran)-carbonyl)-leucinyl)-amino-3-N-(3-(2-pyridyl)-phenyl acetyl)-amino-propan-2-one;

1-N-(N-(2-benzofuran-carbonyl)-leucinyl)-amino-3-N-(3-(2-pyridyl)-phenyl acetyl)-amino-propan-2-one;

1-N-(N-Cbz-leucinyl)-amino-3-N-(3-(2-pyridyl-(phenyl acetyl)-amino-(S)-butan-2-one;

1-N-(N-(2-benzothiophene-carbonyl)-leucinyl)-amino-3-N-(3-(2-pyridyl-(phenyl acetyl)-amino-(S) butan-2-one;

1-N-(N-(4-trifluoromethyl benzoyl)-leucinyl)-amino-3-N-(3-(2-pyridyl)-phenyl acetyl)-amino-propan-2-one;

1-N-(N-(2-naphthyl-carbonyl)-norleucinyl)-amino-3-N-(3-(2-pyridyl)-phenyl acetyl)-amino-propan-2-one;

1-N-(N-(3,4dimethoxy-benzoyl)-norleucinyl)-amino-3-N-(3-(2-pyridyl)-phenyl acetyl)-amino-propan-2-one; and

1-N-(N-(5-benzothiophene-carbonyl)-norleucinyl)-amino-3-N-(3-(2-pyridyl)-phenyl acetyl)-amino-propan-2-one.

### Definitions

The present invention includes all hydrates, solvates, complexes and prodrugs of the compounds of this invention. Prodrugs are any covalently bonded compounds which release the active parent drug according to Formula I in vivo. If a chiral center or another form of an isomeric center is present in a compound of the present invention, all forms of such isomer or isomers, including enantiomers and diastereomers, are intended to be covered herein. Inventive compounds containing a chiral center may be used as a racemic mixture, an enantiomerically enriched mixture, or the racemic mixture may be separated using well-known techniques and an individual enantiomer may be used alone. In cases in which compounds have unsaturated carbon-carbon double bonds, both the cis (Z) and trans (E) isomers are within the scope of this invention. In cases wherein compounds may exist in tautomeric forms, such as keto-enol tautomers, each tautomeric form is contemplated as being included within this invention whether existing in equilibrium or predominantly in one form.

The meaning of any substituent at any one occurrence in Formula I or any subformula thereof is independent of its meaning, or any other substituent's meaning, at any other occurrence, unless specified otherwise.

Abbreviations and symbols commonly used in the peptide and chemical arts are used herein to describe the compounds of the present invention. In general, the amino acid abbreviations follow the IUPAC-IUB Joint Commission on Biochemical Nomenclature as described in Eur. J. Biochem., 158, 9 (1984).

The term “amino acid” as used herein refers to the D- or L-isomers of alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine and valine.

“C₁₋₆alkyl” as applied herein is meant to include substituted and unsubstituted methyl, ethyl, n-propyl, isopropyl, n-butyl, isobutyl and t-butyl, pentyl, n-pentyl, isopentyl, neopentyl and hexyl and the simple aliphatic isomers thereof. Any C₁₋₆alkyl group may be optionally substituted independently by one to five halogens, SR′, OR′, N(R′)₂, C(O)N(R′)₂, carbamyl or C₁₋₄alkyl, where R′ is C₁₋₆alkyl. C₀alkyl means that no alkyl group is present in the moiety. Thus, Ar—C₀alkyl is equivalent to Ar.

“C₃₋₁₁cycloalkyl” as applied herein is meant to include substituted and unsubstituted cyclopropane, cyclobutane, cyclopentane, cyclohexane, cycloheptane, cyclooctane, cyclononane, cyclodecane, cycloundecane.

“C₂₋₆ alkenyl” as applied herein means an alkyl group of 2 to 6 carbons wherein a carbon-carbon single bond is replaced by a carbon-carbon double bond. C₂₋₆alkenyl includes ethylene, 1-propene, 2-propene, 1-butene, 2-butene, isobutene and the several isomeric pentenes and hexenes. Both cis and trans isomers are included.

“C₂₋₆alkynyl” means an alkyl group of 2 to 6 carbons wherein one carbon-carbon single bond is replaced by a carbon-carbon triple bond. C₂₋₆ alkynyl includes acetylene, 1-propyne, 2-propyne, 1-butyne, 2-butyne, 3-butyne and the simple isomers of pentyne and hexyne.

“Halogen” means F, Cl, Br, and I.

“Ar” or “aryl” means phenyl or naphthyl, optionally substituted by one or more of Ph-C₀₋₆alkyl; Het-C₀₋₆alkyl; C₁₋₆alkoxy; Ph-C₀₋₆alkoxy; Het-C₀₋₆alkoxy; OH, (CH₂)₁₋₆NR⁸R⁹; O(CH₂)₁₋₆NR⁸R⁹; C₁₋₆alkyl, OR′, N(R′)₂, SR′, CF₃, NO₂, CN, CO₂R′, CON(R′), F, Cl, Br or I; where R⁸ and R⁹ are H, C₁₋₆alkyl, Ph-C₀₋₆alkyl, naphthyl-C₀₋₆alkyl or Het-C₀₋₆alkyl; and R′ is phenyl, naphthyl or C₁₋₆alkyl.

As used herein “Het” or “heterocyclic” represents a stable 5- to 7-membered monocyclic, a stable 7- to 10-membered bicyclic, or a stable 11- to 18-membered tricyclic heterocyclic ring which is either saturated or unsaturated, and which consists of carbon atoms and from one to three heteroatoms selected from the group consisting of N, O and S, and wherein the nitrogen and sulfur heteroatoms may optionally be oxidized, and the nitrogen heteroatom may optionally be quaternized, and including any bicyclic group in which any of the above-defined heterocyclic rings is fused to a benzene ring. The heterocyclic ring may be attached at any heteroatom or carbon atom which results in the creation of a stable structure, and may optionally be substituted with one or two moieties selected from C₀₋₆Ar, C₁₋₆alkyl, OR′, N(R′)₂, SR′, CF₃, NO₂, CN, CO₂R′, CON(R′), F, Cl, Br and I, where R′ is phenyl, naphthyl, or C₁₋₆alkyl. Examples of such heterocycles include piperidinyl, piperazinyl, 2-oxopiperazinyl, 2-oxopiperidinyl, 2-oxopyrrolodinyl, 2-oxoazepinyl, azepinyl, pyrrolyl, 4-piperidonyl, pyrrolidinyl, pyrazolyl, pyrazolidinyl, imidazolyl, pyridyl, pyrazinyl, oxazolidinyl, oxazolinyl, oxazolyl, isoxazolyl, morpholinyl, thiazolidinyl, thiazolinyl, thiazolyl, quinuclidinyl, indolyl, quinolinyl, isoquinolinyl, benzimidazolyl, benzopyranyl, benzoxazolyl, furyl, pyranyl, tetrahydrofuryl, tetrahydropyranyl, thienyl, benzoxazolyl, thiamorpholinyl sulfoxide, thiamorpholinyl sulfone, and oxadiazolyl.

“HetAr” or “heteroaryl” means any heterocyclic moiety encompassed by the above definition of Het which is aromatic in character, e.g., pyridine.

It will be appreciated that the heterocyclic ring described when N═

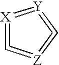

includes thiazoles, oxazoles, triazoles, thiadiazoles, oxadiazoles, isoxazoles, isothiazols, imidazoles, pyrazines, pyridazines, pyrimidines, triazines and tetrazines which are available by routine chemical synthesis and are stable. The single and double bonds (i.e., [symbol] ) in such heterocycles are arranged based upon the heteroatoms present so that the heterocycle is aromatic (e.g., it is a heteroaryl group). The term heteroatom as applied herein refers to oxygen, nitrogen and sulfur.

Here and throughout this application the term C₀ denotes the absence of the substituent group immediately following; for instance, in the moiety ArC₀₋₆alkyl, when C is 0, the substituent is Ar, e.g., phenyl. Conversely, when the moiety ArC₀₋₆alkyl is identified as a specific aromatic group, e.g., phenyl, it is understood that C is 0.

Certain radical groups are abbreviated herein. t-Bu refers to the tertiary butyl radical, Boc refers to the t-butyloxycarbonyl radical, Fmoc refers to the fluorenylmethoxycarbonyl radical, Ph refers to the phenyl radical, Cbz refers to the benzyloxycarbonyl radical.

Certain reagents are abbreviated herein. DCC refers to dicyclohexylcarbodiimide, DMAP is 2,6-dimethylaminopyridine, EDC refers to N-ethyl-N′(dimethylarinopropyl)-carbodiimide. HOBT refers to 1-hydroxybenzotriazole, DMF refers to dimethyl formamide, BOP refers to benzotriazol-1-yloxy-tris(dimethylamino)phosphonium hexafluorophosphate, DMAP is dimethylaminopyridine, NMM is N-methylmorpholine, TFA refers to trifluoroacetic acid, THF refers to tetrahydrofuran. Jones reagent is a solution of chromium trioxide, water, and sulfuric acid well-known in the art.

### Methods of Preparation

The compounds of the present invention may be conveniently prepared by the methods set forth in Schemes 1-5 below.

[chemical structure]

1,3-Diamino-propan-2-ol (or an N-alkyl substituted diamino-propanol) 1-Scheme 1 is coupled to a protected amino acid (either Cbz- or Boc-) 2-Scheme 1 to provide an intermediate amine 3-Scheme 1. Another carboxylic acid or a sulfonyl chloride is then coupled to form alcohol 4Scheme 1. (Or the two couplings are done in a single reaction pot.) Removal of the protective group provides amine 5-Scheme 1. Acylation or sulfonylation gives alcohol 6-Scheme 1, and oxidation of the alcohol provides the desired compounds 7-Scheme 1.

[chemical structure]

1,3-Diamino-propan-2-ol (or an N-alkyl substituted diamino-propanol) 1-Scheme 2 is coupled to a protected Cbz-amino acid 2-Scheme 2 to form intermediate amine 3-Scheme 2. Another carboxylic acid or sulfonyl chloride is then coupled to provide alcohol 4Scheme 2. (Or the two couplings are carried out in a single reaction pot.) Oxidation of the alcohol provides the desired compounds 5-Scheme 2.

[chemical structure]

1,3-Diamino-propan-2-ol (or an N-alkyl substituted diamino-propanol) 1-Scheme 3 is coupled to a protected either a single carboxylic acid (R═R′), 2 different carboxylic acids, a carboxylic acid and a sulfonyl chloride, a single sulfonyl chloride, or 2 different sulfonyl chlorides, followed by oxidation of the alcohols to the ketones to provide the desired compounds 2-Scheme 3,3-Scheme 3, and 4-Scheme 3, which are then purifed by silica gel chromatography.

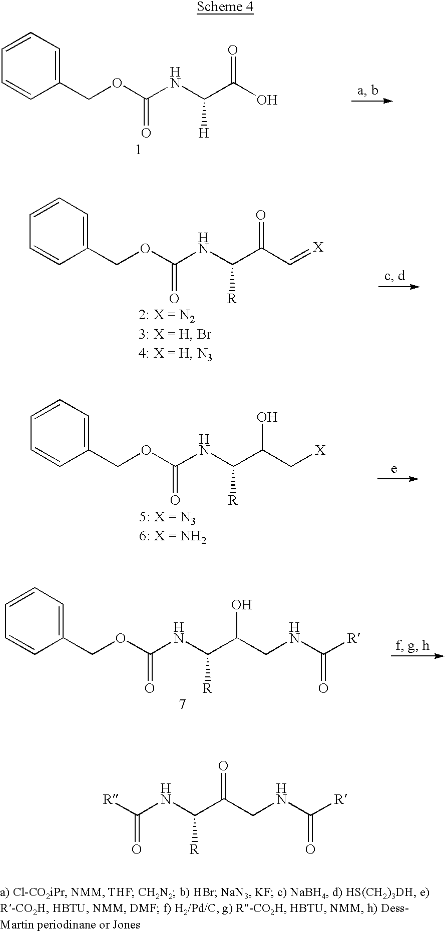

Propan-2-ones substituted at the alpha position with, for instance alkyl groups, can be prepared by converting an N-protected amino acid 1-Scheme 4, to its bromo methyl ketone 3-Scheme 4 via a diazo methyl ketone 2-Scheme 4. Then, the bromide 3-Scheme 4 is displaced with sodium azide to give the corresponding azide 4-Scheme 4. Reduction of the carbonyl with a reducing agent such as sodium borohydride gives an azido alcohol 5-Scheme 4, which is further reduced of the azide with a reducing agent such as 1,3-propandithiol gives the free amine 6-Scheme 4. Acylation or sulfonylation of the amine gives amide or sulfonamide 7-Scheme 4. Finally, deprotection, acylation, and oxidation of the carbinol with an oxidant such as Dess-Martin periodinane or Jones gives the desired compounds.

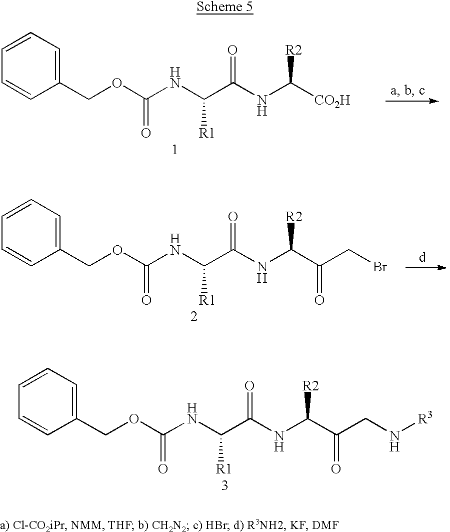

Propan-2-ones substituted at the alpha position with an N-aryl or alkyl group can be prepared by converting an N-protected di-amino acid 1-Scheme 5, to its bromo methyl ketone 2-Scheme 5 via a diazo methyl ketone. Then, the bromide 2-Scheme 5 is displaced with an amine such as aniline with potassium fluoride (or silver salt such as Ag₂O) to give the corresponding amine 3-Scheme 5.

Dess-Martin periodinane oxidation is described in J. Org. Chem. 1983, 48, 4155-4156.

Referring to the methods of preparing the compounds of Formula I set forth in Schemes 1-5 above, the skilled artisan will appreciate that the present invention includes all novel intermediates required to make the compounds of Formula I. Specifically, the present invention includes all diamino-propan-2-ols of Formula II, corresponding to the compounds of Formula I.

More specifically, the present invention provides compounds of Formula II:

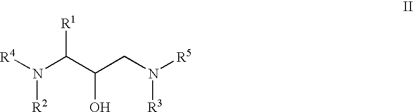

wherein:

R¹, R² and R³ are independently H; C₁₋₆ alkyl, preferably methyl or isobutyl; C₃₋₁₁cycloalkyl; C₂₋₆ alkenyl; C₂₋₆ alkynyl; Ar, preferably phenyl; Het; C₁₋₆ alkyl-Ar, preferably benzyl; C₃₋₁₁ cycloalkyl-Ar; C₂₋₆ alkenyl-Ar; C₂₋₆ alkynyl-Ar; C₁₋₆ alkyl-Het, preferably isonicotinyl; C₃₋₁₁cycloalkyl-Het; C₂₋₆ alkenyl-Het; or C₂₋₆ alkynyl-Het;

R⁴ is N—(R⁶)—NHCH(C₁₋₆ alkyl)-CO, preferably N—R⁶-leucinyl-, N—R⁶-norleucinyl-, N—R⁶-norvalinyl-, N—R⁶-isoleucinyl-, N—R⁶-cx-allyl-glycinyl-, N—R⁶-α-(cyclopropylmethyl)-glycinyl-, N—R⁶-β-tert-butyl-alaninyl, or N—R⁶-homo-leucinyl-; N,N—R⁶-(C₁₋₆ alkyl)-N(C₁₋₆ alkyl)-CO, preferably N,N—R⁶-methyl-leucinyl-; N—(R⁶)—NHCH(C₂₋₆ alkenyl)-CO—; N—(R⁶)—NHCH(C₂₋₆ alkynyl)-CO—; N—(R⁶)—NHCH(C₁₋₆ alkyl-Ar)—CO—; N—(R⁶)—NHCH(C₂₋₆ alkenylAr)—CO—; N—(R⁶)—NHCH(C₂₋₆ alkynyl-Ar)—CO—; N—(R⁶)—NHCH(C₁₋₆ alkyl-Het)-CO—; N—(R⁶)—NHCH(C₂₋₆ alkenyl-Het)-CO—; N—(R⁶)—NHCH(C₂₋₆ alkynyl-Het)-CO—; ArCO, preferably 3-phenoxy-benzoyl, 4-phenoxy-benzoyl-, or 2-benzyloxy benzoyl-; Ar—C₁₋₆ alkyl-CO, preferably 4-biphenyl acetyl-, 2-(4-biphenyl)-4-methyl-valeryl, 2-(3-biphenyl)-4methyl-valeryl, 1-(3-biphenyl)-but-3-ene-1-carbonyl, 1-(3-biphenyl)-ethyl-2-cyclopropane-1-carbonyl, 1-(3-biphenyl)-3-methyl-but-3-ene-1-carbonyl, 3-(2-pyridyl)-phenyl acetyl, or 3-(3-pyridyl)-phenyl acetyl; Ar—SO₂, preferably 3-phenoxy-phenyl sulfonyl, 4-phenoxy-phenyl sulfonyl, or 3-(4-(3-chloro-2-cyano-phenoxy)-phenyl sulfonyl-; Ar—C₁₋₆ alkyl-SO₂; Het-CO; Het-C₁₋₆ alkyl-CO; Het-SO₂, preferably 8-quinoline sulfonyl-; or Het-C₁₋₆ alkyl-SO₂;

R⁵ is N—R⁷-amino acid, preferably N—(R⁷)—NHCH(C₁₋₆ alkyl)-CO, more preferably N—R⁷-leucinyl-, N—R⁷-norleucinyl-, N—R⁷-norvalinyl-,N—R⁷-isoleucinyl-, N—R⁷-α-allyl-glycinyl-, N—R⁷-α-(cyclopropylmethyl)-glycinyl-, N—R⁷-p-tert-butyl-alaninyl-, or N—R⁷-homo-leucinyl-, preferably N—(R⁷)—NHCH(C₂₋₆ alkenyl)-CO—, preferably N—(R⁷)—NHCH(C₂₋₆ alkynyl)-CO—, preferably N—(R⁷)—NHCH(C₁₋₆ alkyl-Ar)—CO—, more preferably N—(R⁷)-phenylalaninyl-, preferably N—(R⁷)—NHCH(C₂₋₆ alkenylAr)—CO—, preferably N—(R⁷)—NHCH(C₂₋₆ alkynyl-Ar)—CO—, preferably R⁷-γ-butyl-glutamyl-, preferably R⁷-glutamyl-, or preferably N,N—R⁷—(C₁-C₆ alkyl)-leucinyl-; C₁₋₆ alkylCO, preferably acetyl-; C₃₋₁₁cycloalkyl-CO; ArCO, preferably benzoyl-, 3-phenoxy-benzoyl, 4-phenoxy-benzoyl-, 2-benzyloxy benzoyl-, 3-benzyloxy benzoyl-, or 4-benzyloxy benzoyl-; Ar—C₁₋₆ alkyl-CO, preferably 2-(4-biphenyl)-4-methyl-valeryl, 2-(3-biphenyl)-4-methyl-valeryl, 1-(3-biphenyl)-but-3-ene-1-carbonyl, 1-(3-biphenyl)-ethyl-2-cyclopropane-1-carbonyl, 1-(3-biphenyl)-3-methyl-but-3-ene-1-carbonyl, 1-(3-biphenyl)-but-3-ene-1-carbonyl, 3-(2-pyridyl)-phenyl acetyl, 3-(3-pyridyl)-phenyl acetyl, 4-biphenyl acetyl-, or 3-biphenyl acetyl-Ar—SO₂, preferably 3-biphenyl sulfonyl-, 4-cyano-phenyl sulfonyl, 2-carboxyl-phenyl sulfonyl, 2-carboxymethyl-phenyl sulfonyl-, 4-C-tetrazole-phenyl sulfonyl, 1-naphthalene sulfonyl, 3-phenoxy-phenyl sulfonyl, 4phenoxy-phenyl sulfonyl, 3-(4-(3-chloro-2-cyanophenoxy)-phenyl sulfonyl-, 4biphenyl sulfonyl-, or 2-dibenzofuran-sulfonyl; Ar—C₁₋₆ alkyl-SO₂; Het-CO, preferably 8-quinoline carbonyl-, 6-quinoline carbonyl-, 2-pyridine carbonyl, 5-(2-pyridyl)-thiophene carbonyl, N-benzyl-4-piperidinyl carbonyl, or 2-quinoline carbonyl-; Het-C₁₋₆ alkyl-CO; Het-SO₂, preferably 2-pyridyl sulfonyl, 1,3-dimethyl-5-chloro-pyrazole-4-sulfonyl, 3,5-dimethyl-isoxazole-4-sulfonyl, benzo-2,1,3-thiadiazole-4-sulfonyl, phenyl-sulfone-5-thiophene-2-sulfonyl-, 2-carboxymethyl thiophene-sulfonyl, 2,5-dichlorothiophene-3-sulfonyl-, or 8-quinoline sulfonyl; C₁₋₆ alkyl; Ar—C₀₋₆ alkyl, preferably phenyl; Het-C₀₋₆ alkyl-;

R⁶ and R⁷ are independently Ar—(C₁₋₆ alkyl)-O—CO, preferably benzyloxycarbonyl; Het-(C₁₋₆ alkyl)-O—CO, preferably 2-pyridyl methyloxycarbonyl, 3-pyridyl methyloxycarbonyl, or 4pyridyl methyloxycarbonyl; Ar—CO, preferably benzoyl-, 1-naphthoyl-, 2-naphthoyl-, 4-phenoxy-benzoyl-, 3-phenoxy-benzoyl-, 2-phenoxy-benzoyl-, 2-chloro-benzoyl-, 4-fluoro-benzoyl, 3,4-difluoro benzoyl-, 4-trifluoromethyl benzoyl-, 2-chlorobenzoyl-, 4-carboxymethyl-benzoyl-, or 4-carboxyl-benzoyl-; Ar—SO₂; Het-CO, preferably 2-pyridyl carbonyl-, 3-pyridyl carbonyl, 4-pyridyl carbonyl-, 2-quinoline carbonyl-, 3-quinoline carbonyl-, 4-quinoline carbonyl-, 5-quinoline carbonyl-, 6-quinoline carbonyl-, 7-quinoline carbonyl-, 8-quinoline carbonyl-, 1-isoquinoline carbonyl-, 3- isoquinoline carbonyl-, 4- isoquinoline carbonyl-, 5- isoquinoline carbonyl-, 6-isoquinoline carbonyl-, 7- isoquinoline carbonyl-, 8- isoquinoline carbonyl-, 1-benzothiophene carbonyl-, 1-benzofurancarbonyl-, 5-indole-carbonyl-sulfonyl-, N-methylprolinyl-, 2-quinoxaline-carbonyl-, 5-(2,3-dihydrobenzofuran-carbonyl-, 2-benzofurancarbonyl-, 2-benzothiophene-carbonyl-, N-morpholino-carbonyl-, N-methyl-piperidine-carbonyl-, or N-pyrazole-carbonyl-; Het-SO₂, preferably 2-pyridyl sulfonyl-, 3-pyridyl sulfonyl, 4-pyridyl sulfonyl, 2-quinoline sulfonyl-, 3-quinoline sulfonyl-, 4-quinoline sulfonyl-, 5-quinoline sulfonyl-, 6-quinoline sulfonyl-, 7-quinoline sulfonyl-, 8-quinoline sulfonyl-, 1- isoquinoline sulfonyl-, 3- isoquinoline sulfonyl-, 4- isoquinoline sulfonyl-, 5-isoquinoline sulfonyl-, 6- isoquinoline sulfonyl-, 7- isoquinoline sulfonyl-, or 8-isoquinoline sulfonyl-; C₁₋₆ alkyl-CO, preferably acetyl; N,N-dimethyl glycinyl-; C₃₋₁₁cycloalkyl-CO, preferably trans-4-propyl-cyclohexyl-carbonyl-, or cyclohexyl-carbonyl-; C₁₋₆ alkyl-SO₂; C₂₋₆ alkenyl-CO; C₂₋₆ alkenyl-SO₂; C₂₋₆ alkynyl-CO; C₂₋₆ alkynyl-SO₂; ArC₁₋₆ alkyl-CO; ArC₁₋₆ alkyl-SO₂; ArC₂₋₆ alkenyl-CO; ArC₂₋₆ alkenyl-SO₂; Ar—C₂₋₆ alkynyl-CO; Ar—C₂₋₆ alkynyl-SO₂; Het-C₁₋₆ alkyl-CO, preferably 4-imidazole acetyl-, 2-pyridyl acetyl, 3-pyridyl acetyl, 4-pyridyl acetyl-, or N-morpholine acetyl-; Het₁₋₆ alkyl-SO₂; Het-C₂₋₆ alkenyl-CO; Het-C₂₋₆ alkenyl-SO₂; Het-C₂₋₆ alkynyl-CO; or Het-C₂₋₆ alkynyl-SO₂;

and pharmaceutically acceptable salts, hydrates and solvates thereof.

Compounds of Formula II wherein R I,R² or R³ is H are preferred.

Even more preferred are compounds of Formula II wherein:

R¹ is H or C₁₋₆ alkyl, preferably methyl;

R² and R³ are H;

R⁴ is N—(R⁶)—NHCH(C₁₋₆ alkyl)-CO, preferably N—R⁶-leucinyl, more preferably N-(2-pyridyl carbonyl)-leucinyl, N-(8-quinoline carbonyl)-leucinyl, N-(6-quinoline carbonyl)-leucinyl, N-(2-quinoline carbonyl)-leucinyl, N-(4-imidazole acetyl)-leucinyl, N-benzoyl-leucinyl, N-(2-pyridyl sulfonyl)-leucinyl, N-(1-isoquinoline carbonyl)-leucinyl, N-(N-morpholine acetyl)-leucinyl, N-(N-methyl prolinyl)-leucinyl, N-(N, N-dimethyl glycinyl)-leucinyl, N-(8-quinoline sulfonyl)-leucinyl, N-Cbz-leucinyl, N-pentafluorobenzoyl-leucinyl, N-2-naphthoyl-leucinyl, N-1-naphthoyl-leucinyl, N-4-fluorobenzoyl-leucinyl, N-(4-trifluoromethyl benzoyl)-leucinyl N-3,4-difluorobenzoyl-leucinyl, N-3,4-dimethoxybenzoyl-leucinyl, N-(1-benzothiophene-carbonyl)-leucinyL N-(2-benzothiazole-carbonyl)-leucinyl, N-(5-benzothiophene-carbonyl)-leucinyl, N-(6-benzothiophene-carbonyl)-leucinyl, N-(5-indole-carbonyl)-leucinyl, N-(trans-4-propyl cyclohexyl-carbonyl)-leucinyl, N-(2-quinoxaline-carbonyl)-leucinyl, N-5-(2,3-dihydrobenzofuran)-carbonyl)-leucinyl, N-(2-benzofuran-carbonyl)-leucinyl, N-(N-methyl-2-indole-carbonyl)-leucinyl, N-(2-chloro-benzoyl-carbonyl)-leucinyl, N-(4phenoxy-phenyl-carbonyl)-leucinyl, N-(3-methoxy-2-quinoline-carbonyl)-leucinyl, N-(2-pyridyl-methyleneoxy-carbonyl)-leucinyl or N-(cyclobexyl-carbonyl)-leucinyl; or preferably N—R⁶-norleucinyl-, more preferably N-Cbz-norleucinyl, N-(2-naphthyl-carbonyl)-norleucinyl, N-(3,4-dimethoxy-benzoyl)-norleucinyl, or N-(5-benzothiophene-carbonyl)-norleucinyl; or preferably N—R⁶-norvalinyl, more preferably N-Cbz-norvalinyl; or preferably N—R⁶-isoleucinyl, more preferably N-Cbz-isoleucinyl; or preferably N—R⁶-α-allyl-glycinyl; more preferably N-Cbz-α-allyl-glycinyl; or N,N—R⁶-(C₁₋₆ alkyl)-N(C₁₋₆ alkyl)-CO, preferably N,N—R⁶-methyl-leucinyl-, more preferably N-Cbz-N-methyl-leucinyl-; or preferably N—R⁶-α-(cyclopropylmethyl)-glycinyl-, more preferably N-Cbz-α-(cyclopropylmethyl)-glycinyl-; or preferably N—R⁶- L-β-tert-butyl-alaninyl, more preferably N-Cbz-L-β-tert-butyl-alaninyl-or Ar—C₁₋₆ alkyl-CO, preferably 2-(3-biphenyl)-4-methyl-valeryl, or 1-(3-biphenyl)-but-3-ene-1-carbonyl, 1-(3-biphenyl)-ethyl-2-cyclopropane-1-carbonyl;

R⁵ is N—R⁷-norvalinyl-, preferably N-Cbz-norvalinyl-; Ar—C₁₋₆ alkyl-CO, preferably 3-(2-pyridyl)-phenyl acetyl, 3-(3-pyridyl)-phenyl acetyl, 3-(3-biphenyl)-3-methyl-but-3-ene-1-carbonyl, or 2-(3-biphenyl)-but-3-ene-1-carbonyl; or Het-SO₂, preferably 2-pyridyl sulfonyl, 8-quinoline sulfonyl-, 1,3-dimethyl-5-chloro-pyrazole-4-sulfonyl, 3,5-dimethyl-isoxazole-4-sulfonyl, benzo-2,1,3-thiadiazole-4-sulfonyl, or 3-biphenyl sulfonyl; or Het-CO, preferably 8quinolone carbonyl, 5-(2-pyridine) thiophene-carbonyl, N-benzyl-4-piperidinyl carbonyl, 2-quinoline carbonyl or 2-pyridine-carbonyl; or ArCO, preferably 4-phenoxy-phenyl-carbonyl, or 2-(3-biphenyl)-3-methyl-valeryl; Ar—SO₂, preferably 2-carboxymethyl-phenyl-sulfonyl, 2-carboxyl-phenyl-sulfonyl, 4-C-tetrazole-phenyl-sulfonyl, 1-naphthalene-sulfonyl, or 2-cyano-phenyl-sulfonyl; or Ar—CO6 alkyl-, preferably phenyl.

Yet more preferred are compounds of Formula II wherein:

R¹ is H or C₁₋₆ alkyl, preferably methyl;

R² and R³ are H;

R⁴ is N—(R⁶)—NHCH(C₁₋₆ alkyl)-CO, preferably N—R⁶-leucinyl, more preferably Cbz-leucinyl, 2-naphthoyl-leucinyl, 4-fluorobenzoyl-leucinyl, 3,4-dirnethoxybenzoyl-leucinyl, (1-benzothiophene-carbonyl)-leucinyl, (2-quinoxaline-carbonyl)-leucinyl, 5-(2,3-dihydro-benzofuran)-carbonyl)-leucinyl, (2-benzofuran-carbonyl)-leucinyl; or N—R⁶-norleucinyl, more preferably (2-naphthyl-carbonyl)- norleucinyl, (3,4-dimethoxy-benzoyl)-norleucinyl, or (5-benzothiophene-carbonyl)-norleucinyl; or Ar—C₁₋₆ alkyl-CO, preferably 2-(3-biphenyl)-4-methyl-valeryl; and

R⁵ is Ar—C₁₋₆ alkyl-CO, preferably 3-(2-pyridyl)-phenyl acetyl; or Het-SO₂, preferably 2-pyridyl sulfonyl.

Particularly preferred are the compounds of Formula II which are diamino-propan-2-ol analogs of the particularly preferred compounds of Formula I. Most preferred are the compounds of Formula I which are diamino-propan-2-ol analogs of the most preferred compounds of Formula I.

The starting materials used herein are commercially available amino acids or are prepared by routine methods well known to those of ordinary skill in the art and can be found in standard reference books, such as the COMPENDIUM OF ORGANIC SYNTHETIC METHODS, Vol. I-VI (published by Wiley-Interscience).

Coupling methods to form amide bonds herein are generally well known to the art. The methods of peptide synthesis generally set forth by Bodansky et al., THE PRACTICE OF PEPTIDE SYNTHESIS, Springer-Verlag, Berlin, 1984; E. Gross and J. Meienhofer, THE PEPTIDES, Vol. 1, 1-284 (1979); and J. M. Stewart and J. D. Young, SOLID PHASE PEPTIE SYNTHESIS, 2d Ed., Pierce Chemical Co., Rockford, Ill., 1984. are generally illustrative of the technique and are incorporated herein by reference.

Synthetic methods to prepare the compounds of this invention frequently employ protective groups to mask a reactive functionality or minimize unwanted side reactions. Such protective groups are described generally in Green, T. W, PROTECTIVE GROUPS IN ORGANIC SYNTHESIS, John Wiley & Sons, New York (1981). The term “amino protecting groups” generally refers to the Boc, acetyl, benzoyl, Fmoc and Cbz groups and derivatives thereof as known to the art. Methods for protection and deprotection, and replacement of an amino protecting group with another moiety are well known.

Acid addition salts of the compounds of Formula I are prepared in a standard manner in a suitable solvent from the parent compound and an excess of an acid, such as hydrochloric, hydrobromic, hydrofluoric, sulfuric, phosphoric, acetic, trifluoroacetic, maleic, succinic or methanesulfonic. Certain of the compounds form inner salts or zwitterions which may be acceptable. Cationic salts are prepared by treating the parent compound with an excess of an alkaline reagent, such as a hydroxide, carbonate or alkoxide, containing the appropriate cation; or with an appropriate organic amine. Cations such as Li⁺, Na⁺, K⁺, Ca⁺⁺, Mg⁺⁺ and NH₄⁺ are specific examples of cations present in pharmaceutically acceptable salts. Halides, sulfate, phosphate, alkanoates (such as acetate and trifluoroacetate), benzoates, and sulfonates (such as mesylate) are examples of anions present in pharmaceutically acceptable salts.

This invention also provides a pharmaceutical composition which comprises a compound according to Formula I and a pharmaceutically acceptable carrier, diluent or excipient. Accordingly, the compounds of Formula I may be used in the manufacture of a medicament. Pharmaceutical compositions of the compounds of Formula I prepared as hereinbefore described may be formulated as solutions or lyophilized powders for parenteral administration. Powders may be reconstituted by addition of a suitable diluent orther pharmaceutically acceptable carrier prior to use. The liquid formulation may be a buffered, isotonic, aqueous solution. Examples of suitable diluents are normal isotonic saline solution, standard 5% dextrose in water or buffered sodium or ammonium acetate solution. Such formulation is especially suitable for parenteral administration, but may also be used for oral administration or contained in a metered dose inhaler or nebulizer for insufflation. It may be desirable to add excipients such as polyvinylpyrrolidone, gelatin, hydroxy cellulose, acacia, polyethylene glycol, mannitol, sodium chloride or sodium citrate.

Alternately, these compounds may be encapsulated, tableted or prepared in an emulsion or syrup for oral administration. Pharmaceutically acceptable solid or liquid carriers may be added to enhance or stabilize the composition, or to facilitate preparation of the composition. Solid carriers include starch, lactose, calcium sulfate dihydrate, terra alba, magnesium stearate or stearic acid, talc, pectin, acacia, agar or gelatin. Liquid carriers include syrup, peanut oil, olive oil, saline and water. The carrier may also include a sustained release material such as glyceryl monostearate or glyceryl distearate, alone or with a wax. The amount of solid carrier varies but, preferably, will be between about 20 mg to about 1 g per dosage unit. The pharmaceutical preparations are made following the conventional techniques of pharmacy involving milling, mixing, granulating, and compressing, when necessary, for tablet forms; or milling, mixing and filling for hard gelatin capsule forms. When a liquid carrier is used, the preparation will be in the form of a syrup, elixir, emulsion or an aqueous or non-aqueous suspension. Such a liquid formulation may be administered directly p.o. or filled into a soft gelatin capsule.

For rectal administration, the compounds of this invention may also be combined with excipients such as cocoa butter, glycerin, gelatin or polyethylene glycols and molded into a suppository.

### Utility of the Present Invention

The compounds of Formula I are useful as protease inhibitors, particularly as inhibitors of cysteine and serine proteases, more particularly as inhibitors of cysteine proteases, even more particularly as inhibitors of cysteine proteases of the papain superfamily, yet more particularly as inhibitors of cysteine proteases of the cathepsin family, most particularly as inhibitors of cathepsin K. The present invention also provides useful compositions and formulations of said compounds, including pharmaceutical compositions and formulations of said compounds.

The present compounds are useful for treating diseases in which cysteine proteases are implicated, including infections by pneumocystis carinii, trypsanoma cruzi, trypsanoma bmcei, and Crithidia fusiculata; as well as in schistosorniasis, malaria, tumor metastasis, metachromatic leukodystrophy, muscular dystrophy, amytrophy; and especially diseases in which cathepsin K is implicated, most particularly diseases of excessive bone or cartilage loss, including osteoporosis, gingival disease including gingivitis and periodontitis, arthritis, more specifically, osteoarthritis and rheumatoid arthritis, Paget's disease; hypercalcemia of malignancy, and metabolic bone disease.

Metastatic neoplastic cells also typically express high levels of proteolytic enzymes that degrade the surrounding matrix, and certain tumors and metastatic neoplasias may be effectively treated with the compounds of this invention.

The present invention also provides methods of treatment of diseases caused by pathological levels of proteases, particularly cysteine and serine proteases, more particularly cysteine proteases, even more particularly as inhibitors of cysteine proteases of the papain superfamily, yet more particularly cysteine proteases of the cathepsin family, which methods comprise administering to an animal, particularly a mammal, most particularly a human in need thereof a compound of the present invention. The present invention especially provides methods of treatment of diseases caused by pathological levels of cathepsin K, which methods comprise administering to an animal, particularly a mammal, most particularly a human in need thereof an inhibitor of cathepsin K, including a compound of the present invention. The present invention particularly provides methods for treating diseases in which cysteine proteases are implicated, including infections by pneumocystis cariniu, trypsanoma cruzi, trypsanoma brucei, and Crithidia fusiculata; as well as in schistosomiasis, malaria, tumor metastasis, metachromatic leukodystrophy, muscular dystrophy, amytrophy, , and especially diseases in which cathepsin K is implicated, most particularly diseases of excessive bone or cartilage loss, including osteoporosis, gingival disease including gingivitis and periodontitis, arthritis, more specifically, osteoarthritis and rheumatoid arthritis, Paget's disease, hypercalcemia of malignancy, and metabolic bone disease.

This invention further provides a method for treating osteoporosis or inhibiting bone loss which comprises internal administration to a patient of an effective amount of a compound of Formula I, alone or in combination with other inhibitors of bone resorption, such as bisphosphonates (i.e., allendronate), hormone replacement therapy, anti-estrogens, or calcitonin. In addition, treatment with a compound of this invention and an anabolic agent, such as bone morphogenic protein, iproflavone, may be used to prevent bone loss or to increase bone mass.

For acute therapy, parenteral administration of a compound of Formula I is preferred. An intravenous infusion of the compound in 5% dextrose in water or normal saline, or a similar formulation with suitable excipients, is most effective, although an intramuscular bolus injection is also useful. Typically, the parenteral dose will be about 0.01 to about 100 mg/kg; preferably between 0.1 and 20 mg/kg, in a manner to maintain the concentration of drug in the plasma at a concentration effective to inhibit cathepsin K. The compounds are administered one to four times daily at a level to achieve a total daily dose of about 0.4 to about 400 mg/kg/day. The precise amount of an inventive compound which is therapeutically effective, and the route by which such compound is best administered, is readily determined by one of ordinary skill in the art by comparing the blood level of the agent to the concentration required to have a therapeutic effect.

The compounds of this invention may also be administered orally to the patient, in a manner such that the concentration of drug is sufficient to inhibit bone resorption or to achieve any other therapeutic indication as disclosed herein. Typically, a pharmaceutical composition containing the compound is administered at an oral dose of between about 0.1 to about 50 mg/kg in a manner consistent with the condition of the patient. Preferably the oral dose would be about 0.5 to about 20 mg/kg.

No unacceptable toxicological effects are expected when compounds of the present invention are administered in accordance with the present invention.

### Biological Assays

The compounds of this invention may be tested in one of several biological assays to determine the concentration of compound which is required to have a given pharmacological effect.

Determination of Cathepsin K Proteolytic Catalytic Activity

All assays for cathepsin K were carried out with human recombinant enzyme. Standard assay conditions for the determination of kinetic constants used a fluorogenic peptide substrate, typically Cbz-Phe-Arg-AMC, and were determined in 100 mM Na acetate at pH 5.5 containing 20 mM cysteine and 5 MM EDTA. Stock substrate solutions were prepared at concentrations of 10 or 20 mM in DMSO with 20 uM final substrate concentration in the assays. All assays contained 10% DMSO. Independent experiments found that this level of DMSO had no effect on enzyme activity or kinetic constants. All assays were conducted at ambient temperature. Product fluorescence (excitation at 360 nM; emission at 460 nM) was monitored with a Perceptive Biosystems Cytofluor II fluorescent plate reader. Product progress curves were generated over 20 to 30 minutes following formation of AMC product.

Inhibition Studies

Potential inhibitors were evaluated using the progress curve method. Assays were carried out in the presence of variable concentrations of test compound. Reactions were initiated by addition of enzyme to buffered solutions of inhibitor and substrate. Data analysis was conducted according to one of two procedures depending on the appearance of the progress curves in the presence of inhibitors. For those compounds whose progress curves were linear, apparent inhibition constants (K_{i,app}) were calculated according to equation 1 (Brandt et al., Biochemitsry, 1989, 28, 140):

v=VₘA/[Kₐ(1+1/K_{i,app}) +A] (1)

where v is the velocity of the reaction with maximal velocity Vₘ, A is the concentration of substrate with Michaelis constant of Kₐ, and I is the concentration of inhibitor.

For those compounds whose progress curves showed downward curvature characteristic of time-dependent inhibition, the data from individual sets was analyzed to give k_{obs} according to equation 2:

[AMC]=vₛₛt+(v₀−vₛₛ)[1−exp (−k_{obs}t)]/k_{obs} (2)

where [AMC] is the concentration of product formed over time t, v₀ is the initial reaction velocity and vss is the fmal steady state rate. Values for k_{obs} were then analyzed as a linear function of inhibitor concentration to generate an apparent second order rate constant (k_{obs}/inhibitor concentration or k_{obs}/[I]) describing the time-dependent inhibition. A complete discussion of this kinetic treatment has been fully described (Morrison et al., Adv. Enzymol. Relat. Areas Mol. Biol., 1988, 61, 201).

Human Osteoclast Resorption Assay

Aliquots of osteoclastoma-derived cell suspensions were removed from liquid nitrogen storage, warmed rapidly at 37° C. and washed ×1 in RPMI-1640 medium by centrifugation (1000 rpm, 5 min at 4° C.). The medium was aspirated and replaced with murine anti-HLA-DR antibody, diluted 1:3 in RPMI-1640 medium, and incubated for 30 min on ice The cell suspension was mixed frequently.

The cells were washed ×2 with cold RPMI-1640 by centrifugation (1000 rpm, 5 min at 4° C.) and then transferred to a sterile 15 mL centrifuge tube. The number of mononuclear cells were enumerated in an improved Neubauer counting chamber.

Sufficient magnetic beads (5/mononuclear cell), coated with goat anti-mouse IgG, were removed from their stock bottle and placed into 5 mL of fresh medium (this washes away the toxic azide preservative). The medium was removed by immobilizing the beads on a magnet and is replaced with fresh medium.

The beads were mixed with the cells and the suspension was incubated for 30 min on ice. The suspension was mixed frequently. The bead-coated cells were immobilized on a magnet and the remaining cells (osteoclast-rich fraction) were decanted into a sterile 50 mL centrifuge tube. Fresh medium was added to the bead-coated cells to dislodge any trapped osteoclasts. This wash process was repeated ×10. The bead-coated cells were discarded.

The osteoclasts were enumerated in a counting chamber, using a large-bore disposable plastic pasteur pipette to charge the chamber with the sample. The cells were pelleted by centrifugation and the density of osteoclasts adjusted to 1.5×10⁴/mL in EMEM medium, supplemented with 10% fetal calf serum and 1.7 g/litre of sodium bicarbonate. 3 mL aliquots of the cell suspension (per treatment) were decanted into 15 mL centrifuge tubes. These cells were pelleted by centrifugation. To each tube 3 mL of the appropriate treatment was added (diluted to 50 uM in the EMEM medium). Also included were appropriate vehicle controls, a positive control (87MEM1 diluted to 100 ug/mL) and an isotype control (IgG2a diluted to 100 ug/mL). The tubes were incubate at 37° C. for 30 min.

0.5 mL aliquots of the cells were seeded onto sterile dentine slices in a 48-well plate and incubated at 37° C. for 2 h. Each treatment was screened in quadruplicate. The slices were washed in six changes of warm PBS (10 mL/well in a 6Swell plate) and then placed into fresh treatment or control and incubated at 37° C. for 48 h. The slices were then washed in phosphate buffered saline and fixed in 2% glutaraldehyde (in 0.2M sodium cacodylate) for 5 min., following which they were washed in water and incubated in buffer for 5 min at 37° C. The slices were then washed in cold water and incubated in cold acetate buffer/fast red garnet for 5 min at 4° C. Excess buffer was aspirated, and the slices were air dried following a wash in water.

The TRAP positive osteoclasts were enumerated by bright-field microscopy and were then removed from the surface of the dentine by sonication. Pit volumes were determined using the Nikon/Lasertec ILM21W confocal microscope.

### General

Nuclear magnetic resonance spectra were recorded at either 250 or 400 MHz using, respectively, a Bruker AM 250 or Bruker AC 400 spectrometer. CDCl₃ is deuteriochloroform, DMSO-d₆ is hexadeuteriodimethylsulfoxide, and CD₀OD is tetradeuteriomethanol. Chemical shifts are reported in parts per million (d) downfield from the internal standard tetramethylsilane. Abbreviations for NMR data are as follows: s=singlet, d=doublet, t=triplet, q=quartet, m=multiplet, dd=doublet of doublets, dt=doublet of triplets, app=apparent, br=broad. J indicates the NMR coupling constant measured in Hertz. Continuous wave infrared (IR) spectra were recorded on a Perkin-Elmer 683 infrared spectrometer, and Fourier transform infrared (FTIR) spectra were recorded on a Nicolet Impact 400 D infrared spectrometer. IR and FTIR spectra were rded in transmission mode, and band positions are reported in inverse wavenumbers (cm⁻¹). Mass spectra were taken on either VG 70 FE, PE Syx API III, or VG ZAB HF instruments, using fast atom bombardment (FAB) or electrospray (ES) ionization techniques. Elemental analyses were obtained using a Perkin-Elmer 240C elemental analyzer. Melting points were taken on a Thomas-Hoover melting point apparatus and are uncorrected. All temperatures are reported in degrees Celsius.

Analtech Silica Gel GF and E. Merck Silica Gel 60 F-254 thin layer plates were used for thin layer chromatography. Both flash and gravity chromatography were carried out on E. Merck Kieselgel 60 (230-400 mesh) silica gel.

Where indicated, certain of the materials were purchased from the Aldrich Chemical Co., Milwaukee, Wis., Chemical Dynarnics Corp., South Plainfield, N.J., and Advanced Chemtech, Louisville, Ky.

### EXAMPLES

In the following synthetic examples, temperature is in degrees Centigrade (° C.). Unless otherwise indicated, all of the starting materials were obtained from commercial sources. Without further elaboration, it is believed that one skilled in the art can, using the preceding description, utilize the present invention to its fullest extent. These Examples are given to illustrate the invention, not to limit its scope. Reference is made to the claims for what is reserved to the inventors hereunder.

### Example 1

Preparation of 1-N-(N-(2-pyridyl carbonyl)-leucinl)-amino-3-N-(2-pvridyl-sulfonyl)-amino-propan-2-one

a) 1-N-(N-Boc-leucinyl)-amino-3-N-(2-pyridyl-sulfonyl)-amino-propan-2-ol

1,3-Diamino-propan-2-ol (3.375 g, 37.5 mmol) was dissolved in DMF (65 ml). Then HOBT-hydrate (5.5 g, 40.7 mmol), Boc-L-leucine (9.34 g, 37.5 mmol), EDCI (7.77 g, 40.7 mmol), NMM (4.4 ml, 40 mmol) were added, and the reaction mixture was stirred for 4 h; then 2-pyridyl-sulfonyl chloride (3.7 g, 20.8 mmol) was added reaction was stirred an additional 2 h. The reaction mixture was concentrated in vacuo, then cbromatographed on silica gel to yield a white solid (4.3 g, 26%) (ES+) 445.2 (M+H⁺).

b) 1-N-(leucinyl)-amino-3-N-(2-pyridyl-sulfonyl)-amino-propan-2-ol

1-N-(N-Boc-leucinyl)-amino-3-N-(2-pyridyl-sulfonyl)-amino-propan-2-ol (2.1 g, 4.73 mmol) was dissolved in 1:1 TFA: DCM (60 ml) and was stirred at RT for 1 h. Toluene (100 ml) was added then the reaction mixture was concentrated in vacuo and was used in the following reaction without further purification (1.6 g, quant.).

c) 1-N-(N-(2-pyridyl carbonyl)-leucinyl)-amino-3-N-(2-pyridyl-sulfonyl)-amino-propan-2-ol

HBTU (0.6 g, 1.6 mmol) was added to a solution of 1-N-(leucinyl)-amino-3-N-(2-pyridyl-sulfonyl)-amino-propan-2-ol (0.9 g, 1.58 mmol), NMM (0.87 ml, 8 nmmol), and 2-pyridine carboxylic acid (0.194 g, 1.58 mmol) in DMF (11.5 ml). The reaction mixture was stirred overnight, then was washed with brine/EtOAc, 1 N NaOH; the combined organics were dried with MgSO4, filtered, concentrated, and was used in the next reaction without further purification: MS(ES) (ES+) 450.1 (M+H⁺).

d) 1-N-(N-(2-pyridyl carbonyl)-leucinyl)-amino-3-N-(2-pyidyl-sulfonyl)-aniino-propan-2-one

1-N-(N-(2-pyridyl carbonyl)-leucinyl)-amino-3-N-(2-pyridyl-sulfonyl)-amino-propan-2-ol (from Example 1c) was dissolved in acetone (10 ml), then 1N HCl (5 ml) in ether was added dropwise, then the solution was concentrated in vacuo. The solid was redissolved in acetone (10 ml), then Jones reagent (1N, 1 ml) was added dropwise and the reaction was stirred overnight. The reaction was quenched with isopropanol (1 ml), then The reaction mixture was basified with 1N NaOH, and was then extracted repeatedly with EtOAc. The combined organics were dried with MgSO4, filtered, concentrated, and chromatographed on silica gel to yield a white solid (109 mg, 15.4%, 2 steps): MS (ES+) 448.1 (MH⁺), 470.2 (M+Ha⁺).

### Example 2

Preparation of 1-N-(N-(8-quinoline carbonyl)-leucinyl)-amino-3-N-(2-pyridyl-sulfonyl)-amino-propan-2-one

a) 1-N-(N-(8-quinoline carbonyl)-leucinyl)-amino-3-N-(2-pyridyl-sulfonyl)-amino-propan-2-one

Following the procedure of Example 1 (a-d), except substituting “8-quinoline carboxylic acid” for “2-pyridine carboxylic acid”, the tide compound was prepared: MS (ES+) 498.3 (M+H⁺).

### Example 3

Preparation of 1-N-(N-(2-quinoline carbonyl)-leucinyl)-amino-3-N-(2-pyridyl-sulfonyl)-amino-propan-2-one

a) 1-N-(N-(2-quinoline carbonyl)-leucinyl)-amino-3-N-(2-pyridyl-sulfonyl)-amino-propan-2-one

Following the procedure of Example 1 (a-d), except substituting “2-quinoline carboxylic acid” for “2-pyridine carboxylic acid”, the title compound was prepared: MS (ES+) 498.1 (M+H⁺).

### Example 4

Preparation of 1-N-(N-(4-imidazole acetyl)-leucinyl)-amino-3-N-(3-biphenyl sulfonyl)-amino-propan-2-one

a) 1-N-(N-(4-imidazole acetyl)-leucinyl)-amino-3-N-(3-biphenyl sulfonyl)-amino-propan-2-one

Following the procedure of Example 1 (a-d), except substituting “4-imidazole carboxylic acid” for “2-pyridine carboxylic acid” and “3-biphenyl sulfonyl chloride” for “2-pyridyl sulfonyl chloride”, the title compound was prepared: MS (ES+) 526.3 (M+H⁺).

### Example 5

Preparation of 1-N-(N-(2-pyridyl-carbonyl)-leucinyl)-amino-3-N-(8-quinoline carbonyl)-amino-propan-2-one

a) 1-N-(N-(2-pyridyl-carbonyl)-leucinyl)-amino-3-N-(8-quinoline carbonyl)-amino-propan-2-one

Following the procedure of Example 1 (a-d), except substituting “8-quinoline carboxylic acid and EDCI” for “2-pyridyl sulfonyl chloride”, the title compound was prepared: MS (ES+) 462.2 (M+H⁺), 484.2 (M+Ha⁺).

### Example 6

Preparation of 1-N-(N-benzoyl-leucinyl)-amino-3-N-(8-guinoline carbonyl-amino-propan-2-one

a) 1N-(N-benzoyl-leucinyl)-amino-3N-(8-quinoline carbonyl)-amino-propan-2-one

Following the procedure of Example 5, except substituting “benzoic acid” for “2-pyridine carboxylic acid”, the title compound was prepared: MS (ES+) 461.3 (M+H⁺), 483.2 (M+Ha⁺).

### Example 7

Preparation of 1-N-(N-(2-pyridyl sulfonyl)-leucinyl)-amino-3-N-(8-guinoline carbonyl)-amino-propan-2-one

a) 1-N-(N-(2-pyridyl sulfonyl)-leucinyl)-amino-3-N-(8-quinoline carbonyl)-amino-propan-2-one

Following the procedure of Example 5, except substituting “2-pyridine sulfonyl chloride” for “2-pyridine carboxylic acid and HBTU”, the title compound was prepared: MS (ES+) 498.2 (M+H⁺).

### Example 8

Preparation of 1-N-(N-(8-guinoline carbonyl)-leucinyl)-amino-3-N-(8-guinoline carbonyl)-amino-propan-2-one

a) 1-N-(N-(8-quinoline carbonyl)-leucinyl)-amino-3-N-(8-quinoline carbonyl)-amino-propan-2-one

Following the procedure of Example 5, except substituting “8-quinoline carboxylic acid” for “2-pyridine carboxylic acid”, the title compound was prepared: MS (ES+) 512.3 (M+H⁺), 534.2 (M+Ha⁺).

### Example 9

Preparation of 1-N-(N-(1-isoguinoline-carbonyl)-leucinyl)-amino-3-N-(8-quinoline carbonyl)-amino-propan-2-one

a) 1-N-(N-(1-isoquinoline-carbonyl)-leucinyl)-amino-3-N-(8-quinoline carbonyl)-amino-propan-2-one

Following the procedure of Example 5, except substituting “1-isoquinoline carboxylic acid” for “2-pyridine carboxylic acid”, the title compound was prepared: MS (ES+) 512.4 (M+H⁺), 534.1 (M+Ha⁺).

### Example 10

Preparation of 1-N-(N-(N-morpholine-acetyl)-leucinyl)-amino-3-N-(8-guinoline carbonyl)-amino-propan-2-one

a) 1-N-(N-(N-morpholine-acetyl)-leucinyl)-amino-3-N-(8-quinoline carbonyl)-amino-propan-2-one

Following the procedure of Example 5, except substituting “N-morpholine acetic acid” for “2-pyridine carboxylic acid”, the title compound was prepared: MS (ES+) 484.3 (M+H⁺).

### Example 11

Preparation of 1-N-(N-(N-methyl prolinyl)-leucinyl)-amino-3-N-(8-guinoline carbonyl)-amino-pro2an-2-one

a) 1-N-(N-(N-methyl prolinyl)-leucinyl)-amino-3-N-(8-quinoline carbonyl)-amino-propan-2-one

Following the procedure of Example 5, except substituting “N-methyl proline” for “2-pyridine carboxylic acid”, the title compound was prepared: MS (ES+) 468.2 (M+H⁺).

### Example 12

Preparation of 1-N-(N-(N,N-dimethyl glycinyl)-leucinyl)-amino-3-N-(8-guinoline carbonyl)-amino-propan-2-one

a) 1-N-(N-(N, N-dimethyl glycinyl)-leucinyl)-amino-3-N-(8-quinoline carbonyl)-amino-propan-2-one

Following the procedure of Example 5, except substituting “N, N-dimethyl glycine” for “2-pyridine carboxylic acid”, the title compound was prepared: MS (ES+) 442.1 (M+H⁺).

### Example 13

Preparation of 1-N-(N-(8-quinoline sulfonyl)-leucinyl)-amino-3-N-(8-quinoline carbonyl)-amino-propan-2-one

a) 1-N-(N-(8-quinoline sulfonyl)-leucinyl)-amino-3-N-(8-quinoline carbonyl)-amino-propan-2-one

Following the procedure of Example 5, except substituting “8-quinoline sulfonyl chloride” for “2-pyridine carboxylic acid and HBTU”, the title compound was prepared: MS (ES+) 548.3 (M+H⁺).

### Example 14

Preparation of 1-N-(N-Cbz-leucinyl)-amino-3-N-(3-(2-pyridyl)-phenyl acetyl)-amino-propan-2-one

a) 3-(trifluoromethyl sulfonyloxy)-phenyl acetic acid methyl ester

To an oven-dried flask under Argon atmosphere containing sodium hydride (2.54 g, 60% dispersion in mineral oil, 63.5 nimol) was added anhydrous pentane (20 mL). The slurry was stirred for 5 min, allowed to settle, most of the pentane was removed, and anhydrous THF (40 mL) was added. To this suspension was added a solution of 3-hydroxyphenylacetic acid methyl ester (9.99 g, 60.1 mmol) in anhydrous THF(20 mL) and the reaction was stirred at room temperature for 20 min. To this mixture was then added a solution of N-phenyltrifluoromethanesulfonimide (22.53 g, 63.1 mmol)) in anhydrous THF (40 mL) and the reaction was stirred at room temperature until TLC analysis indicated the complete consumption of starting material (1.5 h). The reaction was quenched by the addition of H₂O (10 mL), concentrated to one half original volume, then diluted with CHCl₃ (200 mL) and washed with H₂O. The aqueous layer was washed with fresh CHCl₃ (50 mL), the combined organic layers were washed with 10% Na₂CO₃, H₂O, and brine, then dried (MgSO4), filtered and concentrated. Column chromatography of the residue (silica gel, 5:95 EtOAc: hexanes, then 10:90 EtOAc: hexanes) gave 17.47 g of the title compound: ¹H NMR (400 MHz, CDCl₃) 7.42 (m, 1H), 7.31-7.19 (m, 3H), 3.72 (s, 3H), 3.68 (s, 2H).

b) 3-(2-pyridyl)-phenyl Acetic Acid Methyl Ester

To a solution of the compound of 3-(trifluoromethyl sulfonyloxy)-phenyl acetic acid methyl ester (6.86 g, 23.0 mmol) in anhydrous dioxane (100 mL) was added 2-pyridylstannane (8.89 g, 24.1 mmol), LiCl (2.94 g, 69.3 mmol), 2,6-di-tert-butyl-4-methylphenol (a few crystals), and Pd(PPh₃)₄ (632.1 mg, 0.55 mmol). The reaction was protected from light with foil and heated to reflux overnight. The reaction was allowed to cool to room temperature and concentrated. Column chromatography of the residue (silica gel, 1:3 EtOAc: hexanes, then 1:2 EtOAc: hexanes) gave 3.85 g of the title compound: MS(ES⁺) 228.1 (MH⁺).

c) 3-(2-pyridyl)-phenyl Acetic Acid

To a solution of the compound of 3-(2-pyridyl)-phenyl acetic acid methyl ester (3.8 g, 16.7 mmol) in THF (50 mL) was added a solution of LiOHH₂O (780.2 mg, 18.6 mmol) in H₂O (10 mL). The reaction was stirred at room temperature until TLC analysis indicated the complete consumption of starting material (2 h). The reaction mixture was concentrated to remove THF, then neutralized to pH=7 by the addition of 1N HCl, diluted with brine (50 mL), and washed with CHCl₃ (100 mL) The aqueous layer was readjusted back to pH=7 by the addition on 1N NaOH and washed with fresh CHCl₃ (100 mL). After repeating this procedure once more, the organic layers were combined, dried, filtered (MgSO₄) and concentrated to give 3.79 g of the title compound: MS (ES⁺) 214.3 (MH⁺).

d) 1-N-(N-Cbz-leucinyl)-amino-3-N-(3-(2-pyridyl)-phenyl acetyl)-amino-propan-2-ol

Following the procedure of Example 1 (a-c), except substituting “Cbz-leucine” for “Boc-Leucine” and “3-(2-pyridyl)-phenyl acetic acid and EDCI” for “2-pyridyl sulfonyl chloride” the title compound was prepared: MS (ES+) 533.3 (M+H⁺).

e) 1-N-(N-Cbz-leucinyl)-amino-3-N-(3-(2-pyridyl)-phenyl acetyl)-amino-propan-2-one

Following the procedure of Example 1 (d), except substituting “1-N-(N-Cbz-leucinyl)-amino-3-N-(3-(2-pyridyl)-phenyl acetyl)-amino-propan-2-ol” for “1-N-(N-2-pyridyl carbonyl-leucinyl)-amino-3-N-(2-pyridyl-sulfonyl)-amnino-propan-2-ol ”, the title compound was prepared: MS (ES+) 531.4 (M+H⁺).

### Example 15

Preparation of 1-N-(N-pentafluorobenzoyl-leucinyl)-amino-3-N-(3-(2-pryridyl)-phenyl acetyl)-amino-propan-2-one

a) leucinyl-amino-3-N-(3-(2-pyridyl)-phenyl acetyl)-amino-propan-2-ol

1-N-(N-Cbz-leucinyl)-amino-3-N-(3-(2-pyridyl)-phenyl acetyl)-amino-propan-2-ol (Example 1d, 5.5 g, 11.4 mmol) was dissolved in EtOH (100 ml), then 10% Pd/C (1.1 g, mmol) was added and the solution was hydrogenated on a Parr shaker at 50 atmospheres for 12 h. The reaction mixture was filtered through Celite, concentrated in vacuo, then was used in the next reaction without further purification (3.5 g, quant.): MS (ES+) 303.2 (MH⁺).

b) 1-N-(N-pentafluorobenzoyl-leucinyl)-amino-3-N-(3-(2-pyridyl)-phenyl acetyl)-amino-propan-2-ol

HBTU (0.2 g, 0.53 mmol) was added to a solution of leucinyl-amino-3-N-(3-(2-pyridyl)-phenyl acetyl)-amino-propan-2-ol (0.23 g, 0.58 mmol), pentaflurobenzoic acid (0.106 g, 0.5 mmol), NMM (0.23 ml, 2 mmol) in DMF (5 ml) and was stirred overnight. The reaction mixture was poured into water, extracted with EtOAc; the organic layer was dried with MgSO₄, filtered, concentrated in vacuo, and chromatographed on silica gel to yield a white solid (0.146 g, 50%): MS (ES+) 595.1 (MH⁺).

c) 1-N-(N-pentafluorobenzoyl-leucinyl)-amino-3-N-(3-(2-pyridyl)-phenyl acetyl)-amino-propan-2-one

Dess-Martin periodinane (J. Org. Chem. 1983, 48, 4155-4156, 0.12 g, 0.28 mmol) was added to a solution of 1-N-(N-pentafluorobenzoyl-leucinyl)-amino-3-N-(3-(2-pyridyl)-phenyl acetyl)-amino-propan-2-ol (0.146 g, 0.25 mmol) in CH₂Cl₂ (40 ml) and was stirred for 3 h. The reaction was diluted with 50 ml CH₂Cl₂, then 10% aqueous Na₂S₂O₃ (10 ml) and aq. 10% NaHCO₃ (10 ml) was added and the reaction was stirred for 10 min. The organic layer was dried with MgSO4, filtered, concentrated in vacuo, and chromatographed on silica gel to yield a white solid (44 mg, 30%): MS (ES+) 593.1 MH⁺).

### Example 16

Preparation of 1-N-(N-2-naphthoyl-leucinyl)-amino-3-N-(3-(2-pyridyl)-phenyl acetyl)-amino-propan-2-one

a) 1-N-(N-2-naphthoyl-leucinyl)-amino-3-N-(3-(2-pyridyl)-phenyl acetyl)-amino-propan-2-one

Following the procedure of Example 15 (a-c), except substituting “2-naphthoic acid” for “pentafluorobenzoic acid”, the title compound was prepared: MS (ES+) 551.2 (M+H⁺).

### Example 17

Preparation of 1-N-(N-1-naphthoyl-leucinyl)-amino-3-N-(3-(2-pyridyl)-phenyl acetyl-amino-propan-2-one

a) 1-N-(N-1-naphthoyleucinyl)-amino-3-N-(3-(2-pyridyl)-phenyl acetyl)-amino-propan-2-one

Following the procedure of Example 15 (a-c), except substituting “1-naphthoic acid” for “pentafluorobenzoic acid”, the title compound was prepared: MS (ES+) 551.1 (M+H⁺).

### Example 18

Preparation of 1-N-(N-(2-pyridyl-carbonyl)-leucinyl)-amino-3-N-(3-(2-pyridyl)-phenyl acetyl)-amino-propan-2-one

a) 1-N-(N-(2-pyridyl-carbonyl)-leucinyl)-amino-3-N-(3-(2-pyridyl)-phenyl acetyl)-amino-propan-2-one

Following the procedure of Example 15 (a-c), except substituting “2-pyridine carboxylic acid” for “pentafluorobenzoic acid”, the title compound was prepared: MS (ES+) 502.3 (M+H⁺).

### Example 19

Preparation of 1-N-(N-4-fluorobenzoyl-leucinyl)-amino-3-N-(3-(2-pyridyl)-phenyl acetml)-amino-propan-2-one

a) 1-N-(N-4-fluorobenzoyl-leucinyl)-amino-3-N-(3-(2-pyridyl)-phenyl acetyl)-amino-propan-2-one

Following the procedure of Example 15 (a-c), except substituting “4-fluorobenzoic acid” for “pentafluorobenzoic acid”, the title compound was prepared: MS (ES+) 519.4 (M+H⁺), 541.4 (M+Ha⁺).

### Example 20

Preparation of 1-N-(N-3,4-difluorobenzoyl-leucinyl)-amino-3-N-(3-(2-pyridyl)-phenyl acetyl)-amino-propan-2-one

a) 1-N-(N-3,4difluorobenzoyl-leucinyl)-amino-3-N-(3-(2-pyridyl)-phenyl acetyl)-amino-propan-2-one

Following the procedure of Example 15 (a-c), except substituting “3,4-difluorobenzoic acid” for “pentafluorobenzoic acid”, the title compound was prepared: MS (ES+) 537.2 (M+H⁺), 559.2 (M+Ha⁺).

### Example 21

Preparation of 1-N-(N-3,4-dimethoxybenzoyl-4-leucinyl)-amino-3-N-(3-(2-pyridyl)-phenyl acetyl)-amino-propan-2-one

a) 1-N-(N-3,4dimethoxybenzoyl-leucinyl)-amino-3-N-(3-(2-pyridyl)-phenyl acetyl)-amino-propan-2-one

Following the procedure of Example 15 (a-c), except substituting “3,4-dimethoxybenzoic acid” for “pentafluorobenzoic acid”, the title compound was prepared: MS (ES+) 561.2 (M+H⁺), 593.2 (M+Ha⁺).

### Example 22

Preparation of 1-N-(N-1-(benzothiophene-carbonyl)-leucinyl)-amino-3-N-(3-(2-pyridyl)-phenyl acetyl)-amino-pnopan-2-one

a) 1-N-(N-1-benzothiophene-carbonyl-leucinyl)-amino-3-N-(3-(2-pyridyl)-phenyl acetyl)-amino-propan-2-one

Following the procedure of Example 15 (a-c), except substituting “benzothiophene-carboxylic acid” for “pentafluorobenzoic acid”, the title compound was prepared: MS (ES+) 557.2 (M+H⁺).

### Example 23

Preparation of 1-N-(N-(5-indole-carbonyl)-leucinyl)-amino-3-N-(3-(2-pvridyl)-phenyl acetyl)-amino-propan-2-one

a) 1-N-(N-(5-indole-carbonyl)-leucinyl)-amino-3-N-(3-(2-pyridyl)-phenyl acetyl)-amino-propan-2-one

Following the procedure of Example 15 (a-c), except substituting “5-indole-carboxylic acid” for “pentafluorobenzoic acid”, the title compound was prepared: MS (ES+) 540.2 (M+H⁺).

### Example 24

Preparation of 1-N-(N-Cbz-isoleucinyl)-amino-3-N-(3-(2-pyfidyl)-phenyl acetyl)-amino-propan-2-one

a) 1-N-(N-Cbz-isoleucinyl)-amino-3-N-(3-(2-pyridyl)-phenyl acetyl)-amino-propan-2-one

Following the procedure of Example 14 (a-e), except substituting Cbz-isoleucine” for “Cbz-leucine”, the title compound was prepared: MS (ES+) 531.1 (M+H⁺), 553.1 (M+Ha⁺).

### Example 25

Preparation of 1-N-(N-Cbz-norvalinyl)-amino-3-N-(3-(2-pyridyl)-phenyl acetyl)-amino-propan-2-one

a) 1-N-(N-Cbz-valinyl)-amino-3-N-(3-(2-pyridyl)-phenyl acetyl)-amino-propan-2-one

Following the procedure of Example 14 (a-e), except substituting “Cbz-no-valine” for “Cbz-leucine”, the title compound was prepared: MS (ES+) 517.2 (M+H⁺).

### Example 26

Preparation of 1-N-(N-Cbz-α-allyl-glycinyl)-amino-3-N-(3-(2-pyridyl)-phenyl acetyl)-amino-propan-2-one

a) 1-N-(N-Cbz-α-allyl-glycinyl)-amino-3-N-(3-(2-pyridyl)-phenyl acetyl)-amino-propan-2-one

Following the procedure of Example 14 (a-e), except substituting “Cbz-α-allyl-glycine” for “Cbz-leucine”, the title compound was prepared: MS (ES+) 517.2 (M+H⁺).

### Example 27

Preparation of 1-N-(N-Cbz-norleucinyl)-amino-3-N-(3-(2-pyridyl)-phenyl acetyl)-amino-propan-2-one

a) 1-N-(N-Cbz-norleucinyl)-amino-3-N-(3-(2-pyridyl)-phenyl acetyl)-amino-propan-2-one

Following the procedure of Example 14 (a-e), except substituting “Cbz-norleucine” for “Cbz-leucine”, the title compound was prepared: MS (ES+) 531.3 (M+H⁺).

### Example 28

Preparation of 1-N-(N-Cbz-N-methyl-leucinyl)-amino-3-N-(3-(2-pyridyl)-phenyl acetyl)-amino-propan-2-one

a) 1-N-(N-Cbz-N-methyl-leucinyl)-amino-3-N-(3-(2-pyridyl)-phenyl acetyl)-amino-propan-2-one

Following the procedure of Example 14 (a-e), except substituting “Cbz-N-methyl-leucine” for “Cbz-leucine”, the title compound was prepared: MS (ES+) 545.3 (M+H⁺).

### Example 29

Preparation of 1-N-(N-Cbz-α-(cycloproply-methyl-glycinyl)-amino-3-N-(3-(2-pyridyl)-phenyl acetyl)-amino-propan-2-one

a) N-Cbz-α-(cyclopropyl)-methyl-glycine methyl ester

Diazomethane (4.8 mmol in 18 ml Et₂O) was added to a solution of N-Cbz-L-α-allyl-glycine (0.210 g, 0.48 mmol) in 1 ml Et₂O at RT and was stirred for 5 minutes. Then Pd(OAc)₂ was added and the reaction was stirred overnight, filtered through silica gel, concentrated in vacua, and was used in the next reaction without further purification (205 mg, 95% yield): MS (ES+) 300.1 (M+Ha⁺).

b) N-Cbz-α-(cyclopropyl)-methyl-glycine

N-Cbz-α-(cyclopropyl)-methyl-glycine methyl ester (205 mg, 0:75 mmol) was dissolved in MeOH (5 ml), then 1N NaOH (0.75 ml) was added dropwise and the reaction was stirred at RT for 12 h. The reaction mixture was diluted with AcOH, extracted with EtOAc, dried with MgSO4, filtered, concentrated in vacuo, and chromatographed (silica gel, 3% MeOH—CH₂Cl₂) to give the title compound as a white solid (165 mg, 82%): MS (ES⁺) 264.2 (M+H⁺), 286.3 (M+Ha⁺), 549.2 (2M+Ha⁺).

c) 1-N-(N-Cbz-α-(cyclopropyl)-methyl-glycinyl)-amino-3-N-(3-(2-pyridyl)-phenyl acetyl)-amino-propan-2-one

Following the procedure of Example 14 (a-e), except substituting “N-Cbz-α-(cyclopropyl)-methyl-glycine” for “Cbz-leucine”, the title compound was prepared: MS (ES+) 529.3 (M+H⁺), 551.4 (M+Ha⁺).

### Example 30

Preparation of 1-N-(N-benzyloxycarbonyl-L-β-tert-butylalanine)-amino-3-N-(3-(2-pyridyl)-phenyl acetyl-amino-propan-2-one

a) N-benzyloxycarbonyl-L-β-tert-butylalanine

To a stirring solution of L-β-tert-butylalanine (1.0 g, 6.89 mmol) in water (2.1 mL) and S N NaOH (1.38 mL) at 0° C. was added benzyl chloroformate (1.3 g, 7.58 mmol) and 2 N NaOH (3.8 mL) in ten alternating portions, over 1.5 h. After the additions were complete the mixture was stirred for another 30 min. at room temperature. The pH was then taken to 10 and the mixture is extracted with ether (50 mL). The aqueous layer was acidified to pH 3 with 3 N HCl and extracted with ether (3×50 mL). The organic layers were combined, dried (MgSO₄), filtered and concentrated to yield the title compound as a colorless oil (1.59 g, 83%). MS(ESI): 278.2 (M+H)⁻.

b) 1-N-(N-benzyloxycarbonyl-L-β-tert-butylalanine)-amino-3-N-(3-(2-pyridyl)-phenyl acetyl)-amino-propan-2-one

Following the procedure of Example 14 (a-e), except substituting “N-benzyloxycarbonyl-L-β-tert-butylalanine” for “Cbz-leucine”, the title compound was prepared: MS (ES+) 545.2 (M+H⁺), 567.3 (M+Ha⁺).

### Example 31

Preparation of 1-N-(2-(3-biphenyl)-4-methyl-valeryl)-amino-3-N-(2-pyridyl-sulfonyl)-amino-propan-2-one

a) 3-bromo-phenyl methyl acetate

3-Bromo phenyl acetic acid (2.15 g, 10 mmol) was dissolved in ether, then was treated with a solution of diazomethane until the yellow color persisted. The reaction was then quenched with AcOH, concentrated in vacuo and was used in the next reaction without further purification.

b) 3-biphenyl methyl acetate

3-bromcphenyl methyl acetate (2.29 g, 10 mmol) was dissolved in toluene (30 ml). Then, phenyl boronic acid (1.46 g, 12 mmol) was added followed by aqueous sodium carbonate (2M, 4.24 ml, 40 mmol), then tetrakis(triphenylphosphine) palladium (0.35 g, 0.3 mmol) and was refluxed overnight. The reaction was cooled to RT, diluted with saturated arammonium chloride, then extracted with EtOAc (2×10 ml). The combined organics were dried with magnesium sulfate, filtered, concentrated, and chromatographed (silica gel, 5% EtOAc: hexanes) to provide the desired product as a white solid (1.93 g, 84%): MS(ES): M+H⁺=263.

c) 3-biphenyl acetic acid

3-Biphenyl acetyl methyl ester was dissolved in MeOH (40 ml) and water (6 ml), then LiOH-hydrate (0.7 g, 16.8 mmol) was added, and the reaction was stirred at RT for 2 h. The reaction was diluted with water, acidified with 6N hydrochloric acid (1 ml), then with EtOAc (2×10 ml). The combined organics were dried with magnesium sulfate, filtered, and concentrated to give the desired product as a white solid (1.66 g, 93%): ¹H NMR: d: 7.6-7.25 (m, 9H), 3.7 (s, 2H)

d) 2-(3-biphenyl)-4-methyl-pent-4-enoic acid

nBuLi (3.26 ml, 1.6 M in hexanes) was added dropwise to a solution of diisopropyl amine (0.74 ml, 5.3 mmol) in THF (6 ml) at 0 C. The reaction was stirred for 15 minutes, then was cooled to −78 C. 3-Biphenyl acetic acid (0.5 g, 2.35 mmol) wasa dissolved in THF (2 ml) and was added dropwise to the LDA solution. The reaction was warmed to 0 C., stirred 40 minutes, then cooled to −78 C. Isobutenyl bromide (0.475 g, 3.52 mmol) was added and the reaction was stirred for 1 h. Water (2 ml) was added and the ThF was removed in vacuo. The reaction was diluted with water, acidified with 6N hydrochloric acid (1 ml), then with EtOAc (2×10 ml). The combined organics were dried with magnesium sulfate, filtered, concentrated, chromatographed (silica gel, 5% MeOH: methylene chloride) to give the desired product as a white solid (1.66 g, 93%): 1H NMR: d: 7.6-7.3 (m, 9H), 4.75 (d, 2H), 3.87 (t, 1H), 2.87 (dd, 1H), 2.50 (dd, 1H), 1.70 (s, 3H).

e) 2-(3-biphenyl)-4-methyl-pentanoic acid

2-(3-Biphenyl)-4-methyl-pent-4-enoic acid (0.5 g, 1.87 mmol) was dissolved in EtOAc (25 ml). Then, 10% Pd/C (60 mg) was added and the reaction was stirred for 2.5 h under a balloon of hydrogen gas. The reaction was filtered, concentrated in vacuo, then was redissolved in 1:5 EtOAc: EtOH (15 ml). Then, 10% Pd/C (80 mg) was added and the reaction was stirred under a balloon of hydrogen gas overnight. The reaction was filtered, concentrated in vacuo, and chromatographed (silica gel, 5% MeOH: methylene chloride) to give the desired product as a white solid (1.66 g, 93%): 1H NMR: d: 7.6-7.3 (m, 9H), 3.7 (t, 1H), 2.07-1.95 (m, 1H), 1.8-1.7 (m, 1H), 1.6-1.45 (m, 1H).

f) 1-N-(2-(3-biphenyl)-4-methyl-valeryl)-amino-3-N-(2-pyridyl-sulfonyl)-amino-propan-2-one

Following the procedure of Example 1 (a) and (d), except substituting “3-(4-biphenyl)-4methyl-pentanoic acid” for “Boc-leucine”, the title compound was prepared: MS (ES+) 480.2 (M+H⁺).

### Example 32

Preparation of 1-N-(2-(3-biphenyl)-4-methyl-valeryl)-amino-3-N-(2-carboxymethyl-phenyl-sulfonyl)-amino-propan-2-one

a) 1-N-(2-(3-biphenyl)-4methyl-valeryl)-amino-3-N-(2-carboxymethyl-phenyl-sulfonyl)-amino-propan-2-one

Following the procedure of Example 31 (a-f), except substituting “2-carboxymethyl-phenyl sulfonyl chloride” for “2-pyridyl sulfonyl chloride”, the title compound was prepared: MS (ES+) 537.1 (M+H⁺), 559.1 (M+Ha⁺), 1073.5 (2M+H⁺), 1095.3 (2M+Ha⁺).

### Example 33

Preparation of 1-N-(2-(3-biphenyl)-4-methyl-valeryl)-amino-3-N-(4-cyano-phenyl-sulfonyl)-amino-propan-2-one

a) 1-N-(2-(3-biphenyl)-4-methyl-valeryl)-amino-3-N-(4-cyano-phenyl-sulfonyl)-aminopropan-2-one

Following the procedure of Example 31 (a-f), except substituting “4-cyano-phenyl sulfonyl chloride” for ““2-pyridyl sulfonyl chloride”, the title compound was prepared: MS (ES+) 504.3 (M+H⁺).

### Example 34

Preparation of 1-N-(2-(3-biphenyl)-4-methyl-valerl)-amino-3-N-(8-guinoline carbonyl)-amino-propan-2-one

a) 1-N-(2-(3-biphenyl)-4-methyl-valeryl)-amino-3-N-(8-quinoline carbonyl)-amino-propan-2-one

Following the procedure of Example 31 (a-f), except substituting “8-quinoline carboxylic acid and EDCI” for ““2-pyridyl sulfonyl chloride”, the title compound was prepared: MS (ES+) 494.2 (M+H⁺).

### Example 35

Preparation of 1-N-(2-(3-biphenyl)-4methyl-valeryl)-amino-3-N-(3-2-pyridyl)-phenyl acetyl)-amino-propan-2-one

a) 1-N-(2-(3-biphenyl)-4-methyl-valeryl)-amino-3-N-(3-(2-pyridyl)-phenyl acetyl)-amino-propan-2-one

Following the procedure of Example 34 (a), except substituting “3-(2-pyridyl)-phenyl acetic acid” for ““8-quinoline carboxylic acid”, the title compound was prepared: MS (ES+) 534.3 fM+H⁺).

### Example 36

Preparation of 1-N-(2-(3-biphenyl)-4-methyl-valeryl)-amino-3-N-(3-(3-2yridyl)-3-phenyl acetyl)-amino-propan-2-one

a) 1-N-(2-(3-biphenyl)-4-methyl-valeryl)-amino-3-N-(3-(3-pyridyl)-3-phenyl acetyl)-amino-propan-2-one

Following the procedure of Example 34 (a), except substituting “3-(3-pyridyl)-phenyl acetic acid” for ““8-quinoline carboxylic acid”, the title compound was prepared: MS (ES+) 534.3 (M+H⁺).

### Example 37

Preparation of 1-N-2-(3-biphenyl)-4-methyl-valeryl)-amino-3-N-(2-pyridine carbonyl)-amino-propan-2-one

a) 1-N-(2-(3-biphenyl)-4methyl-valeryl)-amino-3-N-(2-pyridine carbonyl)-amino-propan-2-one

Following the procedure of Example 34 (a), except substituting “2-pyridine carboxylic acid” for ““8-quinoline carboxylic acid”, the title compound was prepared: MS (S+) 444.3 (M+H⁺).

### Example 38

Preparation of 1-N-(2-(3-biphenyl 4methyl-valeryl)-amino-3-N-(5-(2-pyridine)-thiophene-carbonyl)-amino-propan-2-one

a) 1-N-(2-(3-biphenyl)-4-methyl-valeryl)-amino-3-N-(5-(2-pyridine)-thiophene-carbonyl)-amino-propan-2-one

Following the procedure of Example 34 (a), except substituting “5-(2-pyridine)-thiophene-carboxylic acid” for ““8-quinoline carboxylic acid”, the title compound was prepared: MS (ES+) 526.3 (M+H⁺), 1051.3 (2M+H⁺).

### Example 39

Preparation of 1-N-(2-(3-biphenyl)-4-methyl-valeryl)-amino-3-N-(N-benzyl-4piperidine-carbonyl)-amino-propan-2-one

a) 1-N-(2-(3-biphenyl)-4-methyl-valeryl)-amino-3-N-(N-benzyl-4-piperidine-carbonyl)-amino-propan-2-one

Following the procedure of Example 34 (a), except substituting “N-benzyl-4-piperidine-carboxylic acid” for ““8-quinoline carboxylic acid”, the title compound was prepared: MS (ES+) 540.3 (M+H⁺).

### Example 40

Preparation of 1-N-(2-(3-biphenyl)-4-methyl-valeryl)-amino-3-N-(2-quinoline-carbonyl)-amino-propan-2-one

a) 1-N-(2-(3-biphenyl)-4methyl-valeryl)-amino-3-N-(2-quinoline-carbonyl)-amino-propan-2-one

Following the procedure of Example 35 (a), except substituting “2-quinoline-carboxylic acid” for ““8-quinoline carboxylic acid”, the title compound was prepared: MS (ES+) 494.2 (M+H⁺).

### Exampl 41

Preparation of 1-N-(2-(3-biphenyl)-4-methyl-valeryl)-amino-3-N-(2-carboxyl-phenyl-sulfonyl)-amino-propan-2-one

a) 1-N-(2-(3-biphenyl)-4-methyl-valeryl)-amino-3-N-(2-carboxyl-phenyl-sulfonyl)-amino-propan-2-one

1-N-(2-(3-biphenyl)-14methyl-valeryl)-amino-3-N-(2-carboxymethyl-phenyl-sulfonyl)-amino-propan-2-one (94 mg, 0.175 mmol) was dissolved in MeOH (10 ml), water (1 ml), then LiOH-H₂O (8 mg, 0.18 mmol) was added and the reaction was stirred for 15 minutes at RT. The reaction mixtrure was then quenched with 1N HCl in ether (0.2 ml), concentrated in vacuo, then chromatoagraphed on silca gel (60:40:1 EtOAc: hexanes: AcOH) to produce a white solid (60 mg, 66%): MS (ES+) 523.2 (M+H⁺), 555.2 (M+Ha⁺).

### Example 42

Preparation of 1-N-(2-(3-biphenyl)-4-methyl-valeryl)-amino-3-N-(4-C-tetrazole-phenyl-sulfonyl)-amino-propan-2-one

a) 1-N-(2-(3-biphenyl)-4-methyl-valeryl)-amino-3-N-(4-C-tetrazole-phenyl-sulfonyl)-amino-pmpan-2-one

1-N-(2-(3-biphenyl)-4-methyl-valeryl)-amino-3-N-(4-cyano-phenyl-sulfonyl)-amino-propan-2-one (300 mg, 0.6 mmol) was dissolved in N-methyl pyrolidinone (3 ml), then sodium azide (116 mg, 1.8 mmol) and triethyl amine-HCl (0.124 g, 0.9 mmol) was added and the reaction was heated to 100 degrees C. and was stirred for 5.5 h. The crude reaction mixture was cooled to RT, then chromatographed on silica gel (5% MeOH-1% AcOH-94% methylene chloride) to yield a whte solid (125 mg, 38%): MS (ES+) 547.2 (M+H⁺).

### Example 43

Preparation of 1-N-(2-(3-biphenyl)-4-methyl-valeryl)-amino-3-N-(3-(2-pvridyl-(phenyl acetyl)-amino-(S)-butan-2-one

a) Cbz-L-ala-bromo methyl ketone

Isobutyl chloroformate (2.74 ml, 21.2 mmol) was added dropwise to a solution of Cbz-L-alanine (4.7 g, 21.2 mmol) and N-methyl morpholine (2.32 ml, 21.2 mmol) in ThF (40 ml) at −40 degrees C. The reaction was stirred 15 min, then was filtered, and was washed with ether. Diazomethane from 12 g of 1-methyl-3-nitro-nitroso-guanidine and 36 ml of 40% KOH in ether (300 ml) was added and the reaction was placed in a refrigerator overnight (0 degrees C). 30% HBr/AcOH (14 ml) was added dropwise to the crude reaction mnixture and was stirred 5 minutes. The solution was washed with aqueous citric acid (50 ml×2), saturated aqueous sodium bicarbonate (3×150 ml), then brine (100 ml). The combined organics were dried with magnesium sulfate, filtered, and concentrated in vacuo to give a solid which was used in the next step without purification: MS (ES+) 360.3 (M+H⁺).

b) Cbz-ala-azido methyl ketone

Cbz-L-ala-bromo methyl ketone (1.5 g, 5 mmol) was dissolved in DMF (10 ml), then sodium azide (0.39 g, 6 mmol) and potassium fluoride (0.58 g, 7.5 mmol) was added and the reaction was stirred overnight. The reaction was partitioned between EtOAc and water, then the combined organic extracts were dried with magnesium sulfate, filtered, concentrated in vacuo, then chormatographed (2-5% MeOH, methylene chloride, silica gel) to provide the title compound as a white solid (0.5 g, 38%), IR (thin film): 2106.4 cm⁻¹.

c) (SN-Cbz-3-amino-1-azido-butan-2-ol

Cbz-ala-azido methyl ketone (0.5, 1.9 mmol) was dissolved in MeOH (10 ml) and sodium borohydride (0.144 g, 3.8 mmol) was added at 10 degrees C. and the reaction was stirred for 15 minutes. The reaction was quenched with water (10 ml) and was extracted with EtOAc (25 ml). The combined organic extracts were dried with magnesium sulfate, filtered, concentrated to give the title compound without further purification (0.5 g, quant.).

d) (S)-N-Cbz-3-amino-1-amino-butan-2-ol

(S)-N-Cbz-3-amino-1-azido-butan-2-ol (0.5 g, 1.9 mmol) was dissolved in MeOH (7.5 ml) and triethyl amine (1.0 ml, 7.1 mmol), propan-1,3-dithiol (1.07 ml, 10 mmol) was added and the reaction was stirred overnight, concentrated in vacuo, then the white solid was washed with hexane providing the title compound which was used in the next reaction without further purification: MS (ES+) 239.3 (M+H⁺).

e) 1-N-(Cbz)-amino-3-N-(3-(2-pyridyl-(phenyl acetyl)-amino-(S)-butan-2-ol

(S)-Cbz-3-amino-1-amino-butan-2-ol (0.452 g, 1.9 mmol), 3-(2-pyridyl)-phenyl acetic acid (0.4 g, 1.9 mmol) were dissolved in DMF (15 ml) and HOBT-H₂O (0.27 g, 2 mmol) EDCI (0.38 g, 2 mmol) and added, and the reaction was stirred overnight. The reaction was partitioned between EtOAc and 1 N NaOH, the combined organics were dried with magnesium sulfate, filtered, concentrated to give the title compound (0.33 g, 40%): MS (ES+) 434.2 (M+H⁺).

f) 1-N-(3-(2-pyridyl-(phenyl acetyl)-amino-(S)-butan-2-ol-3-amine

1-N-(Cbz)-amino-3-N-(3-(2-pyridyl-(phenyl acetyl)-amino-(S)-butan-2-ol (0.33 g, 0.76 mmol) was dissolved in EtOH (12 ml), then 10% Pd/C (0.08 g) was added and the reaction was stirred under a balloon of hydrogen gas overnight. The reaction was filtered through Celite, concentrated in vacuo, and was used in the next reaction without further purification: MS (ES+) 300.3 (M+H⁺).

g) 2-(3-biphenyl)-4methyl-valeryl chloride

Thionyl chloride (0.25 ml, 3.4 mmol) was added dropwise to a solution of 2-(3-biphenyl)-4-methyl-pentanoic acid (0.54 g, 2 mmol) in toluene (25 ml), then a drop of DMF was added, and the reaction mixture was stirred 2 h at RT. The reaction mixture was concentrated in vacuo and was used in the next reaction without further purification: IR (thin film): 1790.65 cm^{-1.}

h) 1-N-(2-(3-biphenyl)-4-methyl-valeryl)-amino-3-N-(3-(2-pyridyl-(phenyl acetyl)-amino-(S)-butan-2-ol

2-(3-biphenyl)-4-methyl-valeryl chloride (0.22 g, 0.76 mmol) was added dropwise to a solution of 1-N-(3-(2-pyridyl-(phenyl acetyl)-amino-(S)-butan-2-ol-3-amnine (0.28 g, 0.76 mmol), NMM (0.42 ml, 3.8 mmol) in DMF (10 ml) and the reaction was stirred 1 h. The reaction was extracted with EtOAc, 1N NaOH, and the combined organics were dried with MgSO₄, filtered, concentrated, and chromatographed (silica gel, 4% MeOH—CH₂Cl₂) to produce a white foam (0.24 g, 57%): MS (ES+) 550.3 (M+H⁺).

i) 1-N-(2-(3-biphenyl)-4-methyl-valeryl)-amino-3-N-(3-(2-pyridyl-(phenyl acetyl)-amino-(S)-butan-2-one

Following the procedure of Example 15 (c), except substituting “1-N-(2-(3-biphenyl)-4methyl-valeryl)-amino-3-N-(3-(2-pyridyl-(phenyl acetyl)-amino-(S)-butan-2-ol” for “1-N-(N-pentafluorobenzoyl-leucinyl)-amino-3-N-(3-(2-pyridyl)-phenyl acetyl)-amino-propan-2-ol”, the title compound was prepared: MS (ES+) 494.2 (M+H⁺).

### Example 44

Preparation of 1-N-(2-(3-biphenyl)-3-methyl-valeryl)-1-N-methyl-amino-3-N-(3-(2-pyridyl-(phenyl acetyl)-amino-propan-2-one

a) N-(2-(3-biphenyl)-3-methyl-valeryl)-1-N-methyl-glycine ethyl ester

2-(3-Biphenyl)-4-methyl-valeryl chloride (Example 44 (g), 2 g, 7 mmol) was added to a solution of sarcosine ethyl ester hydrochloride (1.07 g, 7 mmol) in NMM (1.9 ml, 17.5 mmol) in DMF (10 ml). The reaction was stirred at RT for 2.5 h, concentrated in vacuo, chromatographed (silica gel, 10% EtOAc/hexanes) to produce a clear liquid (2 g, 78%): MS (ES+) 368.4 (M+H⁺).

b) N-(2-(3-biphenyl)-3-methyl-valeryl)-1-N-methyl-glycine

LiOH-H2O (0.25 g, 6 mmol) was added to a solution of N-(2-(3-biphenyl)-3-methyl-valeryl)-1-N-methyl-glycine ethyl ester (2 g, 5.45 mmol) in THF (30 ml)/H₂O (3 ml) and was stirred for 2 h at RT. The reaction mixture was treated with 1N HCl in ether (7 ml), then was concentrated in vacuo to produce a white solid that was used in the next reaction without further purification: ¹H NMR (δ): 7.2-2.6 (m, 9H), 4.3 (d, 1H), 4.0 (d, 1H), 3.05 (s, 3H), 3.0 (s, rotamer), 0.8-1.0 (m, 6H).

c) 1-N-(2-(3-biphenyl)-3-methyl-valeryl)-1-N-methyl-amino-3-N-(3-(2-pyridyl-(phenyl acetyl)-amino-propan-2-ol

Following the procedure of Example 43 (a-e), except substituting “N-(2-(3-biphenyl)-3-methyl-valeryl)-1-N-methyl-glycine” for “Cbz-L-alanine”, the title compound was prepared: MS (ES+) 550.3 (M+H⁺).

d) 1-N-(2-(3-biphenyl)-3-methyl-valeryl)-1-N-methyl-amino-3-N-(3-(2-pyridyl-(phenyl acetyl)-amino-propan-2-one

Following the procedure of Example 15 (c), except substituting “1-N-(2-(3-biphenyl)-3-methyl-valeryl)-1-N-methyl-amino-3-N-(3-(2-pyridyl-(phenyl acetyl)-amino-propan-2-ol” for “1-N-(N-pentafluorobenzoyl-leucinyl)-amino-3-N-(3-(2-pyridyl)-phenyl acetyl)-amino-propan-2-ol”, the title compound was prepared: MS (ES+) 548.2 (M+H⁺).

### Example 45

Preparation of 1-N-(N-2-pyridyl carbonyl-leucinyl)-amino-3-N-(4-phenoxy-phenyl carbonyl)-amino-propan-2-one

a) 1-N-(N-2-pyridyl carbonyl-leucinyl)-amino-3-N-(4-phenoxy-phenyl carbonyl)-amino-propan-2-one

Following the procedure of Example 1 (a-c), except substituting “4-phenoxy-phenyl-carboxylic acid and EDCI” for “2-pyridine sulfonyl chloride”, and of Example 15 (c), except substituting “1-N-(N-2-pyridyl carbonyl-leucinyl)-amino-3-N-(4-phenoxy-phenyl carbonyl)-amino-propan-2-ol” for “1-N-(N-pentafluorobenzoyl-leucinyl)-amino-3-N-(3-(2-pyridyl)-phenyl acetyl)-amino-propan-2-ol”, the title compound was prepared: MS (ES+) 503.3 (M+H⁺).

### Example 46

Preparation of 1-N-(N-8-quinoline-carbonyl-leucinyl)-amino-3-N-(4-phenoxy-phenyl carbonyl)-amino-propan-2-one

a) 1-N-(N-8-quinoline-carbonyl-leucinyl)-amino-3-N-(4-phenoxy-phenyl carbonyl)-amino-propan-2-one

Following the procedure of Example 1 (a-c), except substituting “4-phenoxy-phenyl-carboxylic acid and EDCI” for “2-pyridine sulfonyl chloride” and “8-quinoline carboxylic acid” for “2-pyridine carboxylic acid”, and Example 15 (c), except substituting “1-N-(N-8-quinoline-carbonyl-leucinyl)-amino-3-N-(4-phenoxy-phenyl carbonyl)-amino-propan-2-ol” for “1-N-(N-pentafluorobenzoyl-leucinyl)-amino-3-N-(3-(2-pyridyl)-phenyl acetyl)-amino-propan-2-ol”, the title compound was prepared: MS (ES+) 553.3 (M+H⁺), 575.2 (M+Ha⁺).

### Example 47

Preparation of 1-N-(N-2-quinoline-carbonyl-leucinyl)-amino-3-N-(4-phenoxy-phenyl carbonyl)-amino-propan-2-one

a) 1-N-(N-2-quinoline-carbonyl-leucinyl)-amino-3-N-(4-phenoxy-phenyl carbonyl)-amino-propan-2-one

Following the procedure of Example 1 (a-c), except substituting “4-phenoxy-phenyl-carboxylic acid and EDCI” for “2-pyridine sulfonyl chloride” and “2-quinoline carboxylic acid” for “2-pyridine carboxylic acid”, and Example 15 (c), except substituting “1-N-(N-8-quinoline-carbonyl-leucinyl)-amino-3-N-(4-phenoxy-phenyl carbonyl)-amino-propan-2-ol” for “1-N-(N-pentafluorobenzoyl-leucinyl)-amino-3-N-(3-(2-pyridyl)-phenyl acetyl)-amino-propan-2-ol”, the title compound was prepared: MS (ES+) 553.2 (M+H⁺), 575.2 (M+Ha⁺).

### Example 48

Preparation of 1-N-(N-Cbz-norvalinyl)-amino-3-N-(8-quinoline-sulfonyl)-amino-propan-2-one

a) 1-N-(N-Cbz-norvalinyl)-amino-3-N-(8-quinoline-sulfonyl)-amino-propan-2-one

Following the procedure of Example 14 (d-e), except substituting “Cbz-norvaline” for “Cbz-leucine” and “8-quinoline sulfonyl chloride” for “3-(2-pyridyl)-phenyl acetic acid and EDCI”, the title compound was prepared: MS (ES+) 513.2 (M+H⁺).

### Example 49

Preparation of 1-N-(8-quinoline-sulfonyl)-amino-3-N-(8-quinoline-sulfonyl)-amino-propan-2-one

a) 1-N-(8-quinoline-sulfonyl)-amino-3-N-(8-quinoline-sulfonyl)-amino-propan-2-one

Following the procedure of Example 48, the title compound was prepared (side product): MS (ES+) 471.2 (M+H⁺).

### Example 50

Preparation of 1-N-(2-(3-biphenyl)-3-methyl-valeryl)-amino-3-N-(8-

uinoline-sulfonyl)-amino-propan-2-one

a) 1-N-(2-(3-biphenyl)-3-methyl-valeryl)-amino-3-N-(8-quinoline-sulfonyl)-amino-propan-2-one

Following the procedure of Example 31 (a-d), substituting “8-quinoline sulfonyl chloride” for “2-pyridyl-sulfonyl” and Example 15 (c), except substituting “1-N-(2-(3-biphenyl)-4methyl-pentamido)-amino-3-N-(8-quinoline-sulfonyl)-amino-propan-2-ol” for “1-N-(N-pentafluorobenzoyleucinyl)-amino-3-N-(3-(2-pyridyl)-phenyl acetyl)-amino-propan-2-ol ”, the title compound was prepared: MS (ES+) 530.3 (M+H⁺).

### Example 51

Preparation of 1-N-(2-(3-biphenyl)-3-methyl-valeryl)-amino-3-N-(2-(3-biphenyl)-3-methyl-valeryl)-amino-propan-2-one

a) 1-N-(2-(3-biphenyl)-3-methyl-valeryl)-amino-3-N-(2-(3-biphenyl)-3-methyl-valeryl)-amino-propan-2-one

Following the procedure of Example 50, the title compound was prepared (side product): MS (ES+) 611.3 (M+Ha⁺).

### Example 52

Preparation of 1-N-(N-(Cbz-norvalinyl)-amino-3-N-(N-(Cbz-norvalinyl)-amino-propan-2-one

a) 1-N-(N-(Cbz-norvalinyl)-amino-3-N-(N-(Cbz-norvalinyl)-amino-propan-2-one

Following the procedure of Example 48, the title compound was prepared (side product): MS (ES+) 577.3 (M+Ha⁺).

### Example 53

Preparation of 1-((3-biphenyl)-but-3-ene-1-carbonyl)-amino-3-N-(3-(2-pyridyl)-phenyl acetyl)-amino-propan-2-one

a) 2-(3-biphenyl)-pent-4-enoic acid

Following the procedure of Example 31 (d), except substituting “allyl bromide” for “isobutenyl bromide ”, the title compound was prepared: 1H NMR: d: 7.29-7.58 (m, 9H), 5.71-5.82 (m, 1H), 5.04 (d, 1H), 5.08 (d, 1H), 3.67 (t, 1H), 2.77-2.84 (m, 1H),2.46-2.56 (m, 1H).

b) 1-((3-biphenyl)-but-3-ene-1-carbonyl)-amino-3-N-(3-(2-pyridyl)-phenyl acetyl)-amino-propan-2-one

Following the procedure of Example 14 (a-d), except substituting “2-(3-biphenyl)-pent-4-enoic acid” for “Cbz-leucine” and Example 15 (c), except substituting “1-((3-biphenyl)-but-3-ene-1-carbonyl)-amino-3-N-(3-(2-pyridyl)-phenyl acetyl)-amino-propan-2-ol” for “1-N-(N-pentafluorobenzoyl-leucinyl)-amino-3-N-(3-(2-pyridyl)-phenyl acetyl)-amino-propan-2-ol”, the title compound was prepared: MS (ES+) 518.3 (M+H⁺), 540.3 (M+Ha⁺).

### Example 54

Preparation of 1-N-(2-(3-biphenyl)-but-3-ene-1-carbonyl)-amino-3-N-2-(3-biphenyl)-but-3-ene-1-carbon 1)-propan-2-one

a) 1-N- (2-(3-biphenyl)-but-3-ene-1-carbonyl)-amino-3-N-2-(3-biphenyl)-but-3-ene-1-carbonyl)-propan-2-one

Following the procedure of Example 53, the title compound was prepared (side product): MS (ES+) 557.3 (M+H⁺), 579.2 (M+Ha⁺).

### Example 55

Preparation of 1-(3-biphenyl)-ethyl-cyclopropane-1-carbonyl)-amino-3-N-(3-(2-pvridyl)-phenyl acetyl)-amino-propan-2-one

a) 2-(3-biphenyl)-3-cyclopropyl-propanoic acid

Following the procedure of Example 29 (a-b), except substituting “2-(3-biphenyl)-pentenoic acid” for “Cbz-L-α-allyl-glycine”, the title compound was prepared: 1H NMR: d: 7.33-7.73 (m, 9H), 3.77 (t, 1H), 1,93-2.01 (m, 1H), 1.78-1.85 (m, 1H), 0.66-0.71 (m, 1H), 0.41-0.48 (m, 2H), 0.05-0.17 (m, 2H).

b) 1-(3-biphenyl)-ethyl-cyclopropane-1-carbonyl)-amino-3-N-(3-(2-pyridyl)-phenyl acetyl)-amino-propan-2-one

Following the procedure of Example 14 (a-d), except substituting “2-(3-biphenyl)-3-cyclopropyl-propanoic acid” for “Cbz-leucine” and Example 15 (c), except substituting “1-(3-biphenyl)-ethyl-cyclopropane-1-carbonyl)-amino-3-N-(3-(2-pyridyl)-phenyl acetyl)-amino-propan-2-ol” for “1-N-(N-pentafluorobenzoyl-leucinyl)-amino-3-N-(3-(2-pyridyl)-phenyl acetyl)-amino-propan-2-ol”, the title compound was prepared: MS (ES+) 532.2 (M+H⁺).

### Example 56

Preparation of 1-N-(2-(3-biphenyl)-3-methyl-but-3-ene-1-carbonyl)-amino-3-N-(3-(2-pyridyl)-phenyl acetyl)-amino-propan-2-one

a) 1-N-(2-(3-biphenyl)-3-methyl-but-3-ene-1-carbonyl)-amino-3-N-(3-(2-pyridyl)-phenyl acetyl)-amino-propan-2-one

Following the procedure of Example 14 (a-d), except substituting “2-(3-biphenyl)-4-methyl-pent-4-enoic acid (Example 31 (d)” for “Cbz-leucine” and Example 15 (c), except substituting “1-(3-biphenyl)-3-methyl-but-3-ene-1-carbonyl)-amino-3-N-(3-(2-pyridyl)-phenyl acetyl)-amino-propan-2-ol” for “1-N-(N-pentafluorobenzoyl-leucinyl)-amino-3-N-(3-(2-pyridyl)-phenyl acetyl)-amino-propan-2-ol”, the title compound was prepared: MS (ES+) 532.2 (M+H⁺), 554.2 (M+Ha⁺).

### Example 57

Preparation of 1-(3-biphenyl)-3-methyl-but-3-ene-1-carbonyl)-amino)-3-(3-biphenyl)-3-methyl-but-3-ene-1-carbonyl)-amino-propan-2-one

a) 1-(3-biphenyl)-3-methyl-but-3-ene-1-carbonyl)-amino)-3-(3-biphenyl)-3-methyl-but-3-ene-1-caibonyl)-amino-propan-2-one

Following the procedure of Example 56, the title compound was prepared (side product): MS (ES+) 585.3 (M+H⁺), 607.3 (M+Ha⁺).

### Example 58

Preparation of 1-N-(N-(trans-4-propyl cyclohexyl-carbonyl)-leucinyl)-amino-3-N-(3-(2-pyridyl)-phenyl acetyl)-amino-propan-2-one

a) Preparation of 1-N-(N-(trans-4-propyl cyclohexyl-carbonyl)-leucinyl)-amino-3-N-(3-(2-pyridyl)-phenyl acetyl)-amino-propan-2-one

Following the procedure of Example 15 (a-c), except substituting “trans-4-propyl-cyclohexyl carboxylic acid” for “pentafluorobenzoic acid”, the title compound was prepared: MS (ES⁺) 549.3 (M+H⁺).

### Example 59

Preparation of 1-N-(N-(2-quinoxaline-carbonyl)-leucinyl)-amino-3-N-(3-(2-pyridyl)-phenyl acetyl)-amino-2-propan-2-one

a) Preparation of 1-N-(N-(2-quinoxaline-carbonyl)-leucinyl)-amino-3-N-(3-(2-pyridyl)-phenyl acetyl)-amnino-propan-2-one

Following the procedure of Example 15 (a-c), except substituting “2-quinoxaline-carboxylic acid” for “pentafluorobenzoic acid”, the title compound was prepared: MS (ES⁺) 553.1 (M+H⁺).

### Example 60

Preparation of 1-N-(N-(5-(2.3-dihydro-benzofuran)-carbonyl)-leucinyl)-amino-3-N-(3-(2-pyridyl)-phenyl acetyl)-amino-propan-2-one

a) 1-N-(N-(2-(2,3-dihydro-benzofuran)-carbonyl)-leucinyl)-amino-3-N-(3-(2-pyridyl)-phenyl acetyl)-amino-propan-2-one

Following the procedure of Example 15 (a-c), except substituting “5-(2,3-dihydro-benzofuran)-carboxylic acid” for “pentafluorobenzoic acid”, the title compound was prepared: MS (ES⁺) 543.2 (M+H⁺).

### Example 61

Preparation of 1-N-(N-(N-methyl-2-indole-carbonyl)-leucinyl)-amino-3-N-(3-(2-2pyridyl)-phenyl acetyl)-amino-propan-2-one

a) Preparation of 1-N-(N-(N -methyl-2-indole-carbonyl)-leucinyl)-amino-3-N-(3-(2-pyridyl)-phenyl acetyl)-amino-propan-2-one

Following the procedure of Example 15 (a-c), except substituting “N-methyl-2-indole-carboxylic acid” for “pentafluorobenzoic acid”, the title compound was prepared: MS (ES⁺) 554.1 (M+H⁺).

### Example 62

Preparation of 1-N-(N-(cyclohexyl-carbonyl)-leucinyl)-amino-3-N-(3-(2-pyridyl)-phenyl acetyl)-amino-propan-2-one

a) Preparation of 1-N-(N-(cyclohexyl-carbonyl)-leucinyl)-amino-3-N-(3-(2-pyridyl)-phenyl acetyl)-amino-propan-2-one

Following the procedure of Example 15 (a-c), except substituting “cyclohexyl carboxylic acid” for “pentafluorobenzoic acid”, the title compound was prepared: MS (ES⁺) 507.4 (M+H⁺).

### Example 63

Preparation of 1-N-(N-(2-chloro-benzoyl)-leucinyl)-amino-3-N-(3-(2-pyridyl)-phenyl acetyl)-amino-propan-2-one

a) 1-N-(N-(2-chloro-benzoyl)-leucinyl)-amino-3-N-(3-(2-pyridyl)-phenyl acetyl)-amino-propan-2-one

Following the procedure of Example 15 (a-c), except substituting “2-chloro-benzoic acid” for “pentafluorobenzoic acid”, the title compound was prepared: MS (ES⁺) 535.2 5v (M+H⁺)

### Example 64

Preparation of 1-N-(N-(2-benzofuran-carbonyl)-leucinyl)-amino-3-N-(3-(2-pvridyl)-phenyl acetyl)-amino-propan-2-one

a) 1-N-(N-(2-benzofuran-carbonyl)-leucinyl)-amino-3-N-(3-(2-pyridyl)-phenyl acetyl)-amino-propan-2-one

Following the procedure of Example 15 (a-c), except substituting “2-benzofuran-carboxylic acid” for “pentafluorobenzoic acid”, the title compound was prepared: MS (ES⁺) 541.2 (M+H⁺), 573.3 (M+Ha⁺).

### Example 65

Preparation of 1-N-(N-(3-phenoxy-phenyl-carbonyl)-leucinyl)-amino-3-N-(3-(2-pyridyl)-phenyl acetyl)-amino-propan-2-one

a) 1-N-(N-(3-phenoxy-phenyl-carbonyl)-leucinyl)-amino-3-N-(3-(2-pyridyl)-phenyl acetyl)-amino-propan-2-one

Following the procedure of Example 15 (a-c), except substituting “3-phenoxy-phenyl carboxylic acid” for “pentafluorobenzoic acid”, the title compound was prepared: MS (ES⁺) 593.2 (M+H⁺).

### Example 66

Preparation of 1-N-(N-(4-phenoxy-phenyl-carbonyl)-leucinyl)-amino-3-N-(3-(2-pyridyl)-phenyl acetyl)-amino-propan-2-one

a) 1-N-(N-(4phenoxy-phenyl-carbonyl)-leucinyl)-amino-3-N-(3-(2-pyridyl)-phenyl acetyl)-amino-propan-2-one

Following the procedure of Example 15 (a-c), except substituting “4-phenoxy-phenyl carboxylic acid” for “pentafluorobenzoic acid”, the title compound was prepared: MS (ES⁺) 593.2 (M+H⁺).

### Example 67

Preparation of 1-N-(N-(3-methoxy-2-quinoline-carbonyl)-leucinyl)-amino-3-N-(3-(2-pyridyl)-phenyl acetyl)-amino-propan-2-one

a) 1-N-(N-(3-methoxy-2-quinoline-carbonyl)-leucinyl)-amino-3-N-(3-(2-pyridyl)-phenyl acetyl)-amino-propan-2-one

Following the procedure of Example 15 (a-c), except substituting “3-methoxy-2-quinoline-carboxylic acid” for “pentafluorobenzoic acid”, the title compound was prepared: MS (ES⁺) 581.2 (M+H⁺).

### Example 68

Preparation of 1-N-(N-Cbz-leucinyl)-amino-3-N-(3-(2-pyridyl-(phenyl acetyl)-amino-(S)-butan-2-one

a) Preparation of 1-N-(N-Cbz-leucinyl)-amino-3-N-(3-(2-pyridyl-(phenyl acetyl)-amino-(S)-butan-2-one

Following the procedure of Example 44 (a-i), except substituting “Cbz-leucine and HBTU” for “2-(3-biphenyl)-4-methyl-pentanoic acid and thionyl chloride”, the title compound was prepared: MS (ES⁺) 545.3 (M+H⁺).

### Example 69

Preparation of 1-N-(N-(4-fluorobenzoyl)-leucinyl)-amino-3-N-(3-(2-pyridyl-(phenyl acetyl)-amino-(S)-butan-2-one

a) 1-N-(N-Boc-leucinyl)-amino-3-N-(3-(2-pyridyl-(phenyl acetyl)-amino-(S)-butan-2-ol

Following the procedure of Example 44 (a-i), except substituting “Boc-leucine and HBTU” for “2-(3-biphenyl)-methyl-pentanoic acid and thionyl chloride”, the title compound was prepared: MS (ES⁺) 513.2 (M+H⁺).

b) 1-N-(leucinyl)-amino-3-N-(3-(2-pyridyl-(phenyl acetyl)-amino-(S)-butan-2-ol

Following the procedure of Example 1 (b), except substituting “1-N-(N-Boc-leucinyl)-amino-3-N-(3-(2-pyridyl-(phenyl acetyl)-amino-(S)-butan-2-ol” for “1-N-(Boc-leucinyl)-amino-3-N-(2-pyridyl-sulfonyl)-amino-propan-2-ol”, the title compound was prepared: MS (ES⁺) 413.1 (M+H⁺).

c) 1-N-(N-(4-fluorobenzoyl)-leucinyl)-amino-3-N-(3-(2-pyridyl-(phenyl acetyl)-amnino-(S)-butan-2-one

Following the procedure of Example 15 (b-c), except substituting “1-N-(leucinyl)-amino-3-N-(3-(2-pyridyl-(phenyl acetyl)-amino-(S)-butan-2-ol “for” leucinyl-amino-3-N-(3-(2-pyridyl)-phenyl acetyl)-amino-propan-2-ol” and “4-fluorobenzoic acid” for “pentafluorobenzoic acid”, the title compound was prepared: MS (ES⁺) 533.3 (M+H⁺), 555.1 (M+Ha⁺).

### Example 70

Preparation of 1-N-(N-(2-benzothiophene-carbonyl)-leucinyl)-amino-3-N-(3-(2-pyridyl-(phenyl acetyl)-amino-(S)-butan-2-one

a) 1-N-(N-(2-benzothiophene-carbonyl)-leucinyl)-amino-3-N-(3-(2-pyridyl-(phenyl acetyl)-amino-(S)-butan-2-one

Following the procedure of Example 79 (a-c), except substituting “2-benzothiophene carboxylic acid” for “4-fluorobenzoic acid”, the title compound was prepared: MS (ES⁺) 571.2 (M+H⁺).

### Example 71

Preparation of 1-N-(N-(2-pyridyl-methyleneoxy-carbonyl)-leucinyl)-amino-3-N-(1-naphthalene sulfonyl)-amino-propan-2-one

a) 1-N-(N-(2-pyridyl-methyleneoxy-carbonyl)-leucinyl)-amino-3-N-(1-naphthalene sulfonyl)-amino-propan-2-one

Following the procedure of Example 14 (d-e), except substituting “2-pyridyl methyleneoxy carbonyl-leucine” for “Cbz-leucine” and “1-naphthalene sulfonyl chloride” for “3-(2-pyridyl)-phenyl acetic acid and EDCI”, the title compound was prepared: MS (ES+) 527.2 (M+H⁺).

### Example 72

Preparation of 1-N-(N-(2-pyridyl-methyleneoxy-carbonyl)-leucinyl)-amino-3-N-(1,3-dimethyl-5-chloro-pyrazole-4-sulfonyl)-amino-propan-2-one

a) 1-N-(N-(2-pyridyl-methyleneoxy-carbonyl)-leucinyl)-amino-3-N-(1,3-dimethyl-5-chloro-pyrazole-4-sulfonyl)-amino-propan-2-one

Following the procedure of Example 14 (d-e), except substituting “2-pyridyl methyleneoxy carbonyl-leucine” for “Cbz-leucine” and “1,3-dimethyl-5-chloro-pyrazole-4-sulfonyl chloride” for “3-(2-pyridyl)-phenyl acetic acid and EDCI”, the title compound was prepared: MS (ES+)530.2 (M+H⁺).

### Example 73

Preparation of 1-N-(N-(2-pyridyl-methyleneoxy-carbonyl)-leucinyl)-amino-3-N-(benzo-2,1,3-thiadiazole-4sulfonyl)-amino-2-propanone)

a) 1-N-(N-(2-pyridyl-methyleneoxy-carbonyl)-leucinyl)-amino-3-N-(benzo-2,1,3-thiadiazole-4-sulfonyl)-amino-2-propanone)

Following the procedure of Example 14 (d-e), except substituting “2-pyridyl methyleneoxy carbonyl-leucine” for “Cbz-leucine” and “benzo-2,1,3-thiadiazole-4-sulfonyl chloride” for “3-(2-pyridyl)-phenyl acetic acid and EDCI”, the title compound was prepared: MS (ES+) 535.2 (M+H⁺).

### Example 74

Preparation of 1-N-(N-(2-pyridyl-methyleneoxy-carbonyl)-leucinyl)-amino-3-N-(3.5-dimethyl-isoxazole-4-sulfonyl)-amino-propan-2-one

a) 1-N-(N-(2-pyridyi-methyleneoxy-carbonyl)-leucinyl)-amino-3-N-(3,5-dimethyl-isoxazole-4sulfonyl)-amino-propan-2-one

Following the procedure of Example 14 (d-e), except substituting “2-pyridyl methyleneoxy carbonyl-4eucine” for “Cbz-leucine” and “3,5-dimethyl-isoxazole-4sulfonyl chloride” for “3-(2-pyridyl)-phenyl acetic acid and EDCI”, the title compound was prepared: MS (ES+) 496.2 (M+H⁺).

### Example 75

Preparation of 1-N-(N-(4-trifluoromethyl benzoyl)-leucinyl)-amino-3-N-(3-(2-pyridyl)-phenyl acetyl)-amino-propan-2-one

a) 1-N-(N-(4-trifluoromethyl benzoyl)-leucinyl)-amino-3-N-(3-(2-pyridyl)-phenyl acetyl)-amino-propan-2-one

Following the procedure of Example 15 (a-c), except substituting “4phenoxy-phenyl carboxylic acid” for “pentafluorobenzoic acid”, the title compound was prepared: MS (ES⁺) 569.1 (M+H⁺).

### Example 76

Preparation of 1-N-(N-(6-benzthiazole-carbonyl)-leucinyl)-amino-3-N-(3-(2-pyridyl)-phenyl acetyl)-amino-propan-2-one

a) 1-N-(N-(6-benzthiazole-carbonyl)-leucinyl)-amino-3-N-(3-(2-pyridyl)-phenyl acetyl)-amino-propan-2-one

Following the procedure of Example 15 (a-c), except substituting “6-benzthiazole carboxylic acid” for “pentafluorobenzoic acid”, the title compound was prepared: MS (ES⁺) 558.2 (M+H⁺).

### Example 77

Preparation of 1-N-(N-(6-quinoline-carbonyl)-leucinyl)-amino-3-N-(3-(pyridyl)-phenyl acetyl)-amino-propan-2-one

a) 1-N-(N-(6-quinoline-carbonyl)-leucinyl)-amino-3-N-(3-(2-pyridyl)-phenyl acetyl)-amino-propan-2-one

Following the procedure of Example 15 (a-c), except substituting “6-quinoline carboxylic acid” for “pentafluorobenzoic acid”, the title compound was prepared: MS (ES⁺) 552.3 (M+H⁺).

### Example 78

Preparation of 1-N-(N-(4-fluoro-benzoyl)-norleucinyl)-amino-3-N-(3-(2-pyridyl)-phenyl acetyl-amino-propan-2-one

a) 1-N-(N-(4-fluoro-benzoyl)-norleucinyl)-amino-3-N-(3-(2-pyridyl)-phenyl acetyl)-amino-propan-2-one

Following the procedure of Example 15 (a-c), except substituting “1-N-(N-Cbz-norleucinyl)-amino-3-N-(3-(2-pyridyl)-phenyl acetyl)-amino-propan-2-ol” (cf. Example 27) for “1-N-(N-Cbz-leucinyl)-amino-3-N-(3-(2-pyridyl)-phenyl acetyl)-amino-propan-2-ol” and “4-fluorobenzoic acid” for “pentafluorobenzoic acid”, the title compound was prepared: MS (ES⁺) 519.2 (M+H⁺).

### Example 79

Preparation of 1-N-(N-(2-naphthyl-carbonyl)-norleucinyl)-amino-3-N-(3-(2-pyridyl)-phenyl acetyl)-amino-propan-2-one

a) 1-N-(N-(2-naphthyl-carbonyl)-norleucinyl)-amino-3-N-(3-(2-pyridyl)-phenyl acetyl)-amino-propan-2-one

Following the procedure of Example 78, except substituting “2-naphthyl carboxylic acid” for “4-fluorobenzoic acid”, the title compound was prepared: MS (ES⁺) 551.2 (M+H⁺).

### Example 80

Preparation of 1-N-(N-(3,4-dimethoxy-benzoyl)-norleucinyl)-amino-3-N-(3-(2-pyridyl)-phenyl acetyl)-amino-propan-2-one

a) 1-N-(N-(3,4-dimethoxy-benzoyl)-norleucinyl)-amino-3-N-(3-(2-pyridyl)-phenyl acetyl-amino-propan-2-one

Following the procedure of Example 78, except substituting “3,4-dimethoxybenzoic acid” for “4-fluorobenzoic acid”, the title compound was prepared: MS (ES⁺) 561.2 (M+H⁺), 1121.3 (2M+H⁺)

### Example 81

Preparation of 1-N-(N-(5-benzothiophene-carbonyl)-norleucinyl)-amino-3-N-(3-(2-pyridyl)-phenyl acetyl)-amino-propan-2-one

a) 1-N-(N-(5-benzothiophene-carbonyl)-norleucinyl)-amino-3-N-(3-(2-pyridyl)-phenyl acetyl)-amino-propan-2-one

Following the procedure of Example 78, except substituting “5-thiophene-carboxylic acid” for “4-fluorobenzoic acid”, the title compound is prepared.

### Example 82

Preparation of 3-N-(N-Cbz-leucinyl)-amino-1-N-(phenyl)-5-methyl-hexan-2-one

a) Cbz-leu-leu-bromo methyl ketone

Isobutyl chloroformate (1.37 ml, 10.58 mmol) was added dropwise to a solution of Cbz-leu-leu-OH (4.0 g, 10.58 mmol) and N-methyl morpholine (1.16 ml, 10.58 mmol) in THF (20 ml) at −40 degrees C. The reaction was stirred 15 min, then was filtered, and was washed with ether. Diazomethane (mmol from 5.9 g of 1-methyl-3-nitro-nitroso-guanidine and 18 ml of 40% KOH in 150 ml of ether) in ether (50 ml) was added and the reaction was placed in a refrigerator overnight. 30% HBr/AcOH (7.0 ml) was added dropwise to the crude reaction mixture and was stirred 5 minutes. The solution was washed with 15% aqueous citric acid, saturated aqueous sodium bicarbonate, then brine. The combined organics were dried with magnesium sulfate, filtered, and concentrated in vacuo to give a solid which was used in the next step without purification: MS (ES⁺) 455.4, 457.4 (M+H⁺), 477.3,479.3 (M+H⁺).

b) a) (S)-3-N-(N-Cbz-leucinyl)-amino-1-N-(phenyl)-5-methyl-bexan-2-one

Cbz-Leu-LeuCH₂Br (0.1 g, 0.22 mmol) was dissolved in DMF (1.0 ml), then potassium fluoride (0.02 g, 0.33 mmol) and aniline (0.061 g, 0.66 mmol) were added and the reaction mixture was stirred at RT overnight. The reaction was extracted with EtOAc/H₂O, the combined organic extracts were dried with magnesium sulfate, filtered, concentrated in vacuo and chromatographed to provide the title compound as a white solid (18 mg, 18%): MS (ES⁺) 468A (M+H⁺).

The above specification and Examples fully disclose how to make and use the compounds of the present invention. However, the present invention is not limited to the particular embodiments described hereinabove, but includes all modifications thereof within the scope of the following claims. The various references to journals, patents and other publications which are cited herein comprise the state of the art and are incorporated herein by reference as though fully set forth.

We claim:

1. A compound of Formula I: 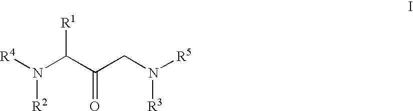
wherein:
R¹, R² and R³ are independently selected from the group consisting of H, C₁₋₆ alkyl, C₃₋₁₁cycloalkyl, C₂₋₆ alkenyl, C₂₋₆ alkynyl, Ar, Het, C₁₋₆ alkyl-Ar, C₃₋₁₁cycloalkyl-Ar, C₂₋₆ alkenyl-Ar, C₂₋₆ alkynyl-Ar; C₁₋₆ alkyl-Het, C₃₋₁₁cycloalkyl-Het, C₂₋₆ alkenyl-Het, and C₂₋₆ alkynyl-Het;
R⁴ is selected from the group consisting of N—(R⁶)—NHCH(C₁₋₆ alkyl)—CO, N,N—R⁶—(C₁₋₆ alkyl)-N(C₁₋₆ alkyl)-CO, N—(R⁶)—NHCH(C₂₋₆ alkenyl)-CO—, N—(R⁶)—NHCH(C₂₋₆alkynyl)-CO—, N—(R⁶)—NHCH(C₁₋₆ alkyl-Ar)—CO—, N—(R⁶)—NHCH(C₂₋₆ alkenylAr)—CO—, N—(R⁶)—NHCH(C₂₋₆ alkynyl-Ar)—CO—, N—(R⁶)—NHCH(C₁₋₆ alkyl-Het)-CO—, N—(R⁶)—NHCH(C₂₋₆ alkenyl-Het)-CO—, N—(R⁶)—NHCH(C₂₋₆ alkynyl-Het)-CO—, ArCO, Ar—C₁₋₆ alkyl-CO, Ar—SO₂, Ar—C₁₋₆ alkyl-SO₂, Het-CO, Het-C₁₋₆ alkyl-CO, Het-SO₂, and Het-C₁₋₆ alkyl-SO₂;
R⁵ is selected from the group consisting of N—R⁷-amino acid, C₁₋₆ alkyl CO, C₃₋₁₁cycloalkyl-CO, ArCO, Ar—C₁₋₆ alkyl-CO, Ar—SO₂, Ar—C₁₋₆ alkyl-SO₂, Het-CO, Het-C₁₋₆alkyl-CO, Het-SO₂, C₁₋₆ alkyl; Ar—CO₆ alkyl-; Het-CO6 alkyl-;
R⁶ and R⁷ are independently selected from the group consisting of Ar—(C₁₋₆ alkyl)-O—CO, Het-(C₁₋₆ alkyl)-O—CO, Ar—CO, Ar—SO₂, Het-CO, Het-SO₂, C₁₋₆ alkyl-CO, C₃₋₁₁cycloalkyl-CO, C₁₋₆ alkyl-SO₂, C₂₋₆ alkenyl-CO,C₂₋₆ alkenyl-SO₂, C₂₋₆ alkynyl-CO; C₂₋₆ alkynyl-SO₂, ArC₁₋₆ alkyl-CO, ArC₁₋₆ alkyl-SO₂, ArC₂₋₆ alkenyl-CO, ArC₂₋₆alkenyl-SO₂, Ar—C₂₋₆ alkynyl-CO,Ar—C₂₋₆ alkynyl-SO₂, Het-C₁₋₆ alkyl-CO, Het-C₁₋₆ alkyl-SO₂, Het-C₂₋₆ alkenyl-CO, Het-C₂₋₆ alkenyl-SO₂, Het-C₂₋₆ alkynyl-CO, and Het-C₂₋₆ alkynyl-SO₂;
and pharmaceutically acceptable salts, hydrates and solvates thereof.

2. A compound according to claim 1 wherein R¹, R² and R³ are independently selected from the group consisting of methyl, isobutyl, phenyl, benzyl, and isonicotinyl.

3. A compound according to claim 1 wherein R¹, R² and R³ are H.

4. A compound according to claim 1 wherein R⁴ is selected from the group consisting of N—R⁶-leucinyl, N—R⁶-norleucinyl-, N—R⁶-norvalinyl-, N—R⁶-isoleucinyl-, N—R⁶-α-allyl-glycinyl-, N—R⁶-α-(cyclopropylmethyl)-glycinyl-, N—R⁶-β-tert-butyl-alaninyl-2-, N—R⁶-homo-leucinyl-, N,N—R⁶-methyl-leucinyl-, 3-phenoxy-benzoyl, 4-phenoxy-benzoyl-, or 2-benzyloxy-benzoyl, 4biphenyl acetyl-, 2-(4-biphenyl)-4methyl-valeryl, 2-(3-biphenyl)-4methyl-valeryl, 1-(3-biphenyl)-but-3-ene-1-carbonyl, 1-(3-biphenyl)-ethyl-cyclopropane-1-carbonyl, 1-(3-biphenyl)-3-methyl-but-3-ene-1-carbonyl, 3-(2-pyridyl)-phenyl acetyl, 3-(3-pyridyl)-phenyl acetyl, 3-phenoxy-phenyl sulfonyl, 4-phenoxy-phenyl sulfonyl, 3-(4-(3-chloro-2-cyano-phenoxy)-phenyl sulfonyl, and 8-quinoline sulfonyl-.

5. A compound according to claim 1 wherein N—R⁷-amino acid is selected from the group consisting of N—(R⁷)—NHCH(C₁₋₆ alkyl)-CO, N—(R⁷)—NHCH(C₂₋₆ alkenyl)-CO—, N—(R⁷)—NHCH(C₂₋₆ alkynyl)-CO—, N—(R⁷)—NHCH(C₁₋₆ alkyl-Ar)—CO—, N—(R⁷)—NHCH(C₂₋₆ alkenylAr)—CO—, N—(R⁷)—NHCH(C₂₋₆ alkynyl-Ar)—CO—, R⁷-γ-t-butyl-glutamyl-, R⁷-glutamyl-, and N,N—R⁷—(C₁-C₆ alkyl)-leucinyl-.

6. A compound according to claim 1 wherein R⁵ is selected from the group consisting of N—R⁷-leucinyl-, N—R⁷-norleucinyl-, N—R⁷-norvalinyl-,N—R⁷-isoleucinyl-, N—R⁷-α-allyl-glycinyl-, N—R⁷-α-(cyclopropylmethyl)-glycinyl-, N—R⁷-β-tert-butyl-alaninyl-, N—R⁷-homo-leucinyl, N—(R⁷)-phenylalaninyl, acetyl, benzoyl, 3-phenoxy-benzoyl, 4-phenoxy-benzoyl, 2-benzyloxy benzoyl, 3-benzyloxy benzoyl, or 4-benzyloxy benzoyl, 2-(4-biphenyl)-4-methyl-valeryl, 2-(3-biphenyl)-4-methyl-valeryl, 1-(3-biphenyl)-but-3-ene-1-carbonyl, 1-(3-biphenyl)-ethyl-2-cyclopropane-1-carbonyl, 1-(3-biphenyl)-3-methyl-but-3-ene-1-carbonyl, 3-(2-pyridyl)-phenyl acetyl, 3-(3-pyridyl)-phenyl acetyl, 4-biphenyl acetyl-, 3-biphenyl acetyl-, 8-quinoline sulfonyl-, 3-biphenyl sulfonyl-, 4-cyano-phenyl sulfonyl, 2-carboxyl-phenyl sulfonyl, 2-carboxymethyl-phenyl sulfonyl-, 4-C-tetrazole-phenyl sulfonyl, 1-naphthalene sulfonyl, 3-phenoxy-phenyl sulfonyl, 4-phenoxy-phenyl sulfonyl, 3-(4-(3-chloro-2-cyano-phenoxy)-phenyl sulfonyl-, 4-biphenyl sulfonyl-, 2-dibenzofuran-sulfonyl, 8-quinoline carbonyl-, 6-quinoline carbonyl-, 2-pyridine carbonyl, 5-(2-pyridyl)-thiophene carbonyl, N-benzyl-4piperidinyl carbonyl, 2-quinoline carbonyl-, 2-pyridyl sulfonyl, 1,3-dimnethyl-5-chloro-pyrazole-4-sulfonyl, 3,5-dimethyl-isoxazole-4-sulfonyl, benzo-2,1,3-thiadiazole-4- sulfonyl, phenyl-sulfone-5-thiophene-2-sulfonyl-, 2-carboxymethyl thiophene-sulfonyl, 2,5-dichlorothiophene-3-sulfonyl-, and phenyl.

7. A compound according to claim 1 wherein R⁶ and R⁷ are independently selected from the group consisting of benzyloxycarbonyl, 2-pyridyl methyloxycarbonyl, 3-pyridyl methyloxycarbonyl, 4-pyridyl methyloxycarbonyl, benzoyl-, 1-naphthoyl-, 2-naphthoyl-, 4-phenoxy-benzoyl-, 3-phenoxy-benzoyl-, 2-phenoxy-benzoyl-, 2-chloro-benzoyl-, 4-fluoro-benzoyl, 3,4-difluoro benzoyl-, 4-trifluoromethyl benzoyl-, 2-chlorobenzoyl-, 4-carboxymethyl-benzoyl-, 4-carboxyl-benzoyl-, N,N-dimethyl glycinyl-, 2-pyridyl carbonyl-, 3-pyridyl carbonyl, 4-pyridyl carbonyl-, 2-quinoline carbonyl-, 3-quinoline carbonyl-, 4-quinoline carbonyl-, 5-quinoline carbonyl-, 6-quinoline carbonyl-, 7-quinoline carbonyl-, 8-quinoline carbonyl-, 1-isoquinoline caronyl-, 3- isoquinoline carbonyl-, isoquinoline carbonyl-, 5- isoquinoline carbonyl-, 6- isoquinoline carbonyl-, 7- isoquinoline carbony-, 8-isoquinoline carbonyl-, 1-benzothiophene carbonyl-, 1-benzofurancarbonyl-, 5-indole-carbonyl-sulfonyl-, N-methyl-prolinyl-, 2-quinoxaline-carbonyl-, 5-(2,3-dihydrobenzofuran-carbonyl-, 2-benzofuran-carbonyl-, 2-benzothiophene-carbonyl-, N-morpholino-carbonyl-, N-methyl-piperidine-carbonyl-, N-pyrazole-carbonyl-, 2-pyridyl sulfonyl-, 3-pyridyl sulfonyl, 4-pyridyl sulfonyl, 2-quinoline sulfonyl-, 3-quinoline sulfonyl-, 4-quinoline sulfonyl-, 5-quinoline sulfonyl-, 6-quinoline sulfonyl-, 7-quinoline sulfonyl-, 8-quinoline sulfonyl-, 1- isoquinoline sulfonyl-, 3- isoquinoline sulfonyl-, 4- isoquinoline sulfonyl-, 5-isoquinoline sulfonyl-, 6- isoquinoline sulfonyl-, 7- isoquinoline sulfonyl-, 8- isoquinoline sulfonyl-, acetyl, trans-4-propyl-cyclohexyl-carbonyl-, cyclohexyl-carbonyl-, 4-imidazole acetyl-, 2-pyridyl acetyl, 3-pyridyl acetyl, 4pyridyl acetyl-, and N-morpholine acetyl.

8. A compound according to claim 1 wherein:
R¹ is H or C₁₋₆ alkyl;
R² and R³ are H;
R⁴ is N—(R⁶)—NHCH(C₁₋₆ alkyl)-CO, N,N—R⁶—(C₁₋₆ alkyl)—N(C₁₋₆ alkyl)-CO, or Ar—C₁₋₆ alkyl-CO;
R⁵ is N—R⁷-norvalinyl-, Ar—C₁₋₆ alkyl-CO, Het-SO₂, Het-CO, ArCO, Ar—SO₂, or Ar—.

9. A compound according to claim 8 wherein R⁴ is N—R⁶-leucinyl, N—R⁶-norleucinyl, N—R⁶-norvalinyl, N—R⁶-isoleucinyl, N—R⁶-α-allyl-glycinyl, N—R⁶-α-(cyclopropylmethyl)-glycinyl-, or N—R⁶- L-β-tert-butyl-alaninyl.

10. A compound according to claim 8 wherein N,N—R⁶-(C₁₋₆ alkyl)-N(C₁₋₆ alkyl)-CO is N,N—R⁶-methyl-leucinyl-.

11. A compound according to claim 8 wherein:
R¹ is H or Me;
R⁴ is selected from the group consisting of N-(2-pyridyl carbonyl)-leucinyl, N-(8-quinoline carbonyl)-leucinyl, N-(6- quinoline carbonyl)-leucinyl, N-(2-quinoline carbonyl)-leucinyl, N-(4-imidazole acetyl)-leucinyl, N-benzoyl-leucinyl, N-(2-pyridyl sulfonyl)-leucinyl, N-(1-isoquinoline carbonyl)-leucinyl, N-(N-morpholine acetyl)-leucinyl, N-(N-methyl prolinyl)-leucinyl, N-(N, N-dimethyl glycinyl)-leucinyl, N-(8-quinoline sulfonyl)-leucinyl, N-Cbz-leucinyl, N-pentafluorobenzoyl-leucinyl, N-2-naphthoyl-leucinyl, N-1-naphthoyl-leucinyl, N-4-fluorobenzoyl-leucinyl, N-(4-trifluoromethyl benzoyl)-leucinyl N-3,4-difluorobenzoyl-leucinyl, N-3,4-dimethoxybenzoyl-leucinyl, N-(1-benzothiophene-carbonyl)-leucinyl, N-(2-benzothiazole-carbonyl)-leucinyl, N-(5-benzothiophene-carbonyl)-leucinyl, N-(6-benzothiophen e-carbonyl)-leucinyl, N-(5-indole-carbonyl)-leucinyl, N-(trans-4-propyl cyclohexyl-carbony)-leucinyl, N-(2-quinoxaline-carbonyl)-leucinyl, N-5-(2,3-dihydro-benzofuran) carbonyl)-leucinyl, N-(2-benzofuran-carbonyl)-leucinyl, N-(N-methyl-2-indole-carbonyl)-leucinyl, N-(2-chloro-benzoyl-carbonyl)-leucinyl, N-(4-phenoxy-phenyl-carbonyl)-leucinyl, N-(3-methoxy-2-quinoline-carbonyl)-leucinyl, N-(2-pyridyl-methyleneoxy-caboonylyleucinyl or N-(cyclohexyl-carbonyl)-leucinyl, N-Cbz-norleucinyl, N-(2-naphthyl-carbonyl)-norleucinyl, N-(3,4-dimethoxy-benzoyl)-norleucinyl, N-(5-benzothiophene-carbonyl)-norleucinyl, N-Cbz-norvalinyl, N-Cbz-isoleucinyl, N-Cbz-α-allyl-glycinyl, N-Cbz-N-methyl-leucinyl-, N-Cbz-α-(cyclopropylmethyl)-glycinyl-, 2-(3-biphenyl)-4methyl-valeryl, 1-(3-biphenyl)-but-3-ene-1-carbonyl, or 1-(3-biphenyl)-ethyl-2-cyclopropane-1-carbonyl;
R⁵ is selected from the group consisting of N-Cbz-norvalinyl-, 3-(2-pyridyl)-phenyl acetyl, 3-(3-pyridyl)-phenyl acetyl, 1-(3-biphenyl)-3-methyl-but-3-ene-1-carbonyl, 1-(3-biphenyl)-but-3-ene-1-carbonyl, 2-pyridyl sulfonyl, 8-quinoline sulfonyl-, 1,3-dimethyl-5-chloro-pyrazole-4-sulfonyl, 3,5-dimethyl-isoxazolesulfonyl, benzo-2,1,3-thiadiazole-4- sulfonyl, 3-biphenyl sulfonyl, 8-quinolone carbonyl, 5-(2-pyridine)-thiophene-carbonyl, N-benzyl-4-piperidinyl carbonyl, 2-quinoline carbonyl, 2-pyridine-carbonyl, 4-phenoxy-phenyl-carbonyl, 2-(3-biphenyl)-3-methyl-valeryl, 2-carboxymethyl-phenyl-sulfonyl, 2-carboxyl-phenyl-sulfonyl, 4-C-tetrazole-phenyl-sulfonyl, 1-naphthalene-sulfonyl, 2-cyano-phenyl-sulfonyl, or phenyl.

12. A compound according to claim 1 wherein:
R¹ is H or C₁₋₆ alkyl;
R² and R³ are H;
R⁴ is N—(R⁶)—NHCH(C₁₋₆ alkyl)-CO or Ar—C₁₋₆ alkyl-CO; and
R⁵ is Ar—C₁₋₆ alkyl-CO or Het-SO₂.

13. A compound according to claim 12 wherein R⁴ is N—(R⁶)—NHCH(C₁₋₆ alkyl)-CO is N—R⁶-leucinyl or N—R⁶-norleucinyl.

14. A compound according to claim 12 wherein:
R¹ is H or Me;
R⁴ is selected from the group consisting of Cbz-leucinyl, 2-naphthoyl-leucinyl, 4-fluorobenzoyl-leucinyl, 3,4-dimethoxybenzoyl-leucinyl, (1-benzothiophene-carbonyl)-leucinyl, (2-quinoxaline-carbonyl)-leucinyl, 5-(2,3-dihydro-benzofuran)-carbonyl)-leucinyl, (2-benzofuran-carbonyl)-leucinyl, (2-naphthyl-carbonyl)- norleucinyl, (3,4-dimethoxy-benzoyl)-norleucinyl, (5-benzothiophene-carbonyl)-norleucinyl, and 2-(3-biphenyl)-4methyl-valeryl; and
R⁵ is 3-(2-pyridyl)-phenyl acetyl or 2-pyridyl sulfonyl.

15. A compound of claim 1 selected from the group consisting of:
1-N-(N-(2-pyridyl carbonyl)-leucinyl)-amino-3-N-(2-pyridyl-sulfonyl)-amino-propan-2-one;
1-N-(N-(8-quinoline carbonyl)-leucinyl)-amino-3-N-(2-pyridyl-sulfonyl)-amino-propan-2-one;
1-N-(N-(2-quinoline carbonyl)-leucinyl)-amino-3-N-(2-pyridyl-sulfonyl)-amino-propan-2-one;
1-N-(N-(4imidazole acetyl)-leucinyl)-amino-3-N-(3-biphenyl sulfonyl)-amino-propan-2-one;
1-N-(N-(2-pyridyl-carbonyl)-leucinyl)-amino-3-N-(8-quinoline carbonyl)-amino-propan-2-one;
1-N-(N-benzoyl-leucinyl)-amino-3-N-(8-quinoline carbonyl)-amino-propan-2-one;
1-N-(N-(2-pyridyl sulfonyl)-leucinyl)-amino-3-N-(8-quinoline carbonyl)-amino-propan-2-one;
1-N-(N-(8-quinoline carbonyl)-leucinyl)-amino-3-N-(8-quinoline carbonyl)-amino-propan-2-one;
1-N-(N-(1-isoquinoline-carbonyl)-leucinyl)-amino-3-N-(8-quinoline carbonyl)-amino-propan-2-one;
1-N-(N-(N-morpholine-acetyl)-leucinyl)-amino-3-N-(8-quinoline carbonyl)-amino-propan-2-one;
1-N-(N-(N-metbyl prolinyl)-leucinyl)-amino-3-N-(8-quinoline carbonyl)-amino-propan-2-one;
1-N-(N-(N, N-dimethyl glycinyl)-leucinyl)-amino-3-N-(8quinoline carbonyl)-amino-propan-2-one;
1-N-(N-(8-quinoline sulfonyl)-leucinyl)-amino-3-N-(8-quinotine carbonyl)-amino-propan-2-one;
1-N-(N-Cbz-leucinyl)-amino-3-N-(3-(2-pyridyl)-phenyl acetyl)-amino-propan-2-one;
1-N-(N-pentafluorobenzoyl-leucinyl)-amino-3-N-(3-(2-pyridyl)-phenyl acetyl)-amino-propan-2-one;
1-N-(N-2-naphthoyl-leucinyl)-amino-3-N-(3-(2-pyridyl)-phenyl acetyl)-amino-propan-2-one;
1-N-(N-1-naphthoyl-leucinyl)-amino-3-N-(3-(2-pyridyl)-phenyl acetyl)-amino-propan-2-one;
1-N-(N-(2-pyridyl-carbonyl)-leucinyl)-amino-3-N-(3-(2-pyridyl)-phenyl acetyl)-amino-propan-2-one;
1-N-(N-4-fluorobenzoyl-leucinyl)-amino-3-N-(3-(2-pyridyl)-phenyl acetyl)-arino-propan-2-one;
1-N-(N-3,4-difluorobenzoyl-leucinyl)-amino-3-N-(3-(2-pyridyl)-phenyl acetyl)-amino-propan-2-one;
1-N-(N-3,4-dimethoxybenzoyl-leucinyl)-amino-3-N-(3-(2-pyridyl)-phenyl acetyl)-amino-propan-2-one;
1-N-(N-(1-benzothiophene-carbonyl)-leucinyl)-amino-3-N-(3-(2-pyridyl)-phenyl acetyl)-amino-propan-2-one;
1-N-(N-(5-indole-carbonyl)-leucinyl)-amino-3-N-(3-(2-pyridyl)-phenyl acetyl)-amino-propan-2-one;
1-N-(N-Cbz-isoleucinyl)-amino-3-N-(3-(2-pyridyl)-phenyl acetyl)-amino-propan-2-one;
1-N-(N-Cbz-norvalinyl)-amino-3-N-(3-(2-pyridyl)-phenyl acetyl)-amino-propan-2-one;
1-N-(N-Cbz-α-allyl-glycinyl)-amino-3-N-(3-(2-pyridyl)-phenyl acetyl)-amino-propan-2-one;
1-N-(N-Cbz-norleucinyl)-amino-3-N-(3-(2-pyridyl)-phenyl acetyl)-amino-propan-2-one;
1-N-(N-Cbz-N-methyl-leucinyl)-amino-3-N-(3-(2-pyridyl)-phenyl acetyl)-amino-propan-2-one;
1-N-(N-Cbz-α-(cyclopropyl)-methyl-glycinyl)-amino-3-N-(3-(2-pyridyl)-phenyl acetyl)-amino-propan-2-one;
1-N-(N-benzyloxycarbonyl-L-β-tert-butylalanine)-amino-3-N-(3-(2-pyridyl)-phenyl acetyl)-amino-propan-2-one;
1-N-(2-(3-biphenyl)-4-methyl-valeryl)-amino-3-N-(2-pyridyl-sulfonyl)-amino-propan-2-one;
1-N-(2-(3-biphenyl)-4-methyl-valeryl)-amino-3-N-(2-carboxymethyl-phenyl-sulfonyl)-amino-propan-2-one;
1-N-(2-(3-biphenyl)-4-methyl-valeryl)-amino-3-N-(4-cyano-phenyl-sulfonyl)-amino-propan-2-one;
1-N-(2-(3-biphenyl)-4-methyl-valeryl)-amino-3-N-(8-quinoline carbonyl)-amino-propan-2-one;
1-N-(2-(3-biphenyl)-4-methyl-valeryl)-amino-3-N-(3-(2-pyridyl)-phenyl acetyl)-amino-propan-2-one;
1-N-(2-(3-biphenyl)-4-methyl-valeryl)-amino-3-N-(3-(3-pyridyl)-3-phenyl acetyl)-amino-propan-2-one;
1-N-(2-(3-biphenyl)-4-methyl-valeryl)-amino-3-N-(2-pyridine carbonyl)-amino-propan-2-one;
1-N-(2-(3-biphenyl)-4-methyl-valeryl)-amino-3-N-(5-(2-pyridine)-thiophene-carbonyl)-amino-propan-2-one;
1-N-(2-(3-biphenyl)-4methyl-valeryl)-amino-3-N-(N-benzyl-4-piperidine-carbonyl)-amino-propan-2-one;
1-N-(2-(3-biphenyl)-4-methyl-valeryl)-amino-3-N-(2-quinoline-carbonyl)-amino-propan-2-one;
1-N-(2-(3-biphenyl)-4-methyl-valeryl)-amino-3-N-(2-carboxyl-phenyl-sulfonyl)-amino-propan-2-one;
1-N-(2-(3-biphenyl)-methyl-valeryl)-amino-3-N-(4-C-tetrazole-phenyl-sulfonyl)-amino-propan-2-one;
1-N-(2-(3-biphenyl)-4methyl-valeryl)-amino-3-N-(3-(2-pyridyl-(phenyl acetyl)-amino-(S)-butan-2-one;
1-N-(2-(3-biphenyl)-3-methyl-valeryl)-1-N-methyl-amino-3-N-(3-(2-pyridyl-(phenyl acetyl)-amino-propan-2-one;
1-N-(N-2-pyridyl carbonyl-leucinyl)-amino-3-N-(4-phenoxy-phenyl carbonyl)-amino-propan-2-one;
1-N-(N-8-quinoline-carbonyl-leucinyl)-amino-3-N-(4phenoxy-phenyl carbonyl)-amino-propan-2-one;
1-N-(N-2-quinoline-carbonyl-leucinyl)-amino-3-N-(4-phenoxy-phenyl carbonyl)-amino-propan-2-one;
1-N-(N-(Cbz-norvalinyl)-amino-3-N-(8-quinoline-sulfonyl)-amino-propan-2-one;
1-N-(8-quinoline-sulfonyl)-amino-3-N-(8-quinoline-sulfonyl)-ano-propan-2-one;
1-N-(2-(3-biphenyl)-3-methyl-valeryl)-amino-3-N-(8-quinoline-sulfonyl)-amino-propan-2-one;
1-N-(2-(3-biphenyl)-3-methyl-valeryl)-amino-3-N-(2-(3-biphenyl)-3-methyl-valeryl) amino-propan-2-one;
1-N-(N-(Cbz-norvalinyl)-amino-3-N-(N-(Cbz-norvalinyl)-amino-propan-2-one;
1-N-(1-(3-biphenyl)-but-3-ene-1-carbonyl)-amino-3-N-(3-(2-pyridyl)-phenyl acetyl)-amino-propan-2-one;
1-N-(1-(3-biphenyl)-but-3-ene-1-carbonyl)-amino-3-N-(1-(3-biphenyl)-but-3-ene-1-carbonyl)-propan-2-one;
1-N-(3-biphenyl)-ethyl-2-cyclopropane-1-carbonyl)-amino-3-N-(3-(2-pyridyl)-phenyl acetyl)-amino-propan-2-one;
1-N-(1-(3-biphenyl)-3-methyl-but-3-ene-1-carbonyl)-amino-3-N-(3-(2-pyridyl)-phenyl acetyl)-amino propan-2-one;
1-N-(1-(3-biphenyl)-3-methyl-but-3-ene-1-carbonyl)-amino)-3-N-(1-(3-biphenyl)-3-methyl-but-3-ene-1-carbonyl)-amino-propan-2-one;
1-N-(N-(trans-4propyl cyclohexyl-carbonyl)-leucinyl)-amino-3-N-(3-(2-pyridyl)-phenyl acetyl)-amino-propan-2-one;
1-N-(N-(2-quinoxaline-carbonyl)-leucinyl)-amino-3-N-(3-(2-pyridyl)-phenyl acetyl)-amino-propan-2-one;
1-N-(N-(5-(2,3-dihydro-benzofuran)-carbonyl)-leucinyl)-amino-3-N-(3-(2-pyridyl)-phenyl acetyl)-amino-propan-2-one;
1-N-(N-(N-methyl-2-indole-carbonyl)-leucinyl)-amino-3-N-(3-(2-pyridyl)-phenyl acetyl)-amino-propan-2-one;
1-N-(N-(cyclohexyl-carbonyl)-leucinyl)-amino-3-N-(3-(2-pyridyl)-phenyl acetyl)-amino-propan-2-one;
1-N-(N-(2-chloro-benzoyl)-leucinyl)-amino-3-N-(3-(2-pyridyl)-phenyl acetyl)-amino-propan-2-one;
1-N-(N-(2-benzofuran-carbonyl)-leucinyl)-amino-3-N-(3-(2-pyridyl)-phenyl acetyl)-amino-propan-2-one;
1-N-(N-(3-phenoxy-phenyl-carbonyl)-leucinyl)-amino-3-N-(3-(2-pyridyl)-phenyl acetyl)-amino-propan-2-one;
1-N-(N-(4-phenoxy-phenyl-carbonyl)-leucinyl)-amino-3-N-(3-(2-pyridyl)-phenyl acetyl)-amino-propan-2-one;
1-N-(N-(3-methoxy-2-quinoline-carbonyl)-leucinyl)-amino-3-N-(3-(2-pyridyl)-phenyl acetyl)-amino-propan-2-one;
1-N-(N-Cbz-leucinyl)-amino-3-N-(3-(2-pyridyl-(phenyl acetyl)-amino-(S)-butan-2-one;
1-N-(N-(4-fluorobenzoyl)-leucinyl)-amino-3-N-(3-(2-pyridyl-(phenyl acetyl)-amino-(S)-butan-2-one;
1-N-(N-(2-benzothiophene-carbonyl)-leucinyl)-amino-3-N-(3-(2-pyridyl-(phenyl acetyl)-amino-(S)-butan-2-one;
1-N-(N-(2-pyridyl-methyleneoxy-carbonyl)-leucinyl)-amino-3-N-(1-napbthalene sulfonyl)-amino-propan-2-one;
1-N-(N-(2-pyridyl-methyleneoxy-carbonyl)-leucinyl)-amino-3-N-(1,3-dimethyl-5-chloro-pyrazole-4sulfonyl)-amino-propan-2-one;
1-N-(N-(2-pyridyl-methyleneoxy-carbonyl)-leucinyl)-amino-3-N-(benzo-2,1,3-thiadiazole-4-sulfonyl)-amino-propan-2-one;
1-N-(N-(2-pyridyl-methyleneoxy-carbonyl)-leucinyl)-amino-3-N-(3,5-dimethyl-isoxazole-4-sulfonyl)-aniino-propan-2-one;
1-N-(N-(4-trifluoromethyl benzoyl)-leucinyl)-amino-3-N-(3-(2-pyridyl)-phenyl acetyl)-amino-propan-2-one;
1-N-(N-(6-benzthiazole-carbonyl)-leucinyl)-amino-3-N-(3-(2-pyridyl)-phenyl acetyl)-amino-propan-2-one;
1-N-(N-(6-quinoline-carbonyl)-leucinyl)-amino-3-N-(3-(2-pyridyl)-phenyl acetyl)-amino-propan-2-one;
1-N-(N-(4-fluoro-benzoyl)-norleucinyl)-amino-3-N-(3-(2-pyridyl)-phenyl acetyl)-amino-propan-2-one;
1-N-(N-(2-naphthyl-carbonyl)-norleucinyl)-amino-3-N-(3-(2-pyridyl)-phenyl acetyl)-amino-propan-2-one;
1-N-(N-(3,4-dimethoxy-benzoyl)-norleucinyl)-amino-3-N-(3-(2-pyridyl)-phenyl acetyl)-amino-propan-2-one;
1-N-(N-(5-benzothiophene-carbonyl)-norleucinyl)-amino-3-N-(3-(2-pyridyl)-phenyl acetyl)-amino-propan-2-one; AND
(S)-3-N-(N-Cbz-leucinyl)-amino-1-N-(phenyl)-5-methyl-hexan-2-one.

16. A compound of claim 15 selected from the group consisting of:
1-N-(N-Cbz-leucinyl)-amino-3-N-(3-(2-pyridyl)-phenyl acetyl)-amino-propan-2-one;
1-N-(N-2-naphthoyl-leucinyl)-amino-3-N-(3-(2-pyridyl)-phenyl acetyl)-amino-propan-2-one;
1-N-(N-4-fluorobenzoyl-leucinyl)-amino-3-N-(3-(2-pyridyl)-phenyl acetyl)-amino-propan-2-one;
1-N-(N-3,4dimethoxybenzoyl-leucinyl)-amino-3-N-(3-(2-pyridyl)-phenyl acetyl)-amino-propan-2-one
1-N-(N-(1-benzothiophene-carbonyl)-leucinyl)-amino-3-N-(3-(2-pyridyl)-phenyl acetyl)-amino-propan-2-one;
1-N-(N-(5-indole-carbonyl)-leucinyl)-amino-3-N-(3-(2-pyridyl)-phenyl acetyl)-amino-propan-2-one;
1-N-(2-(3-biphenyl)-4methyl-valeryl)-amino-3-N-(2-pyridyl-sulfonyl)-amino-propan-2-one;
1-N-(2-(3-biphenyl)-4methyl-valeryl)-amino-3-N-(3-(2-pyridyl)-phenyl acetyl)-amino-propan-2-one;
1-N-(N-(2-quinoxaline-carbonyl)-leucinyl)-amino-3-N-(3-(2-pyridyl)-phenyl acetyl)-amino-propan-2-one;
1-N-(N-(5-(2,3-dihydro-benzofuran)-carbonyl)-leucinyl)-amino-3-N-(3-(2-pyridyl)-phenyl acetyl)-amino-propan-2-one;
1-N-(N-(2-benzofuran-carbonyl)-leucinyl)-amino-3-N-(3-(2-pyridyl)-phenyl acetyl)-amino-propan-2-one;
1-N-(N-Cbz-leucinyl)-amino-3-N-(3-(2-pyridyl-(phenyl acetyl)-amino-(S)-butan-2-one;
1-N-(N-(2-benzothiophene-carbonyl)-leucinyl)-amino-3-N-(3-(2-pyridyl-(phenyl acetyl)-amino-(S)-butan-2-one;
1-N-(N-(4-trifluoromethyl benzoyl)-leucinyl)-amino-3-N-(3-(2-pyridyl)-phenyl acetyl)-amino-propan-2-one;
1-N-(N-(2-naphthyl-carbonyl)-norleucinyl)-amino-3-N-(3-(2-pyridyl)-phenyl acetyl)-amino-propan-2-one;
1-N-(N-(3,4-dimethoxy-benzoyl)-norleucinyl)-amino-3-N-(3-(2-pyridyl)-phenyl acetyl)-amino-propan-2-one; and
1-N-(N-(5-benzothiophene-carbonyl)-norleucinyl)-amino-3-N-(3-(2-pyridyl)-phenyl acetyl)-amino-propan-2-one.

17. A pharmaceutical composition comprising a compound according to claim 1 and a pharmaceutically acceptable carrier, diluent or excipient.

18. A pharmaceutical composition comprising a compound according to claim 16 and a pharmaceutically acceptable carrier, diluent or excipient.

19. A method of inhibiting a protease selected from the group consisting of a cysteine protease and a serine protease, comprising administering to a patient in need thereof an effective amount of a compound according to claim 1.

20. A method of inhibiting a protease selected from the group consisting of a cysteine protease and a serine protease, comprising administering to a patient in need thereof an effective amount of a compound according to claim 16.

21. A method according to claim 19 wherein said protease is a cysteine protease.

22. A method according to claim 20 wherein said protease is a cysteine protease.

23. A method according to claim 21 wherein said cysteine protease is cathepsin K.

24. A method according to claim 22 wherein said cysteine protease is cathepsin K.

25. A method of treating a disease characterized by bone loss comprising inhibiting said bone loss by administering to a patient in need thereof an effective amount of a compound according to claim 1.

26. A method according to claim 25 wherein said disease is osteoporosis.

27. A method according to claim 25 wherein said disease is periodontitis.

28. A method according to claim 25 wherein said disease is gingivitis.

29. A method of treating a disease characterized by excessive cartilage or matrix degradation comprising inhibiting said excessive cartilage or matrix degradation by administering to a patient in need thereof an effective amount of a compound according to claim 1.

30. A method according to claim 29 wherein said disease is osteoarthritis.

31. A method according to claim 29 wherein said disease is rheumatoid arthritis.

32. A method of treating a disease characterized by bone loss comprising inhibiting said bone loss by administering to a patient in need thereof an effective amount of a compound according to claim 16.

33. A method according to claim 32 wherein said disease is osteoporosis.

34. A method according to claim 32 wherein said disease is periodontitis.

35. A method according to claim 32 wherein said disease is gingivitis.

36. A method of treating a disease characterized by excessive cartilage or matrix degradation comprising inihibiting said excessive cartilage or matrix degradation by administering to a patient in need thereof an effective amount of a compound according to claim 16.

37. A method according to claim 36 wherein said disease is osteoarthritis.

38. A method according to claim 36 wherein said disease i s rheumatoid art hritis.

39. A compound of Fornula II: 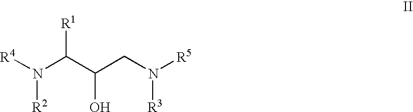
wherein:
R¹, R² and R³ are independently selected from the group consisting of H, C₁₋₆ alkyl, C₃₋₁₁cycloalky, C₂₋₆ alkenyl, C₂₋₆ alkyny, Ar; Het, C₁₋₆ alkyl-Ar, C₃₋₁₁cycloalkyl-Ar, C₂₋₆ alkenyl-Ar, C₂₋₆ alkynyl-Ar; C₁₋₆ alkyl-Het, C₃₋₁₁cycloalkyl-Het, C₂₋₆ alkenyl-Het, and C₂₋₆ alkynyl-Het;
R⁴ is selected from the group consisting of N—(R⁶(—NHCH(C₁₋₆ alkyl)-CO, N,N—R⁶—(C₁₋₆ alkyl)—N(C₁₋₆ alkyl)-CO, N—(R⁶)—NHCH(C₂₋₆ alkenyl)-CO—, N—(R⁶)—NHCH(C₂₋₆alkynyl)-CO—, N—(R⁶)—NHCH(C₁₋₆ alkyl-Ar)—CO—, N—(R⁶)—NHCH(C₂₋₆ alkenylAr)—CO—, N—(R⁶)—NHCH(C₂₋₆ alkynyl-Ar)—CO—, N—(R⁶)—NHCH(C₁₋₆ alkyl-Het)-CO—, N—(R⁶)—NHCH(C₂₋₆ alkenyl-Het)-CO—, N—(R⁶)—NHCH(C₂₋₆ alkynyl-Het)-CO—, ArCO, Ar—C₁₋₆ alkyl-CO, Ar—SO₂, Ar—C₁₋₆ alkyl-SO₂, Het-CO, Het-C₁₋₆ alkyl-CO, Het-SO₂, and Het-C₁₋₆alkyl-SO₂;
R⁵ is selected from the group consisting of N—R⁷-amino acid, C₁₋₆ alkyl CO, C₃₋₁₁cycloalkyl-CO, ArCO, Ar—C₁₋₆ alkyl-CO, Ar—SO₂, Ar—C₁₋₆ alkyl-SO₂, Het-CO, Het-C₁₋₆ alkyl-CO, Het-SO₂, C₁₋₆ alkyl, Ar—C₀₋₆ alkyl-, and Het-C₀₋₆ alkyl-.
R⁶ and R⁷ are independently selected from the group consisting of Ar—(C₁₋₆ alkyl)-O—CO, Het-(C₁₋₆ alkyl)-O—CO, Ar—CO, Ar—SO₂, Het-CO, Het-SO₂, C₁₋₆ alkyl-CO, C₃₋₁₁cycloalkyl-CO, C₁₋₆ alkyl-SO₂, C₂₋₆ alkenyl-CO,C₂₋₆ alkenyl-SO₂, C₂₋₆ alkynyl-CO; C₂₋₆ alkynyl-SO₂, ArC₁₋₆ alkyl-CO, ArC₁₋₆ alkyl-SO₂, ArC₂₋₆ alkenyl-CO, ATC₂₋₆ alkenyl-SO₂, Ar—C₂₋₆ alkyl-CO,Ar—C₂₋₆ alkynyl-SO₂, Het-C₁₋₆ alkyl-CO, Het-C₁₋₆ alkyl-SO₂, Het-C₂₋₆ alkenyl-CO, Het-C₂₋₆ alkenyl-SO₂, Het-C₂₋₆ alkynyl-CO, and Het-C₂₋₆ alkynyl-SO₂;
and pharmaceutically acceptable salts, hydrates and solvates thereof.

40. A compound according to claim 39 wherein R¹, R² and R³ are independently selected from the group consisting of methyl isobutyl, phenyl, benzyl, and isonicotinyl.

41. A compound according to claim 39 wherein R¹, R² and R³ are H.

42. A compound according to claim 39 wherein R⁴ is selected from the group consisting of N—R⁶-leucinyl, N—R⁶-norleucinyl-, N—R⁶-norvalinyl-, N—R⁶-isoleucinyl-, N—R⁶-at-allyl-glycinyl-, N—R⁶-α-(cyclopropylmethyl)-glycinyl-, N—R⁶-β-tert-butyl-alaninyl-2-, N—R⁶-homo-leucinyl-, N,N—R⁶-methyl-leucinyl-, 3-phenoxy-benzoyl, 4-phenoxy-benzoyl-, or 2-benzyloxy-benzoyl, 4-biphenyl acetyl-, 2-(4-biphenyl)-4-methyl-valeryl, 2-(3-biphenyl)-4-methyl-valeryl, 1-(3-biphenyl)-but-3-ene-1-carbonyl, 1-(3-biphenyl)-ethyl-cyclopropane-1-carbonyl, 1-(3-biphenyl)-3-methyl-but-3-ene-1-carbonyl, 3-(2-pyridyl)-phenyl acetyl, 3-(3-pyridyl)-phenyl acetyl, 3-phenoxy-phenyl sulfonyl, 4-phenoxy-phenyl sulfonyl, 3-(4-(3-chloro-2-cyano-phenoxy)-phenyl sulfonyl, and 8-quinoline sulfonyl-.

43. A compound according to claim 39 wherein N—R⁷-amino acid is selected from the group consisting of N—(R⁷)—NHCH(C₁₋₆ alkyl)-CO, N—(R⁷)—NHCH(C₂₋₆ alkenyl)-CO—, N—(R⁷)—NHCH(C₂₋₆ alkynyl)-CO—, N—(R⁷)—NHCH(C₁₋₆ alkyl-Ar)—CO—, N—(R⁷)—NHCH(C₂₋₆ alkenylAr)—CO—, N—(R⁷)—NHCH(C₂₋₆ alkynyl-Ar)—CO—, R⁷-γ-t-butyl-glutamyl-, R⁷-glutarnyl-, and N,N—R⁷—(C₁-C₆ alkyl)-leucinyl-.

44. A compound according to claim 39 wherein R⁵ is selected from the group consisting of N—R⁷-leucinyl-, N—R⁷-norleucinyl-, N—R⁷-norvalinyl-,N—R⁷-isoleucinyl-, N—R⁷-α-allyl-glycinyl-, N—R⁷-α-(cyclopropylmethyl)-glycinyl-, N—R⁷—O-tert-butyl-alaninyl-, N—R⁷-homo-leucinyl, N—(R⁷)-phenylalaninyl, acetyl, benzoyl, 3-phenoxy-benzoyl, 4-phenoxy-benzoyl, 2-benzyloxy benzoyl, 3-benzyloxy benzoyl, or 4-benzyloxy benzoyl, 2-(4-biphenyl)-4methyl-valeryl, 2-(3-biphenyl)-4methyl-valeryl, 1-(3-biphenyl)-but-3-ene-1-carbonyl, 1-(3-biphenyl)-ethyl-cyclopropane-1-carbonyl, 1-(3-biphenyl)-3-methyl-but-3-ene-1-carbonyl, 3-(2-pyridyl)-phenyl acetyl, 3-(3-pyridyl)-phenyl acetyl, 4-biphenyl acetyl-, 3-biphenyl acetyl-, 8-quinoline sulfonyl-, 3-biphenyl sulfonyl-, 4-cyano-phenyl sulfonyl, 2-carboxyl-phenyl sulfonyl, 2-carboxymethyl-phenyl sulfonyl-, 4-C-tetrazole-phenyl sulfonyl, 1-naphthalene sulfonyl, 3-phenoxy-phenyl sulfonyl, 4-phenoxy-phenyl sulfonyl, 3-(4-(3-chloro-2-cyano-phenoxy)-phenyl sulfonyl-, 4biphenyl sulfonyl-, 2dibenzofuran-sulfonyl, 8-quinoline carbonyl-, 6-quinoline carbonyl-, 2-pyridine carbonyl, 5-(2-pyridyl)-thiophene carbonyl, N-benzylpiperidinyl carbonyl, 2-quinoline carbonyl-, 2-pyridyl sulfonyl, 1,3-dimethyl-5-chloro-pyrazole-4-sulfonyl, 3,5-dimethyl-isoxazole-4-sulfonyl, benzo-2,1,3-thiadiazoleX sulfonyl, phenyl-sulfone-5-thiophene-2-sulfonyl-, 2-carboxymethyl thiophene-sulfonyl, 2,5-dichlorothiophene-3-sulfonyl-, and phenyl.

45. A compound according to claim 39 wherein R⁶ and R⁷ are independently selected from the group consisting of benzyloxycarbonyl, 2-pyridyl methyloxycarbonyl, 3-pyridyl methyloxycarbonyl, 4pyridyl methyloxycarbonyl, benzoyl-, 1-naphthoyl-, 2-naphthoyl-, 4-phenoxy-benzoyl-, 3-phenoxy-benzoyl-, 2-phenoxy-benzoyl-, 2-chloro-benzoyl-, 4-fluoro-benzoyl, 3,4-difluoro benzoyl-, 4trifluoromethyl benzoyl-, 2-chlorobenzoyl-, 4-carboxymethyl-benzoyl-, 4-carboxyl-benzoyl-, N,N-dimethyl glycinyl-, 2-pyridyl carbonyl-, 3-pyridyl carbonyl, 4-pyridyl carbonyl-, 2-quinoline carbonyl-, 3-quinoline carbonyl-, 4-quinoline carbonyl-, 5-quinoline carbonyl-, 6-quinoline carbonyl-, 7-quinoline carbonyl-, 8-quinoline carbonyl-, 1-isoquinoline carbonyl-, 3- isoquinone carbonyl-, 4 isoquinohne carbonyl-, 5- isoquinoline carbonyl-, 6- isoquinoline carbonyl-, 7- isoquinoline carbonyl-, 8-isoquinoline carbonyl-, 1-benzothiophene carbonyl-, 1-benzofurancarbonyl-, 5-indole-carbonyl-sulfonyl-, N-methyl-prolinyl-, 2-quinoxaline-carbonyl-, 5-(2,3-dihydrobenzofurancarbonyl-, 2-benzofuran-carbonyl-, 2-benzothiophene-carbonyl-, N-morpholino-carbonyl-, N-methyl-piperidine-carbonyl-, N-pyrazole-carbonyl-, 2-pyridyl sulfonyl-, 3-pyridyl sulfonyl, 4-pyridyl sulfonyl, 2-quinoline sulfonyl-, 3-quinoline sulfonyl-, 4quinoline sulfonyl-, 5-quinoline sulfonyl-, 6-quinoline sulfonyl-, 7-quinoline sulfonyl-, 8-quinoline sulfonyl-, 1- isoquinoline sulfonyl-, 3- isoquinoline sulfonyl-, 4- isoquinoline sulfonyl-, 5-isoquinoline sulfonyl-, 6- isoquinoline sulfonyl-, 7- isoquinoline sulfonyl-, 8- isoquinoline sulfonyl-, acetyl, trans-4-propyl-cyclohexyl-carbonyl-, cyclohexyl-carbonyl-, 4-imidazole acetyl-, 2-pyridyl acetyl, 3-pyridyl acetyl, 4-pyridyl acetyl-, and N-morpholine acetyl.

46. Use of a compound according to any one of claims 1 to 16 in the manufacture of a medicament for use in inhibiting a protease selected from the group consisting of a cysteine protease and a serine protease.

47. A use according to claim 46 wherein said protease is a cysteine protease.

48. A use according to claim 47 wherein said cysteine protease is cathepsin K.

49. Use of a compound according to any one of claims 1 to 16 in the manufacture of a medicament for use in treating a disease characterized by bone loss.

50. A use according to claim 49 wherein said disease is osteoporosis.

51. A use according to claim 49 wherein said disease is periodontitis.

52. A use according to claim 49 wherein said disease is gingivitis.

53. Use of a compound according to any one of claims 1 to 16 in the manufacture of a medicament for use in treating a disease characterized by excessive cartilage or matrix degradation.

54. A use according to claim 53 wherein said disease is osteoarthritis.

55. A use according to claim 53 wherein said disease is rheumatoid arthritis.
